(19) United States
(12) Patent Application Publication
(10) Pub. No.: US 2012/0269821 A1
(43) Pub. Date: Oct. 25, 2012

(54) HIV-1 ANTIBODIES

(76) Inventors: Barton F. Haynes, Durham, NC (US); Hua-Xin Liao, Durham, NC (US); M. Anthony Moody, Durham, NC (US); Lynn Morris, Sandringham (ZA); Salim S. Abdool Karim, Glenwood (ZA)

(21) Appl. No.: 13/314,712

(22) Filed: Dec. 8, 2011

Related U.S. Application Data
(63) Continuation of application No. PCT/US2010/002770, filed on Oct. 18, 2010.
(60) Provisional application No. 61/272,654, filed on Oct. 16, 2009.

(51) Int. Cl. C07K 16/10; C12N 15/13; C12P 21/02; C12N 15/63; C12N 5/10; A61K 39/42; A61P 31/18

(52) U.S. Cl. 424/160.1; 530/389.4; 530/388.35; 530/387.3; 536/23.53; 435/320.1; 435/326; 435/69.6

(57) ABSTRACT

The present invention relates, in general, to HIV-1 antibodies and, in particular, to broadly neutralizing HIV-1 antibodies that target the gp41 membrane-proximal external region (MPER).

This application is a continuation of International Application No. PCT/US/2010/002770, filed Oct. 18, 2010, which claims priority from U.S. Prov. Application No. 61/272,654, filed Oct. 16, 2009, the entire contents of which are incorporated herein by reference.

This invention was made with government support under Grant No. AI 0678501, awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 13, 2012, is named 15791773.txt and is 63,494 bytes in size.

### TECHNICAL FIELD

The present invention relates, in general, to HIV-1 specific antibodies and, in particular, to broadly neutralizing HIV-1 specific antibodies that target the gp41 membrane-proximal external region (MPER).

### BACKGROUND

The development of strategies to utilize human antibodies that potently inhibit HIV-1 infection of T cells and mononuclear phagocytes is a high priority for treatment and prevention of HIV-1 infection (Mascola et al, J. Virol. 79:10103-10107 (2005)). A few rare human monoclonal antibodies (mAbs) against gp160 have been isolated that can broadly neutralize HIV-1 in vitro, and can protect non-human primates from SHIV infections in vivo (Mascola et al, Nat. Med. 6:207-210 (2000), Baba et al, Nat. Med. 6:200-206 (2000)). These mAbs include antibodies 2F5 and 4E10 against the membrane proximal external region (MPER) of gp41 (Muster et al, J. Viral. 67:6642-6647 (1993), Stiegler et al, AIDS Res. & Hum. Retro. 17:1757-1765 (2001), Zwick et al, J. Virol. 75:10892-10905 (2001)), IgG1b12 against the CD4 binding site of gp120 (Roben et al, J. Virol. 68:4821-4828 (1994)), and mAb 2G12 against gp120 high mannose residues (Sanders et al, J. Virol. 76:7293-7305 (2002)).

HIV-1 has evolved a number of effective strategies for evasion from neutralizing antibodies, including glycan shielding of neutralizing epitopes (Wei et al, Nature 422:307-312 (2003)), entropic barriers to neutralizing antibody binding (Kwong et al, Nature 420:678-682 (2002)), and masking or diversion of antibody responses by non-neutralizing antibodies (Alam et al, J. Virol. 82:115-125 (2008)). Despite intense investigation, it remains a conundrum why broadly neutralizing antibodies against either the gp120 CD4 binding site or the membrane proximal region of gp41 are not routinely induced in either animals or man.

One clue as to why broadly neutralizing antibodies are difficult to induce may be found in the fact that all of the above-referenced mAbs have unusual properties. The mAb 2G12 is against carbohydrates that are synthesized and modified by host glycosyltransferases and are, therefore, likely recognized as self carbohydrates (Calarese et al, Proc. Natl. Acad. Sci. USA 102:13372-13377 (2005)). 2G12 is also a unique antibody with Fabs that assemble into an interlocked VH domain-swapped dimers (Calarese et al, Science 300:2065-2071 (2003)). 2F5 and 4E10 both have long CDR3 loops, and react with multiple host antigens including host lipids (Zwick et al, J. Virol. 75:10892-10905 (2001), Alam et al, J. Immun. 178:4424-4435 (2007), Zwick et al, J. Virol. 78:3155-3161 (2004), Sun et al, Immunity 28:52-63 (2008)). Similarly, IgG1b12 also has a long CDR3 loop and reacts with dsDNA (Haynes et al, Science 308:1906-1908 (2005), Saphire et al, Science 293:1155-1159 (2001)). These findings, coupled with the perceived rarity of clinical HIV-1 infection in patients with autoimmune disease (Palacios and Santos, Inter. J. STD AIDS 15:277-278 (2004)), have prompted the hypothesis that some species of broadly reactive neutralizing antibodies are not made due to downregulation by immune tolerance mechanisms (Haynes et al, Science 308:1906-1908 (2005), Haynes et al, Hum. Antibodies 14:59-67 (2005)). A corollary of this hypothesis is that some patients with autoimmune diseases may be “exposed and uninfected” subjects with some type of neutralizing antibody as a correlate of protection (Kay, Ann. Inter. Med. 111:158-167 (1989)). A patient with broadly neutralizing antibodies that target the 2F5 epitope region of the MPER of gp41 has been defined (Shen et al, J. Virol, 83:3617-25 (2009)).

The present invention results, at least in part, from the identification of cross-neutralizing plasma samples with high-titer anti-MPER peptide binding antibodies from among 156 chronically HIV-1-infected individuals. In order to establish if these antibodies were directly responsible for the observed is neutralization breadth, MPER-coated magnetic beads were used to deplete plasmas of these specific antibodies. Depletion of anti-MPER antibodies from a plasma sample from patient CAP206 resulted in a 68% decrease in the number of viruses neutralized. Antibodies eluted from the beads showed neutralization profiles similar to those of the original plasma, with potencies comparable to those of the known anti-MPER monoclonal antibodies (MAbs), 4E10, 2F5, and Z13e1. Mutational analysis of the MPER showed that the eluted antibodies had specificities distinct from those of the known MAbs, requiring a crucial residue at position 674.

The present invention provides MPER-specific cross-neutralizing antibodies (e.g., mAb 2311 from patient CAP206; mAb 2311 is also referred to herein as CAP206-CH12) and methods of using same.

### SUMMARY OF THE INVENTION

In general, the present invention relates to HIV-1 specific antibodies. More specifically, the invention relates to broadly neutralizing HIV-1 specific antibodies that target the gp41 MPER, and to methods of using same to both treat and prevent HIV-1 infection.

Objects and advantages of the present invention will be clear from the description that follows.

### BRIEF DESCRIPTION OF THE DRAWINGS

FIGS. 1A-1N. Evolution of an anti-MPR gp41 antibody response that mediates broad HIV-1 cross-neutralization.

FIGS. 2A-2F. (FIG. 2A) MPER-peptides for tetramers. (FIG. 2B) Development of broad neutralizing antibodies at 81 weeks after transmission in CAP206. (FIG. 2C) Dual MPER.03 tetramer staining on CAP206 memory B cells. (FIG. 2D) CDR regions of HIV-1 MPER MAbs 4E10 and CAP206_H2311. (FIG. 2E) Broad neutralizers-4E10 peptide surface. (FIG. 2F) Broadly-neutralizing IgG. 1=17b (−ve control); 2=JVB01WCK-01 (sample 713080258)—MPER+/2F5 peptide+/4E10+; 3=A8Y0B8F4-02 (sample 702010440)—MPER+/2F5 peptide++; 4=D770DX08-11 (sample 703010269)—MPER++++/4E10 peptide+++; 5=K89017D7-25 (sample 705010534)—MPER++/4E10 peptide+; 6=GC300WSN-19 (sample 707010175)—MPER+/−/2F5 peptide++; 7=JC300T05-16 (sample 707010536); 8=Vst. 208, Wk 16, 25/Jun./2008 (sample 707010219); 9=BC300WFK-04 (sample 707010457); 10=DC301 KVH-18 (sample 707010763).

FIGS. 3A and 3B. Adsorption of anti-MPER antibodies from plasmas BB34, BB81, and BB105. MAb 4E10 and plasma samples were adsorbed with MPER-peptide-coated beads or blank beads or left untreated. (FIG. 3A) All samples were assayed by ELISA for binding to the MPER or V3 peptide and tested for neutralization of the HIV-2-HIV-1 MPER chimera C1C. OD, optical density; cone, concentration. (FIG. 3B) Adsorbed plasmas were tested for neutralization of the HIV-1 envelope-pseudotyped viruses COT6.15, CAP206.8, and Du156.12.

FIGS. 4A and 4B. Antibodies eluted from MPER-coated beads contain cross-neutralizing activity. (FIG. 4A) Neutralization of C1C by eluates from MPERcoated beads of plasmas BB34, CAP206, and SAC21 and MAbs 4E10, Z13e1, and 2F5. cone, concentration. (FIG. 4B) Neutralization of HIV-1 subtype C envelope-pseudotyped viruses COT6.15, ZM197M.PB7, Du 156.12, and CAP206.8 and subtype B TRO.11 and JR-FL.

FIG. 5. Comparison of the IgG subclass profiles between original plasmas and eluates from MPER-coated beads. The pie charts represent the IgG subclasses found in the BB34, CAP206, and SAC21 plasmas and eluates. The table shows the IgG subclass concentrations in plasmas and in eluates, b.d, below detection level.

FIGS. 6A-6D. Neutralizing anti-MPER antibodies are IgG3 in BB34 but not in CAP206. (FIGS. 6A and 6B) IgG subclass profiles of total IgG, FTpA, and EpA of BB34 (A) and CAP206 (B). (FIG. 6C) BB34 fractions were tested for neutralization of C1C and HIV-1 envelope-pseudotyped viruses, as well as binding to the MPER peptide in ELISA. OD, optical density; cone, concentration. (FIG. 6D) CAP206 fractions were tested for neutralization of C1C and HIV-1 envelope-pseudotyped viruses.

FIG. 7. Antigen-specific staining of memory B cells from CAP206. Flow cytometric plot of CD19+/CD27+memory B cells from CAP206 stained with labeled MPR.03 tetramers. Circled cells represent double-positive memory B cells that were single-cell sorted into 96-well plates.

FIGS. 8A-8F. Specificity, avidity and lack of lipid binding of CAP206-CH12 mAb. (FIG. 8A) ELISA showing specific binding of CAP206-CH12 to MPR.03 and MPER656 peptides. A scrambled MPR.03 peptide was negative as were peptides to the gp41 immunodominant region (SP400), 2F5 epitope (SP62 peptide) and 4E10 epitope. There was also no binding to JRFL gp140, ConS gp140 or gp41 (FIG. 8B) Surface Plasmon Resonance (SPR) showing on-off rates of CAP206-CH12 to MPR.03 peptide compared to 2F5 and 4E10 (FIGS. 8C and 8D) SPR showing on-off rates of CAP206-CH12 and its RUA to MPR.03 peptide (FIG. 8E) lack of binding of CAP206-CH12 to cardiolipin compared to 4E10 and (FIG. 8F) Inability of CAP206-CH12 to bind MPER 656 peptide embedded in liposomes.

FIG. 9. Polyspecificity of CAP206-CH12 and its RUA.

FIG. 10. MPER sequences of viruses sensitive and resistant to CAP206-CH12 mAb. Amino acids at positions 674 and 677—the nominal epitope of this mAb are highlighted.

FIG. 11. VH and VL sequences of CAP206-CH12 and CAP206-CH12 RUA.

FIGS. 12A and 12B. (FIG. 12A) 2311 mAb. (FIG. 12B) 4E10 mAb.

FIG. 13. Schematic diagram for generation of linear full-length Ig heavy- and light-chain genes. Shown is a schematic diagram for the assembly by overlapping PCR of linear full-length Ig heavy-chain gene (A), Ig kappa light-chain gene (B) and lambda light-chain gene (C) expression cassettes. Sequences in the Ig leader region at the 3′ end of the C fragment overlapping with the sequences at the 5′ end of the V_{H}, V_{κ} and V_{λ} fragments are indicated. Sequences at the 5′ end of the H fragment, K fragment and L fragments overlapping with the sequences at the 3′ end of the corresponding V_{H}, V_{κ} and V_{λ} fragments are also indicated. The same forward and reverse primers (CMV-F262 and BGH-R1235, Supplementary Table 7) used in the overlapping PCR for all Ig heavy-chain genes, Ig kappa light-chain genes and lambda light-chain genes are indicated with arrows.

FIGS. 14A-14D. Expression of synthetic 2F5 V_{H} and V_{L} n panel A, lane M: DNA ladders marked in kilobases (kb) next to the lane, lanes 1-8, respectively: DNA fragments C (705 bp), H (1,188 bp), K (569 bp), synthetic 2F5 V_{H} (489 bp) and V_{L} (370 bp) as well as the full-length Ig heavy- (2339 bp) and is light-chain (1595 bp) gene expression cassettes generated by PCR and analyzed on a 1% agarose gel. Arrows indicate the expected DNA fragments. Panel B shows the results of Western blots of commercial mAb 2F5 (lane 1), supernatant harvested from 293T cells transfected with plasmids expressing synthetic 2F5 Ig genes (lane 2), with the linear full-length synthetic 2F5 Ig heavy- and light-chain gene cassettes (lanes 3) and mock-transfected 293T cells (lane 4). Igs on the blots were detected by either an anti-human heavy-chain specific antibody or an anti-human kappa light-chain specific antibody as indicated at the bottom of the blots. The arrows with short notations indicate the possible composition of antibody heavy-chains (HC) and light-chain (LC). Panel C is a comparison of the amounts of Ig secreted from 293T cells transiently transfected with either linear full-length synthetic 2F5 heavy- and light-chain Ig gene constructs (1 μg of each) or plasmids (1 μg of each) expressing the synthetic 2F5 heavy- or light-chain Ig genes as indicated. Average amounts (n=6) of IgG secreted in the transfected 293T cells are shown on the y-axis and were determined by comparison to a standard curve generated using known concentrations of IgG 1, Panel D shows the measurement of antibody binding by ELISA to the following antigens: HIV-1 Env MPER epitope peptide SP62, HIV-1 gp41 and HIV-1 gp140 or scrambled SP62 peptides as negative controls. Supernatants from the 293T cells transfected with each DNA construct (indicated on the x-axis) were assayed by ELISA for binding to HIV-1 antigens and the results were compared to mAb 2F5 at 1.25 μg/ml.

FIGS. 15A and 15B. Measurement of the reactivity of recombinant antibodies. Panel A compares the reactivity of recombinant antibody produced in 293T cells by transfection of 7B2 Ig genes and mAb 7B2 produced by the EBV-transformed 7B2 B cell line. Supernatants of 293T cells transfected with linear rH70 Ig genes and supernatants from mock transfections were used as negative controls. Panel B compares the reactivity of the recombinant antibody produced by transfection with linear 08 Ig genes with antibody produced by the EBV-transformed G8 B cell line and with purified mAb G8 supernatant from 293T cells transfected with linear rH0045 Ig genes with unknown specificity and from mock-transfection were used as negative controls.

FIG. 16. Changes in plasmablast populations induced by vaccination with Fluzone. Shown is the frequency of plasmablasts (located in the upper right and defined as CD19⁺, CD20 low-neg, CD3⁻, CD14⁻, CD16⁻, CD235⁻, CD38^{hl} and CD27^{hi}) in PB collected at day 0, 7 and 21 after vaccination from a subject vaccinated with Fluzone® 2007-2008 and analyzed by flow cytometry.

FIGS. 17A-17C. Reactivity of recombinant human mAbs from single plasma cells after Fluzone® 2007-2008 vaccination. Shown are the results of ELISA assays for detection of the reactivity of the antibodies derived from the individual Ig heavy- and light-chain gene pairs isolated from sorted single plasma cells (first 9 bars in the x-axis) or a negative control Ig pair (-Ab Ctl) and mock transfection control (Mock Tx.). Antibody reactivity to the inactivated influenza viruses (Panel A), with H1 A/Solomon islands HA (Panel B) and H3 A/Wisconsin HA (Panel C) are shown. Serum samples collected from the vaccinee at day 0 and 21 (grey columns) were used as positive controls in these assays. Data are representative of two independent experiments.

FIG. 18. Serum antibody responses to Fluzone vaccination. Serum samples were collected from the vaccinee at day 0 and 21 days after vaccination with Fluzone and assayed against a panel of influenza antigens as indicated on the x-axis. Shown is the reactivity of serum samples at 1:800 dilution to the indicated antigens. Data are representative two independent experiments.

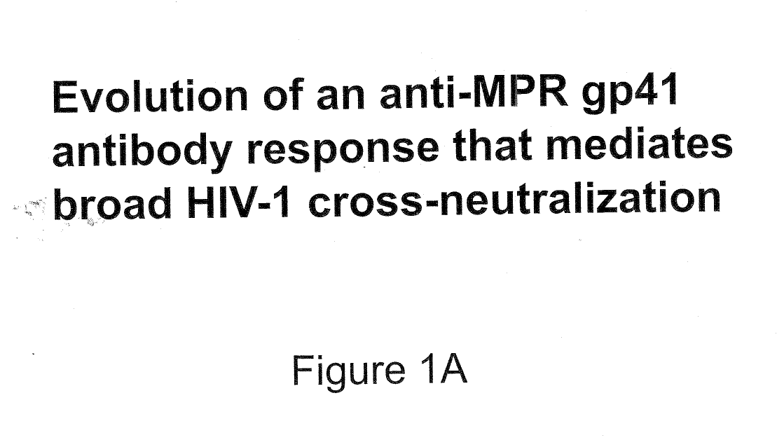

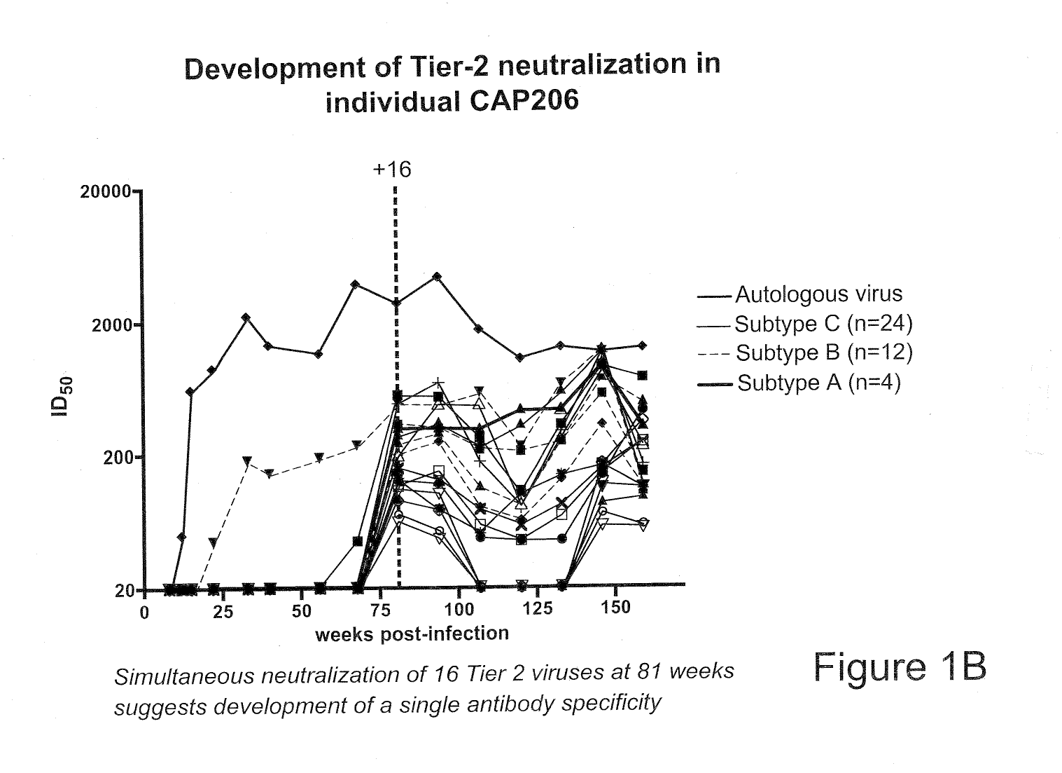

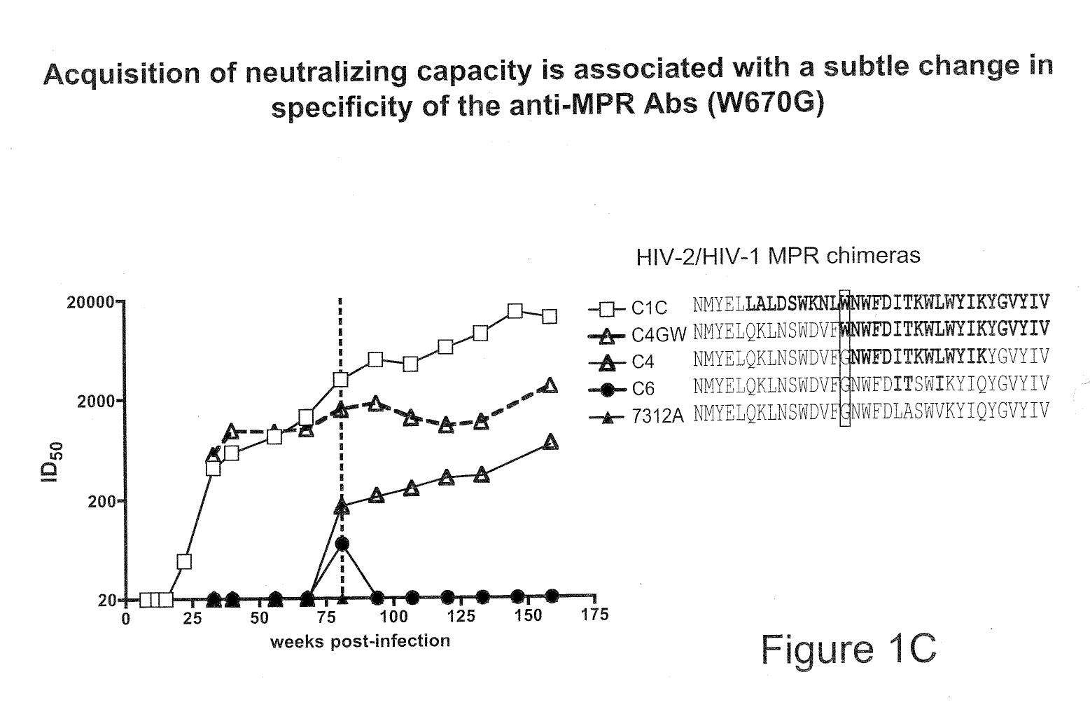

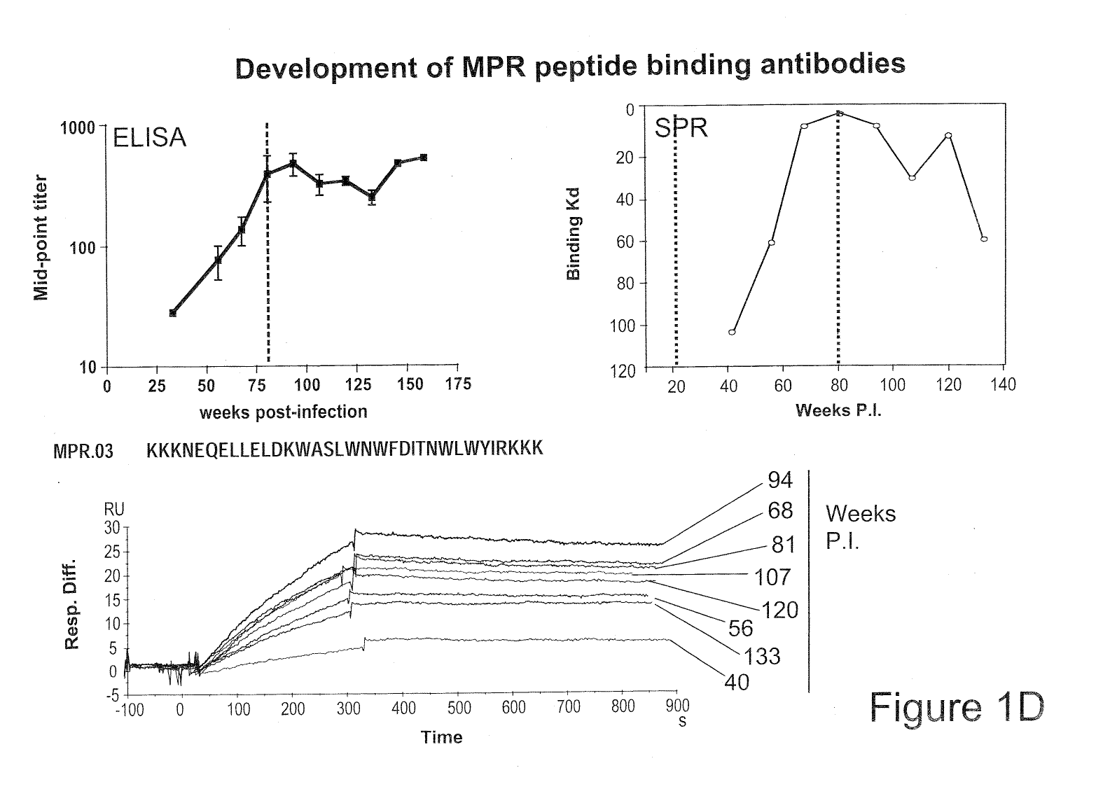

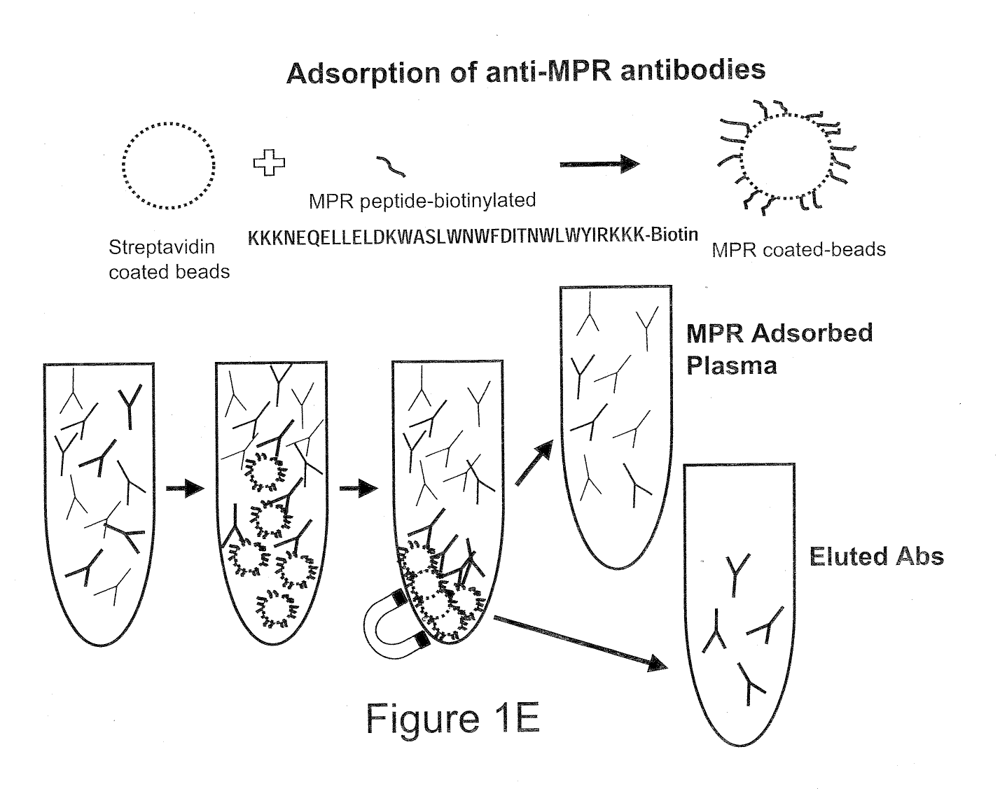

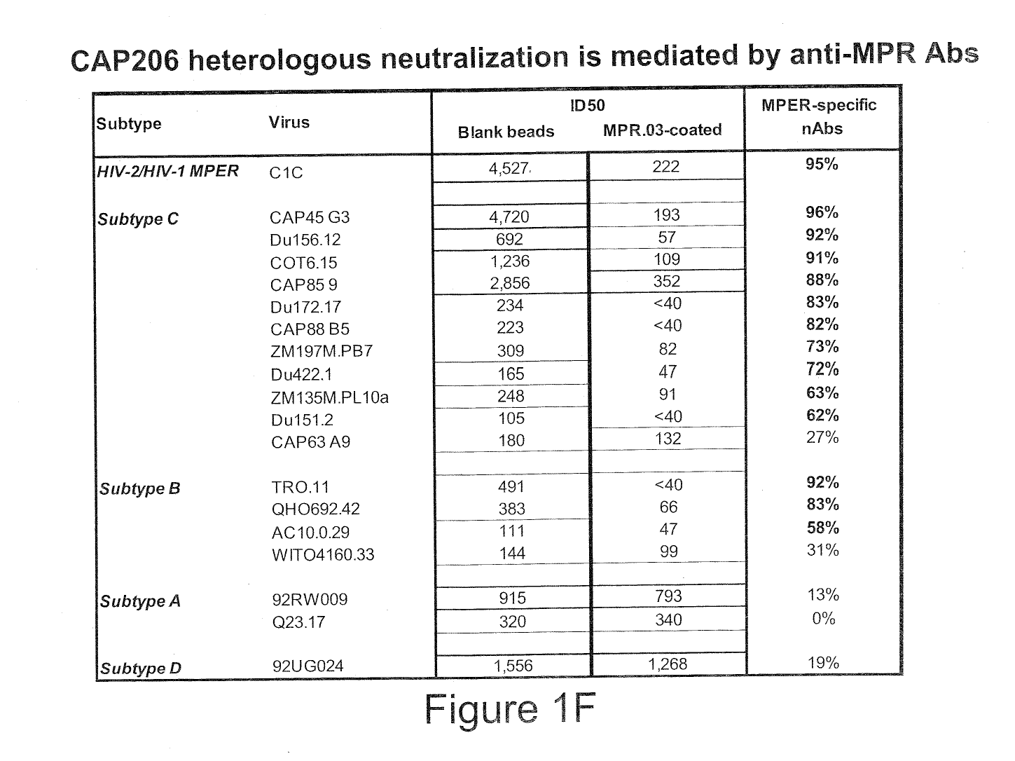

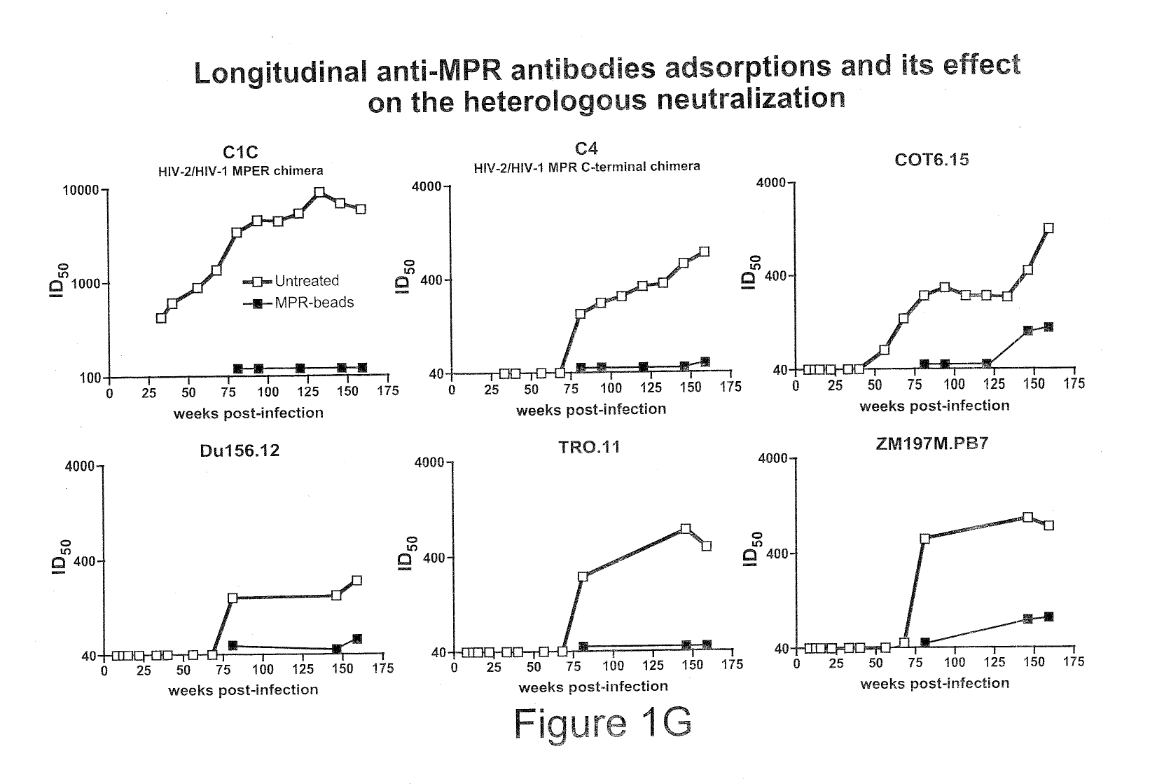

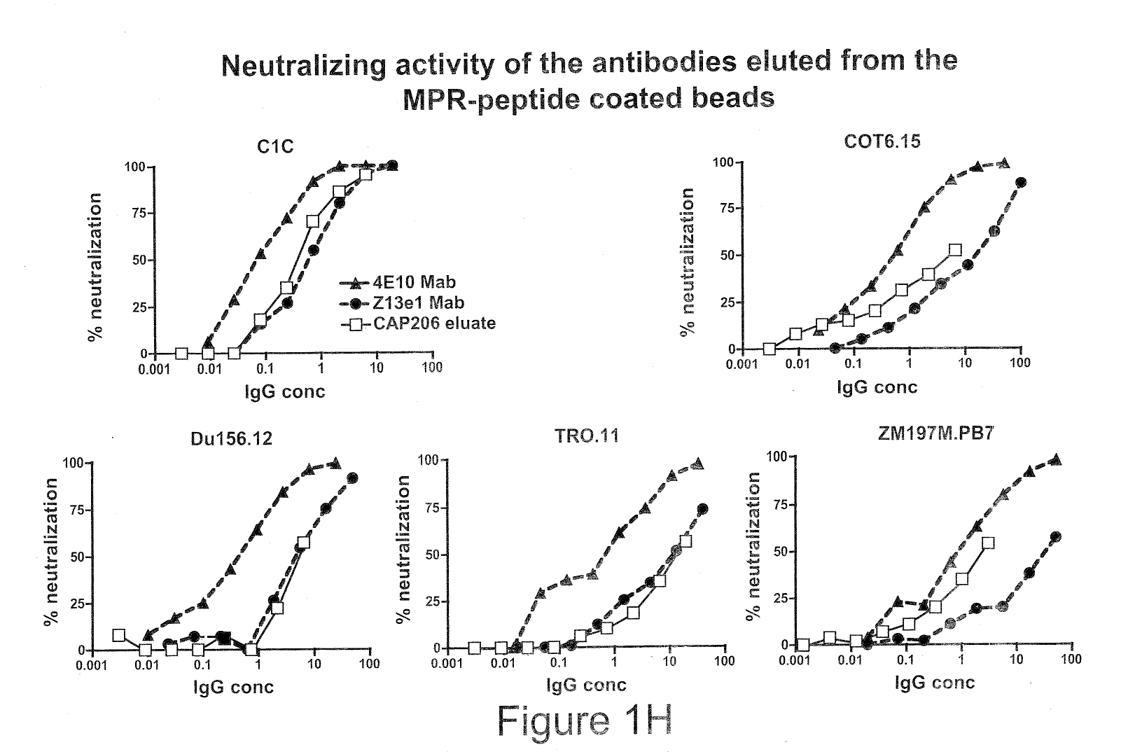

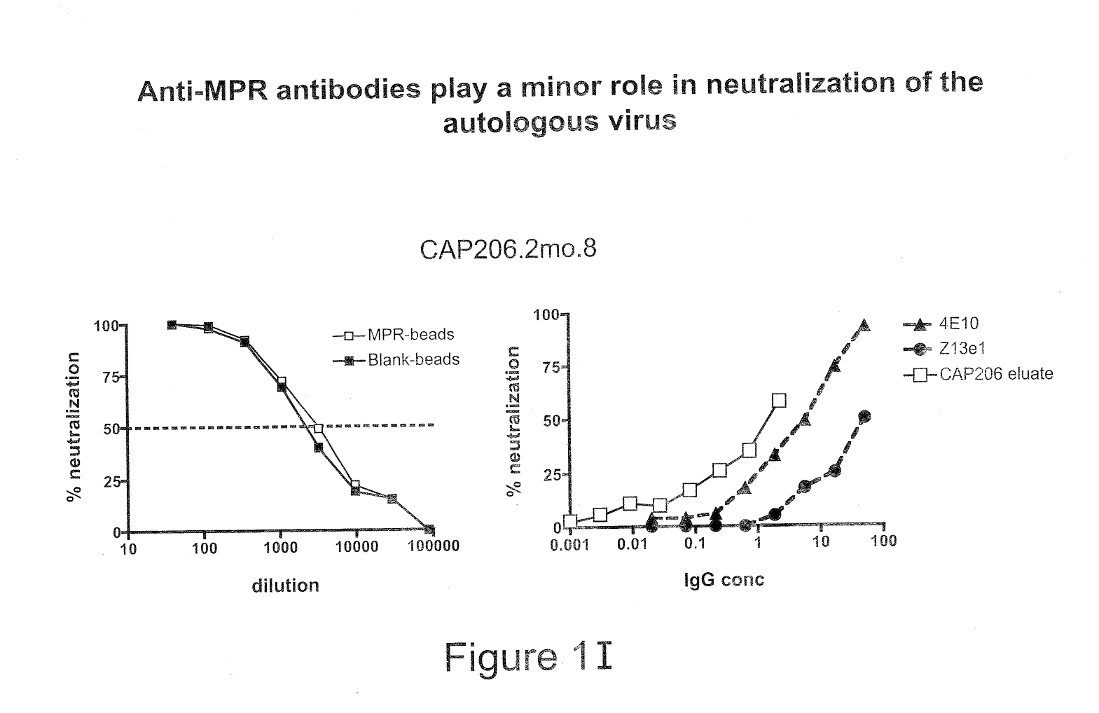

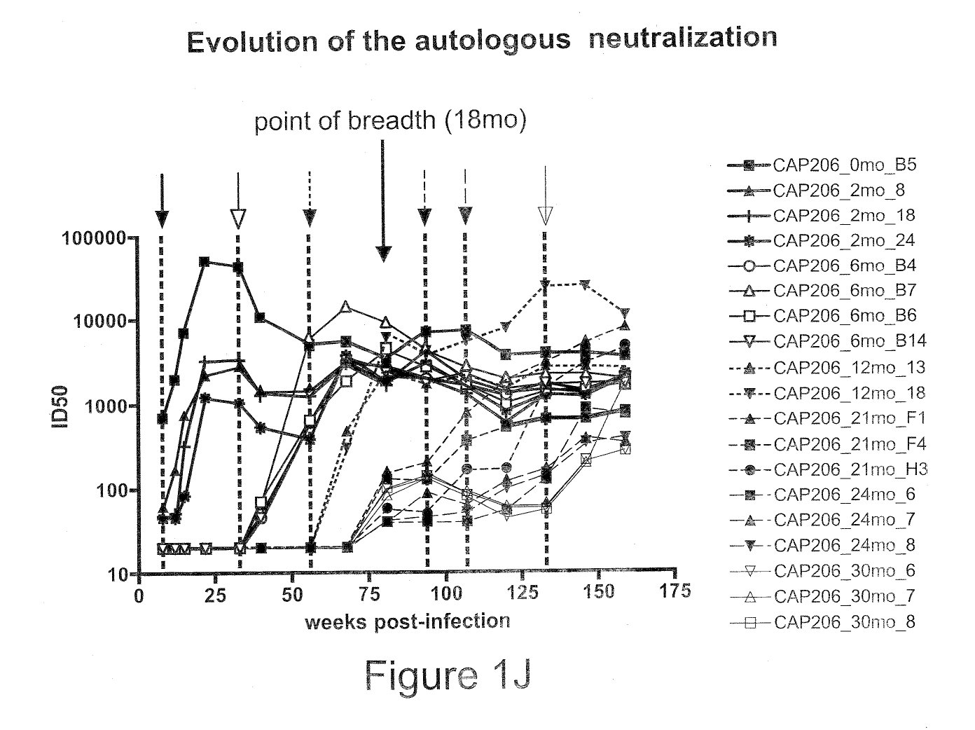

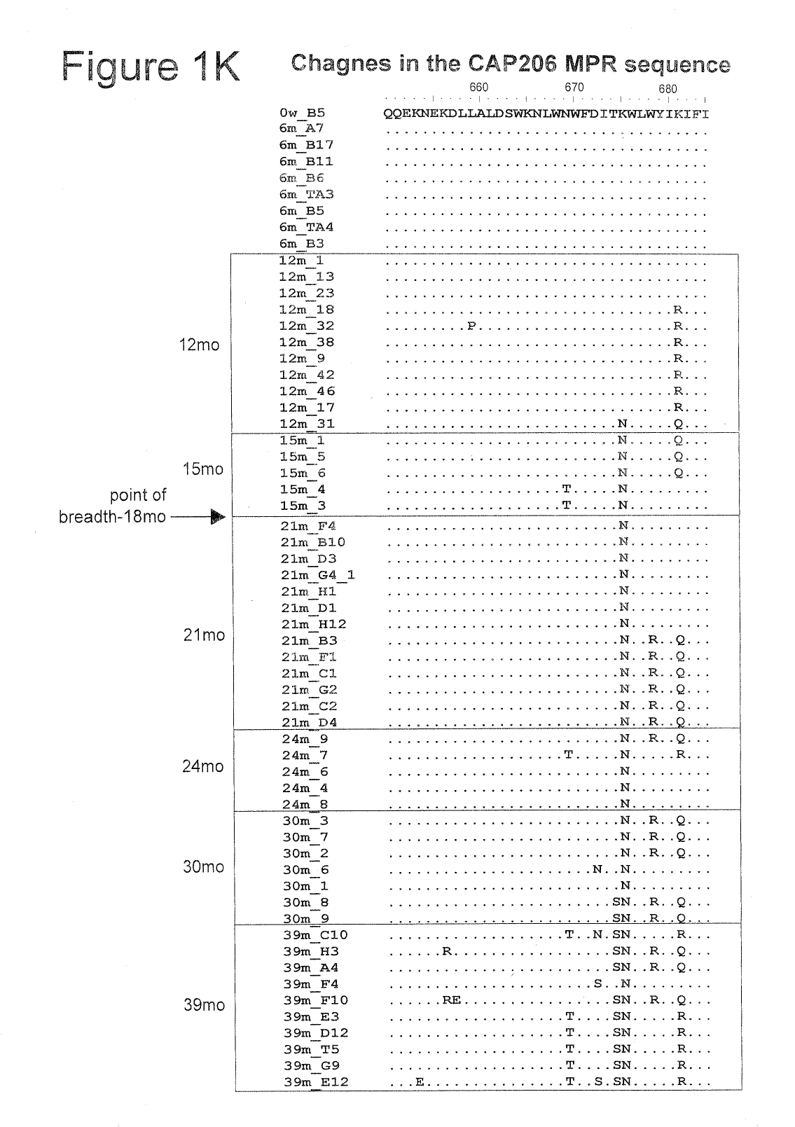

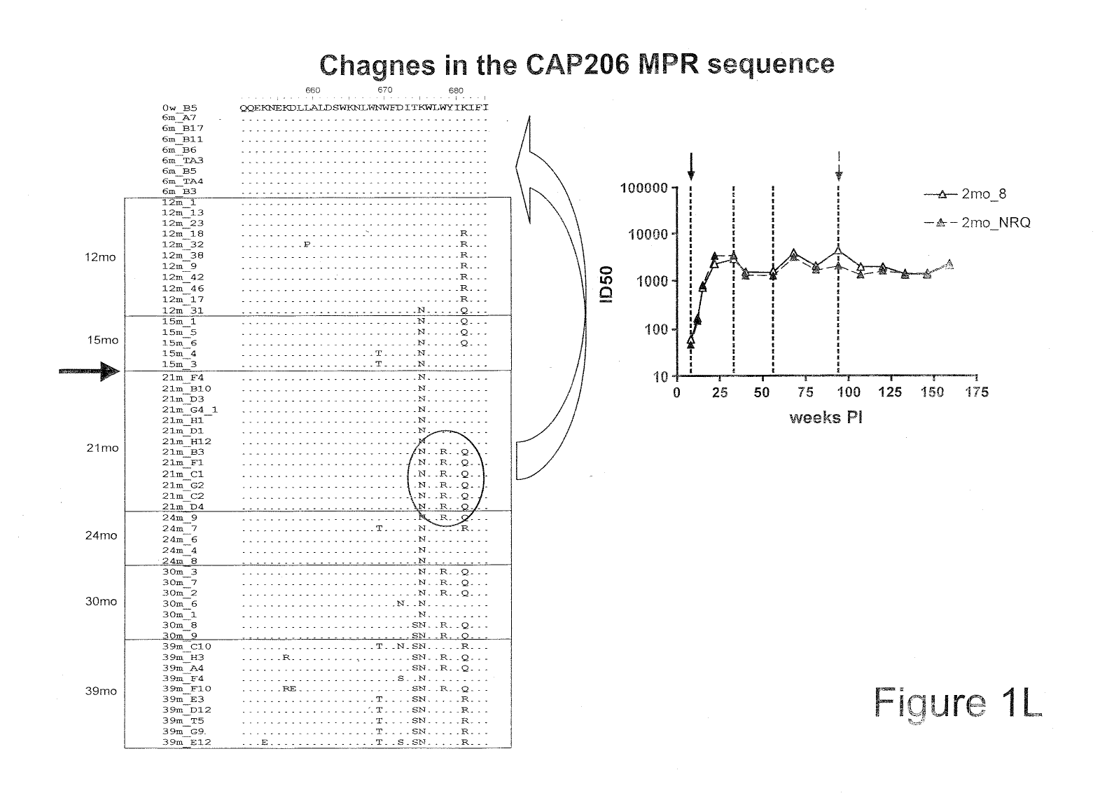

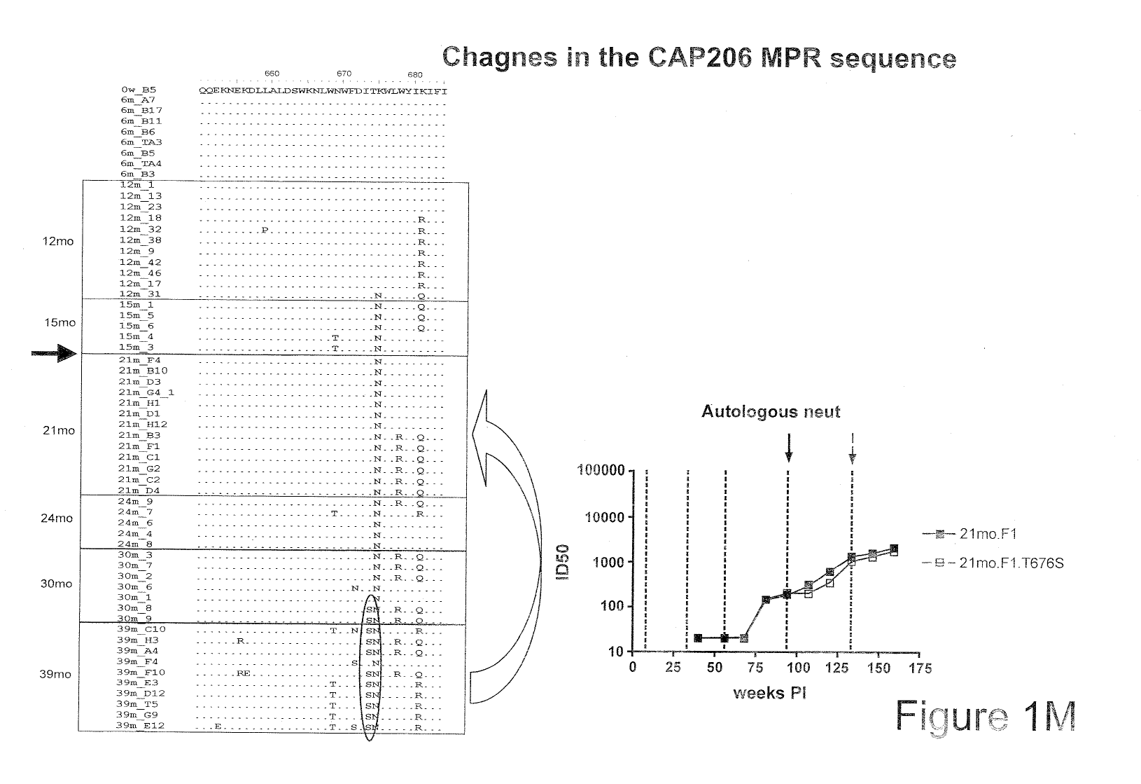

### DETAILED DESCRIPTION OF THE INVENTION

The present invention relates, in one embodiment, to a method of inhibiting infection of cells (e.g., T-cells) of a subject by HIV-1. The invention also relates to a method of controlling the initial viral load and preserving the CD4+ T cell pool and preventing CD4+ T cell destruction. The method comprises administering to the subject (e.g., a human subject) an HIV-1 specific antibody that binds the distal region of the HIV-1 Env gp41MPER around the FDI in the sequence NEQELLELDKWASLWNWFDITNWLWY, or fragment thereof, in an amount and under conditions such that the antibody, or fragment thereof, inhibits infection.

In accordance with the invention, the antibodies can be administered prior to contact of the subject or the subject's immune system/cells with HIV-1 or after infection of vulnerable cells. Administration prior to contact or shortly thereafter can maximize inhibition of infection of vulnerable cells of the subject (e.g., T-cells).

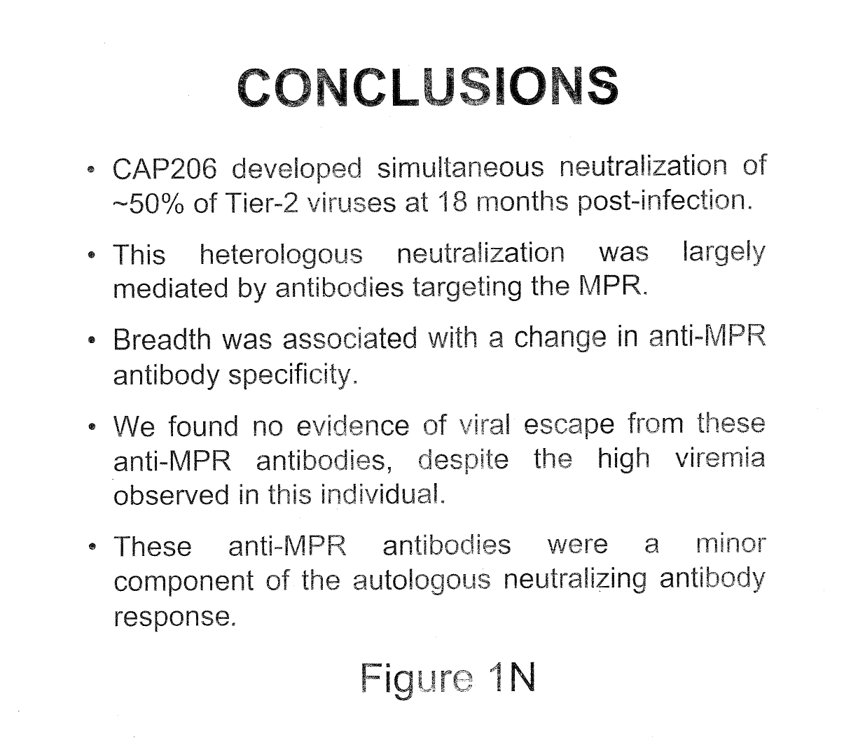

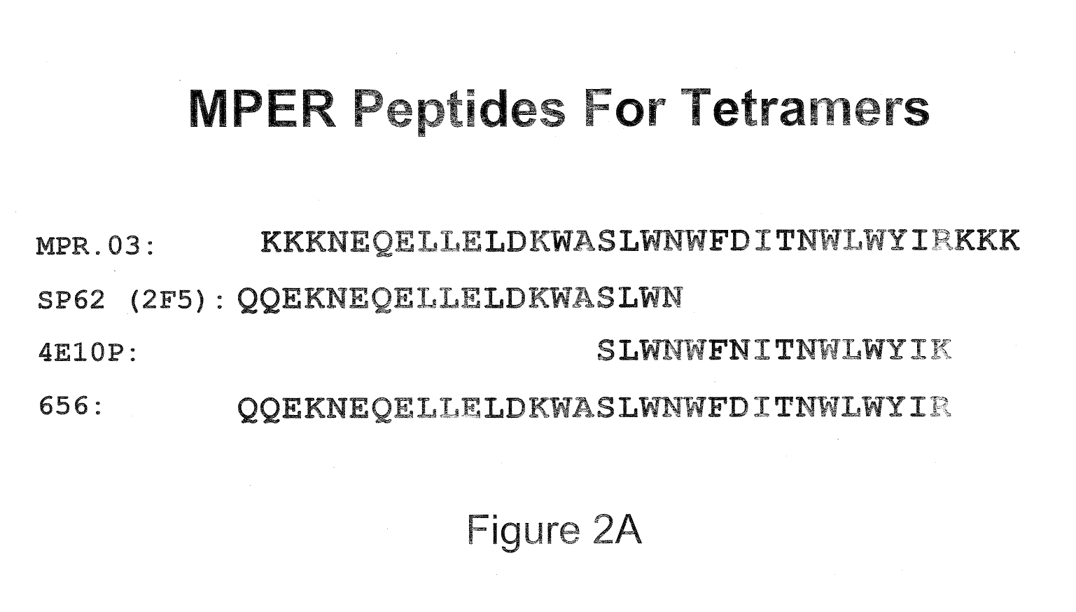

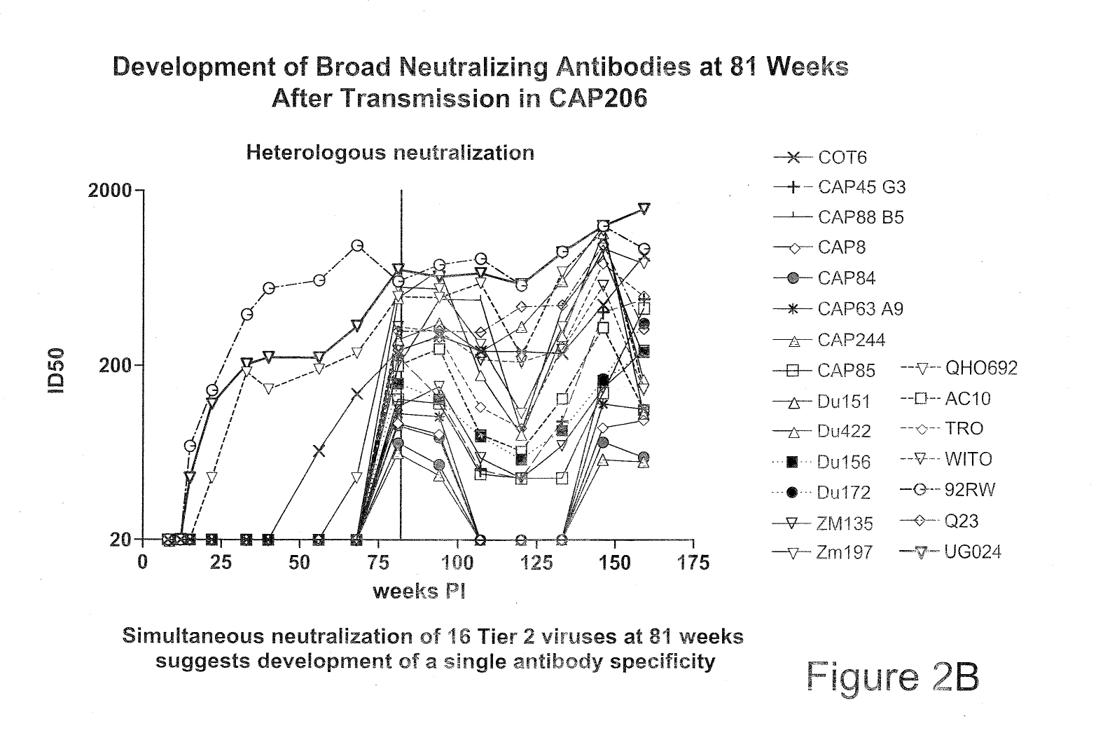

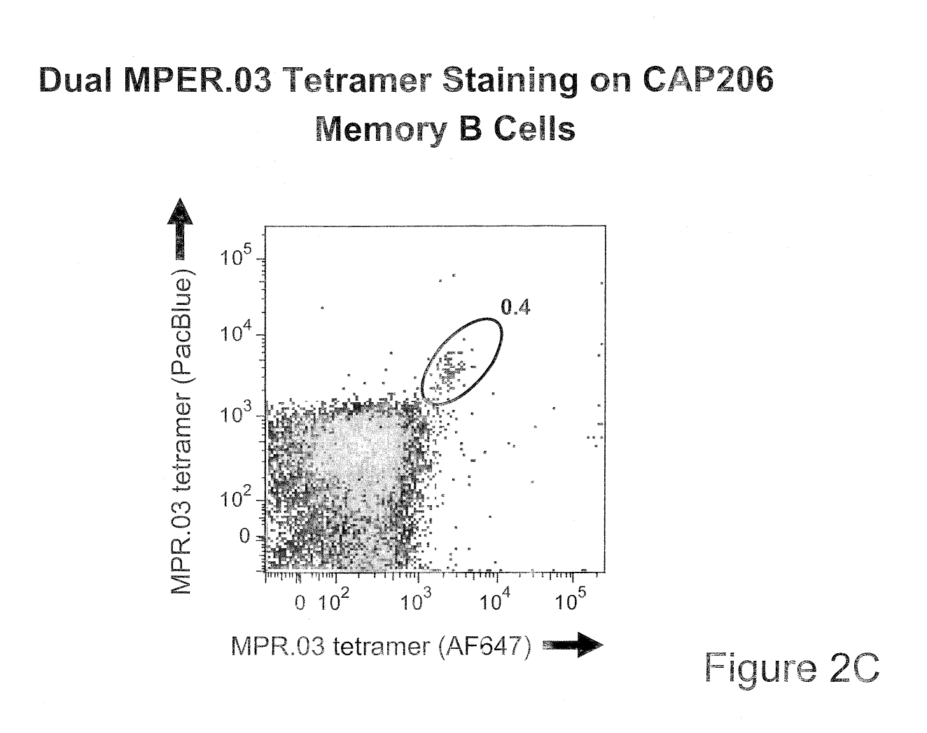

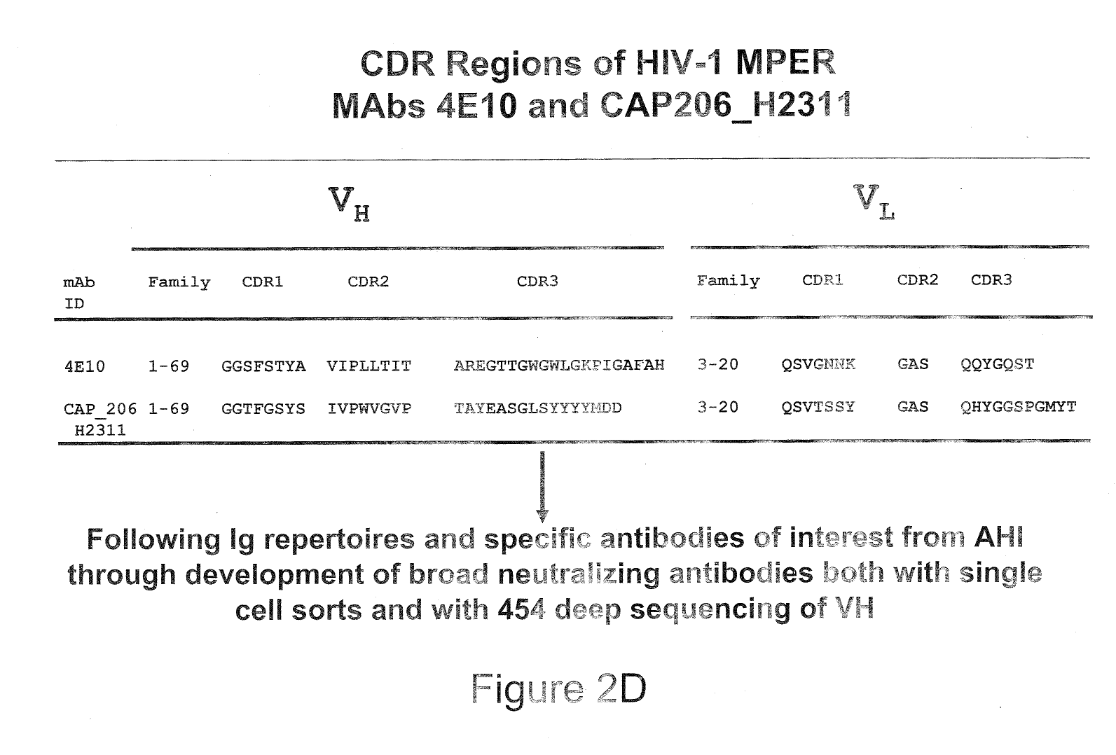

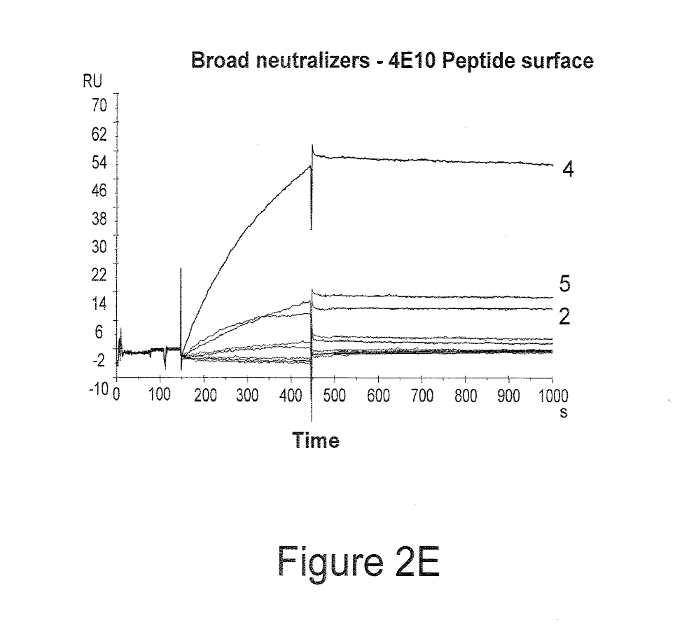

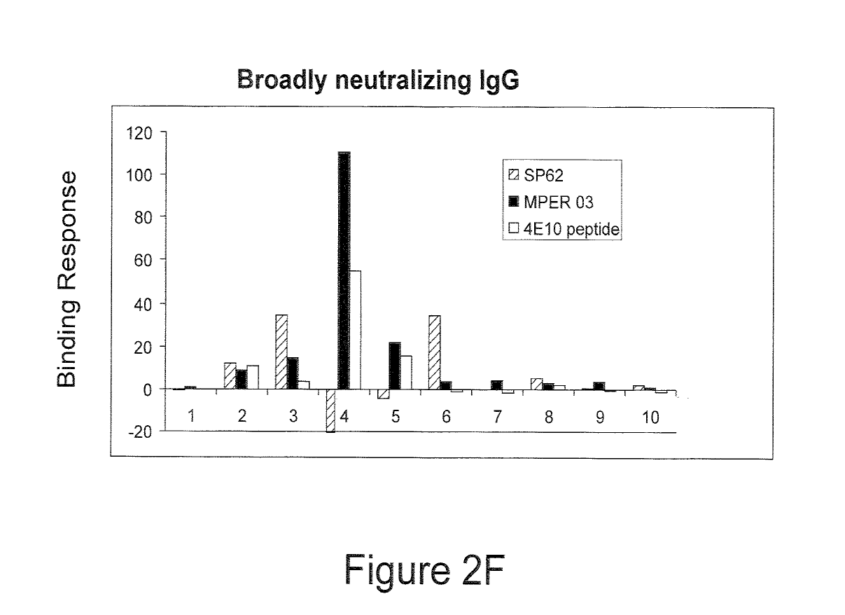

One preferred antibody for use in the invention is a mAb having the variable heavy and variable light sequences of the 2311 antibody as set forth in Table 1 (see also FIG. 11) or fragment thereof. The invention also includes antibodies or fragments thereof comprising a heavy chain and a light chain wherein the heavy chain variable region sequence comprises V_{H} CDR1, CDR2 and CDR3 shown in FIG. 2D for CAP_{—}206 H2311 (CAP206-CH12) and the light chain variable region sequence comprises V_{I}, CDR1, CDR2 and CDR3 shown in FIG. 2D (see also FIG. 11) for CAP_{—}206-CH12.

TABLE 1
>CAP_2311 HC.seq
CAGGTGCAGCTGGTGCAGTCTGGGGCGGAAGTGAAGAAGCCTGGGTCCTCGGTGAAGCTCTCCTG
TAAGGCTTCTGGAGGCACCTTCGGCAGCTATTCTGTCACCTGGGTGCGCCAGGCCCCTGGACAAA
CGTTTGAGTGGGTGGGCAGGATCGTCCCTTGGGTTGGTGTTCCGAACTACGCACCGAAGTTCCAG
GGCAGAGTCACCATTACCGCGGACAAATCGAGCACAGTCTACATGGAATTGACCAGTCTGAGATT
TGAGGACACGGCCGTCTATTACTGTGCGACAGCCTATGAGGCGAGTGGGTTGTCATACTACTACT
ACATGGACGACTGGGGCAAAGGGACCACGGTCACCGTCTCCTCAGCCTCCACCAAGGGCCCATCG
GTCTTCCCCCTGGCACCCTCCTCCAAGAGCACCTCTGGGGCAAAAAAGGGGCCAAAGCGGGGGAA
ACCCCCAGGAGC
>CAP_2311 HC.pep
QVQLVQSGAEVKKPGSSVKLSCKASGGTFGSYSVTWVRQAPGQTFEWVGRIVPWVGVPNYAPKFQ
GRVTITADKSSTVYMELTSLRFEDTAVYYCATAYEASGLSYYYYMDDWGKGTTVTVSS
>CAP_2311 LC.seq
GAAATTGTGTTGACGCAGTCTCCAGGCACCCTGTCTTTGTCTCCAGGGGAAAGAGCCACCCTCTC
CTGCAGGGCCAGTCAGAGTGTTACCAGCAGCTACTTAGCCTGGTTCCGGCACAAGCCTGGCCAGG
CTCCAAGGCTCCTCATATATGGTGCATCATACAGGGGCACTGGCATTCCAGACAGAATCAGTGGC
AGTGGGTCTGGGACAGACTTCACTCTCACCATCAGCAGACTGGAGCCTGAAGATTTTGCAGTGTA
TTACTGTCAGCACTATGGTGGCTCACCTGGGATGTACACTTTTGGCCAGGGGACCAGGCTGGAGA
TCAAA
>CAP_2311 LC.pep
EIVLTQSPGTLSLSPGERATLSCRASQSVTSSYLAWFRHKPGQAPRLLIYGASYRGTGIPDRISG
SGSGTDFTLTISRLEPEDFAVYYCQHYGGSPGMYTFGQGTRLEIK

As indicated above, either the intact antibody or fragment (e.g., antigen binding fragment) thereof can be used in the method of the present invention. Exemplary functional fragments (regions) include scFv, Fv, Fab′, Fab and F(ab′)₂ fragments. Single chain antibodies can also be used. Techniques for preparing suitable fragments and single chain antibodies are well known in the art. (See, for example, U.S. Pat. Nos. 5,855,866; 5,877,289; 5,965,132; 6,093,399; 6,261,535; 6,004,555; 7,417,125 and 7,078,491 and WO 98/45331.) The invention also includes variants of the antibodies (and fragments) disclosed herein, including variants that retain the binding properties of the antibodies (and fragments) specifically disclosed, and methods of using same in the present method. For example, the invention includes an isolated human antibody or fragment thereof that binds selectively to gp41MPER and that comprises 2, 3, 4, 5 or 6 CDRs as set forth in FIG. 2D for CAP-CH12 (see also FIG. 11). Modifications of mAb 2311 (CAP206-CH12) that can be used therapeutically in accordance with the invention include IgA, IgM and IgG1, 2, 3 or 4 versions of mAb 2311 (CAP206-CH12) VH and VL chains.

The antibodies, and fragments thereof, described above can be formulated as a composition (e.g., a pharmaceutical composition). Suitable compositions can comprise the antibody (or antibody fragment) dissolved or dispersed in a pharmaceutically acceptable carrier (e.g., an aqueous medium). The compositions can be sterile and can in an injectable form. The antibodies (and fragments thereof) can also be formulated as a composition appropriate for topical administration to the skin or mucosa. Such compositions can take the form of liquids, ointments, creams, gels, pastes or aerosols. Standard formulation techniques can be used in preparing suitable compositions. The antibodies can be formulated so as to be administered as a post-coital douche or with a condom.

The antibodies and antibody fragments of the invention show their utility for prophylaxis in, for example, the following settings:

i) in the setting of anticipated known exposure to HIV-1 infection, the antibodies described herein (or binding fragments thereof) can be administered prophylactically (e.g., IV or topically) as a microbiocide,

ii) in the setting of known or suspected exposure, such as occurs in the setting of rape victims, or commercial sex workers, or in any heterosexual transmission with out condom protection, the antibodies described herein (or fragments thereof) can be administered as post-exposure prophylaxis, e.g., IV or topically,

iii) in the setting of Acute HIV infection (AHI), antibodies described herein (or binding fragments thereof) can be administered as a treatment for AHI to control the initial viral load and preserve the CD4+ T cell pool and prevent CD4+ T cell destruction, and

iv) in the setting of maternal to baby transmission while the child is breastfeeding.

Suitable dose ranges can depend, for example, on the antibody and on the nature of the formulation and route of administration. Optimum doses can be determined by one skilled in the art without undue experimentation. Doses of antibodies in the range of 10 ng to 20 μg/ml can be suitable.

The present invention also includes nucleic acid sequences encoding the antibodies, or fragments thereof, described herein. The nucleic acid sequences can be present in an expression vector operably linked to a promoter. The invention further relates to isolated cells comprising such a vector and to a method of making the antibodies, or fragments thereof, comprising culturing such cells under conditions such that the nucleic acid sequence is expressed and the antibody, or fragment, is produced.

Certain aspects of the invention can be described in greater detail in the non-limiting Examples that follows. (See also Shen et al, J. Virol, 83(8):3617-25 Epub 2009, Zhu and Dimitrov, Methods Mol. Boil. 525:129-142 (2009), Dimitrov and Marks, Methods Mol. Biol. 525:1-27 (2009), Zhang et al, J. Virol. 82(14):6869-6879 (2008), Prabakaran et al, Advances in Pharmacology 55:33-97 (2007), Gray et al, J. Virol 83:8925-8937 (2009), Liao et al, J. Virol. Methods 158:171-179 (2009)).

### Example 1

### Experimental Details

Plasma Samples and Viruses.

Plasmas BB34, BB81, BB105, and SAC21 were from HIV-1-infected blood donors identified by the South African National Blood Service in Johannesburg. The BB samples were collected between 2002 and 2003 and have been described previously (Binley et al, J. Virol. 82:11651-11668 (2008), Gray et al, J. Virol. 83:8925-8937 (2009)). The SAC plasma samples are from a second blood donor cohort that was assembled using a similar approach. Briefly, aliquots from 105 HIV-1-infected blood donations made between 2005 and 2007 were screened in the BED assay to eliminate 29 incident infections. Eight samples neutralized the vesicular stomatitis virus G control pseudovirus and were excluded. SAC21 was among the remaining 68 aliquots that were tested against three subtype B and three subtype C primary viruses to identify those with neutralization breadth. The plasma sample CAP206 corresponded to the 3-year visit of an individual in the Centre for the AIDS Programme of Research in South Africa (CAPRISA) cohort (Gray et al, J. Virol. 81:6187-6196 (2007), van Loggerenberg et al, PLoS ONE 3:e1954 (2008)). The envelope genes used to generate pseudovirus were either previously cloned (Gray et al, J. Virol. 81:6187-6196 (2007)) or obtained from the NIH AIDS Research and Reference Reagent Program or the Programme EVA Centre for AIDS Reagents, National Institute for Biological Standards and Control, United Kingdom. The HIV-2 7312A and derived MPER chimeras were obtained from George Shaw (University of Alabama, Birmingham).

Neutralization Assays.

Neutralization was measured as a reduction in luciferase gene expression after a single-round infection of JC53b1-13 cells, also known as TZM-b1 cells (NIH AIDS Research and Reference Reagent Program; catalog no. 8129) with Env-pseudotyped viruses (Montefiori, D. C., Evaluation neutralizing antibodies against HIV, SIV and SHIV in luciferase reporter gene assays, p. 12.1-12.15 (2004), Coligan et al (ed.), Current protocols in immunology, John Wiley & Sons, Hoboken, NJ17). Titers were calculated as the 50% inhibitory concentration (IC50) or the reciprocal plasma/serum dilution causing 50% reduction of relative light units with respect to the virus control wells (untreated virus) (ID50). Anti-MPER specific activity was measured using the HIV-2 7312A and the HIV-2/HIV-1 MPER chimeric constructs (Gray et al, J. Viral. 81:6187-6196 (2007)). Titers threefold above background (i.e., the titer against 7312A) were considered positive.

Serum Adsorption and Elution of Anti-MPER Antibodies.

Streptavidin-coated magnetic beads (Dynal MyOne Streptavidin C1; Invitrogen) were incubated with the biotinylated peptide MPR.03 (KKKNEQELLELDKWASLWNWFDITNW LWYIRKKK-biotin-NH2) (NMI, Reutlingen, Germany) at a ratio of 1 mg of beads per 20 μg peptide at room temperature for 30 min. Plasmas were diluted 1:20 in Dulbecco's modified Eagle's medium (DMEM)-10% fetal bovine serum and incubated with the coated beads for 1 h at a ratio of 2.5 mg of coated beads per ml of diluted plasma. This was followed by a second adsorption at a ratio of 1.25 mg of coated beads per ml of diluted sample. After each adsorption, the beads were removed with a magnet, followed by centrifugation, and were stored at 4° C. The antibodies bound to the beads were eluted by incubation with 100 mM glycine-HCl elution buffer (pH 2.7) for 30 s with shaking and then pelleted by centrifugation and held in place with a magnet. The separated immunoglobulin G (IgG) was removed and placed into a separate tube, where the pH was adjusted to between 7.0 and 7.4 with 1 M Tris (pH 9.0) buffer. The same beads were acid eluted twice more. The pooled eluates were then diluted in DMEM, washed over a 10-kDa Centricon plus filter, and resuspended in DMEM. Antibody concentrations were determined using an in-house total-IgG quantification enzyme-linked immunosorbent assay (ELISA) as described below. The adsorbed sera were then used in ELISAs and neutralization assays.

MPER-Peptide ELISA.

Synthetic MPR.03 peptide or V3 peptide (TRPGNN TRKSIRIGPGQTFFATGDIIGDIREA11) was immobilized at 4 μg/ml in a 96-well high-binding ELISA plate in phosphate-buffered saline (PBS) overnight at 4° C. The plates were washed four times in PBS-0.05% Tween 20 and blocked with 5% skim milk in PBS-0.05% Tween 20 (dilution buffer). Adsorbed plasmas, as well as control samples, were serially diluted in dilution buffer and added to the plate for 1 h at 37° C. Bound antibodies were detected using a total antihuman IgG-horseradish peroxidase conjugate (Sigma-Aldrich, St. Louis, Mo.) and developed using TMB substrate (Thermo, Rockford, Ill.). The plates were read at 450 nm on a microplate reader.

IgG Quantification ELISA.

Goat anti-human IgG antibody was immobilized in a 96-well high-binding plate in carbonate-bicarbonate buffer overnight at 4 μg/ml. The plates were washed four times in PBS-0.05% Tween 20 and blocked with 5% goat serum, 5% skim milk in PBS-0.05% Tween 20. The eluted antibodies were serially diluted and added to the plate for 1 h at 37° C. The bound IgG was detected using a total anti-human IgG-horseradish peroxidase conjugate (Sigma-Aldrich) as described above.

IgG Subclass Fractionation.

Total IgG was extracted from plasma samples using a protein G column (NAb Protein G Spin Kit; Thermo). The IgG3 fraction was separated from the other IgG subclasses using a protein A column (NAb Protein A Spin Kit; Thermo). Protein G and protein A flowthrough fractions and eluted IgGs were tested using a Human IgG Subclass Profile ELISA Kit (Invitrogen Corporation, Carlsbad, Calif.). The concentration of each IgG subclass was calculated relative to a subclass-specific standard curve provided by the manufacturer.

Site-Directed Mutagenesis.

Specific amino acid changes in the MPER of the envelope clone COT6.15 (Gray et al, PLoS Med. 3:e255 (2006)) were introduced using the QuikChange Site Directed Mutagenesis Kit (Stratagene, La Jolla, Calif.). Mutations were confirmed by sequence analysis.

### Results

Adsorption of Anti-MPER Antibodies.

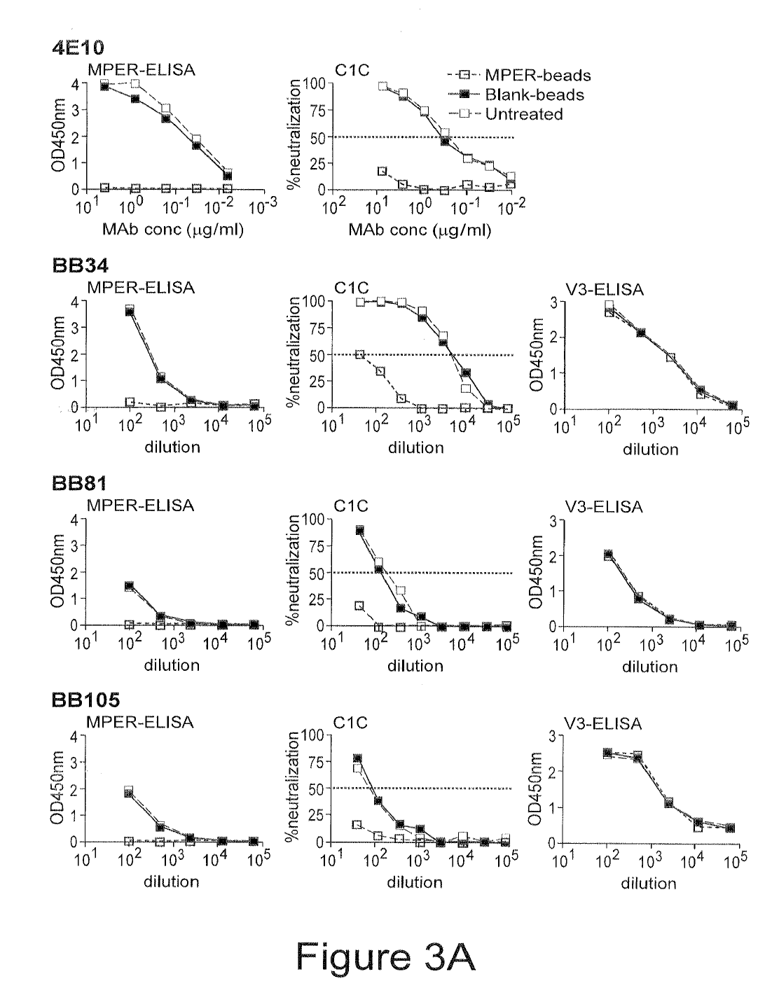

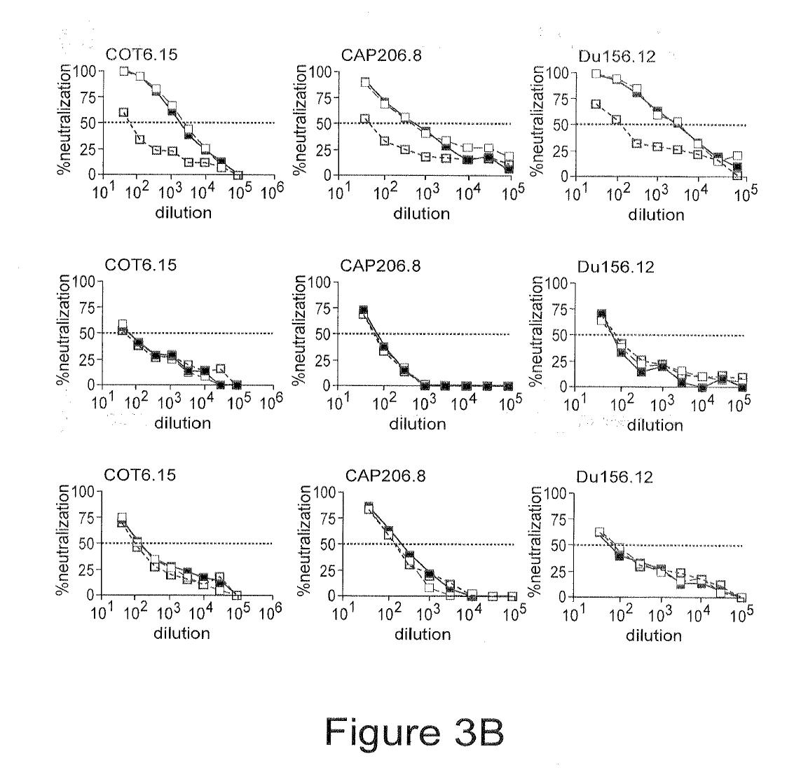

To examine the contribution of anti-MPER antibodies to heterologous neutralization, a method was devised to specifically adsorb these antibodies with magnetic beads coated with a peptide containing the MPER sequence. First tested were three plasma samples from the BB cohort, BB34, BB81, and BB105, which were previously found to have anti-MPER antibody titers of 1:4,527, 1:264, and 1:80, respectively (Gray et al, J. Virol. 83:8925-8937 (2009)). The monoclonal antibody (MAb) 4E10 was used as a positive control. The effective depletion of the anti-MPER antibodies was demonstrated by the loss of binding in an MPER-peptide ELISA, as well as a reduction in neutralization of the HIV-2-HIV-1 MPER chimeric virus C1C for all three plasmas and MAb 4E10 (FIG. 3A). There was no change in ELISA reactivity to a V3 peptide after treatment of samples with the blank or MPER-peptide-coated beads, demonstrating that the anti-MPER antibodies were specifically depleted from the plasma (FIG. 3A).

The adsorbed plasmas and their corresponding controls were tested for neutralization of three heterologous subtype C viruses, COT6.15 CAP206.8, and Du156,12. The depletion of anti-MPER antibodies affected the heterologous neutralizing activity of only plasma BB34. The other two plasmas retained their neutralizing activities despite the efficient removal of anti-MPER antibodies (FIG. 3B). This indicated that anti-MPER antibodies in BB81 and BB105 were not involved in the neutralization of these viruses. Since the anti-MPER titers of these two plasmas were substantially lower than that of BB34, this suggested that high anti-MPER titers may be required to mediate the neutralization of primary viruses. This notion is supported by the observation that the HIV-2-HIV-1 MPER chimeras were 1 to 2 log units more sensitive to the MAbs 4E10 and Z13e1 than HIV-1 primary viruses (Binley et al, J. Virol, 82:11651-11668 (2008)). The decision was, therefore, made to identify additional samples with high anti-MPER antibody titers for further experiments.

Screening for Broadly Cross-Neutralizing Plasma Samples Containing Anti-MPER Antibodies.

Three plasma samples with broadly cross-neutralizing activities and high titers of anti-MPER antibodies were identified following a comprehensive screening of three cohorts of chronically infected individuals (Table 2). BB34, described above, was one of 70 plasmas collected from HIV-infected blood donors, 16 of which were found to be broadly neutralizing (Gray et al, J. Virol 83:8925-8937 (2009)). Of these, 11 had anti-MPER antibodies; however, only BB34 had anti-C1C titers above 1:1,000. Also tested were plasmas from 18 participants in the CAPRISA cohort, corresponding to 3 years postinfection. Four of these were able to neutralize 50% or more of the subtype C primary viruses, two of which had anti-MPER antibodies. Of these, only CAP206 had titers above 1:1,000 and bound the linear peptide in an ELISA, Plasma SAC21 was selected from a second group of 68 blood donors (the SAC cohort), 4 of which had neutralization breadth and anti-MPER antibody titers above 1:1,000, However, only SAC21 bound the MPER peptide in an ELISA.

TABLE 2
Screening for broadly cross-neutralizing plasma samples
containing anti-MPER antibodies

| | Value in: | | |
|---|---|---|---|
| Parameter^{a} | BB cohort | CAPRISA | SAC cohort |
| Total no. of plasmas | 70 | 18 | 68 |
| No. (%) BCN | 16 (23)^{b} | 4 (22)^{c} | 17 (25)^{d} |
| No. of BCN anti-MPER | 11 | 2 | 6 |
| antibodies positive | | | |
| No. of BCN anti-MPER | 1 | 1 | 4 |
| titers >1:1,000 | | | |
| No. MPER peptide binding | 1 | 1 | 1 |
| Sample analyzed | BB34 | CAP206 | SAC21 |

^{a}BCN, broadly cross-neutralizing. Anti-MPER activity was defined as neutralization of the HIV-2-HIV-1 MPER chimeric virus C1C.
^{b}BCN plasmas were defined as able to neutralize at least 8 of 10 viruses tested (12).
^{c}BCN plasmas were defined as able to neutralize at least 8 of 12 viruses from the tier 2 subtype C virus panel.
^{d}BCN plasmas were defined as able to neutralize at least four of six viruses tested.

The levels of anti-MPER antibodies in these three plasma samples were high when tested against the HIV-2-HIV-1 MPER chimera C1C, with ID₅₀ titers of 1:4,802 for BB34,1:4,527 for CAP206, and 1:3,157 for SAC21. The extent of neutralization breadth of these plasmas was determined using a large panel of envelope-pseudotyped viruses of subtype A (n=5), B (n=13), C (n=24), and D (n=1). Plasma BB34 was able to neutralize 60% of all the viruses tested, while CAP206 neutralized 50% and SAC21 neutralized 47% of the panel.

Anti-MPER Antibodies Mediate Heterologous Neutralization.

To determine how much of the breadth in these three plasma samples was MPER mediated, this antibody specificity was deleted using peptide-coated beads and the adsorbed plasmas were tested against viruses that were neutralized at titers above 1:80. The percentage reduction in the ID50 after adsorption on MPER-peptide-coated beads relative to the blank beads was calculated for each virus. Reductions of more than 50% were considered significant. Neutralization of C1C was considerably diminished by the removal of anti-MPER in all three plasmas (Table 3). Similarly, there was a substantial decrease in the neutralization of the majority of primary viruses tested. For BB34, 77% (17/22) of the viruses tested with the adsorbed plasma showed evidence that neutralization was mediated by anti-MPER antibodies, while for CAP206 and SAC21, it was 68% (13/19) and 46% (6/13), respectively. None of the subtype A and D viruses were neutralized significantly (<50%) by the anti-MPER antibodies in these plasmas, although only a few clones were available to test. Neutralization of the subtype B viruses appeared to be as effective as subtype C virus neutralization. Overall, these results suggested that the anti-MPER antibodies found in these HIV-1 subtype C plasma samples were largely responsible for the observed heterologous neutralization.

TABLE 3
Effect of anti-MPER antibody adsorbtions on
neutralization breadth

| | | ID₅₀ | | % |
|---|---|---|---|---|
| Subtype | Virus | Blank^{a} | MPER^{b} | Reduction^{c} |
| Adsorbed BB34 | | | | |
| plasma | | | | |
| HIV-2/HIV-1 | C1C | 4,802 | 41 | 99 |
| MPER | | | | |
| Subtype C | COT6.15 | 1,350 | 65 | 95 |
| | CAP85 9 | 7,134 | 1,140 | 84 |
| | CAP88 B5 | 258 | <40 | 84 |
| | CAP206 8 | 1,350 | 86 | 94 |
| | CAP210 B8 | 148 | 102 | 31 |
| | CAP228 51 | 245 | 73 | 70 |
| | CAP255 16 | 164 | <40 | 76 |
| | Du151.2 | 484 | 636 | 0 |
| | Du422.1 | 155 | <40 | 74 |
| | Du156.12 | 3,869 | 151 | 96 |
| | ZM197M.PB7 | 1,068 | <40 | 96 |
| | ZM233M.PB6 | 219 | 66 | 70 |
| | ZM135M.PL10a | 1,651 | 250 | 85 |
| Subtype B | 6535.3 | 549 | 102 | 81 |
| | QHO692.42 | 179 | 42 | 77 |
| | CAAN5342.A2 | 139 | 129 | 7 |
| | TRO.11 | 646 | <40 | 94 |
| | SC422661.8 | 758 | 175 | 77 |
| | REJO4541.67 | 331 | 80 | 76 |
| | JR-FL | 129 | <40 | 69 |
| Subtype A | 92RW009 | 1,296 | 827 | 32 |
| Subtype D | 92UG024 | 1,480 | 1,006 | 32 |
| Adsorbed CAP206 | | | | |
| plasma | | | | |
| HIV-2/HIV-1 | C1C | 4,527 | 222 | 95 |
| MPER | | | | |
| Subtype C | COT6.15 | 1,236 | 109 | 91 |
| | CAP45 G3 | 4,720 | 193 | 96 |
| | CAP63 A9 | 180 | 132 | 27 |
| | CAP85 9 | 2,856 | 352 | 88 |
| | CAP88 B5 | 223 | <40 | 82 |
| | CAP206 8 | 1,870 | 1,555 | 17 |
| | Du151.2 | 105 | <40 | 62 |
| | Du422.1 | 165 | 47 | 72 |
| | Du156.12 | 692 | 57 | 92 |
| | Du172.17 | 234 | <40 | 83 |
| | ZM197M.PB7 | 309 | 82 | 73 |
| | ZM135M.PL10a | 248 | 91 | 63 |
| Subtype B | QHO692.42 | 383 | 66 | 83 |
| | AC10.0.29 | 111 | 47 | 58 |
| | WITO4160.33 | 144 | 99 | 31 |
| | TRO.11 | 491 | <40 | 92 |
| Subtype A | 92RW009 | 915 | 793 | 13 |
| | Q23.17 | 320 | 340 | 0 |
| Subtype D | 92UG024 | 1,556 | 1,268 | 19 |
| Adsorbed SAC21 | | | | |
| plasma | | | | |
| HIV-2/HIV-1 | C1C | 3,157 | 246 | 92 |
| MPER | | | | |
| Subtype C | COT6.15 | 183 | <40 | 78 |
| | CAP85 9 | 447 | 276 | 38 |
| | CAP88 B5 | 88 | 42 | 52 |
| | CAP206 8 | 361 | 140 | 61 |
| | CAP255 16 | 109 | 115 | 0 |
| | Du151.2 | 117 | 69 | 41 |
| | ZM197M.PB7 | 117 | 85 | 27 |
| | ZM233M.PB6 | 100 | 79 | 21 |
| | ZM135M.PL10a | 1,114 | 301 | 73 |
| Subtype B | TRO.11 | 147 | 47 | 68 |
| | SC422661.8 | 88 | <40 | 55 |
| Subtype A | 92RW009 | 1,665 | 1,045 | 37 |
| Subtype D | 92UG024 | 1,889 | 1,491 | 21 |

^{a}ID₅₀ of plasmas adsorbed on blank beads. These titers were similar to the ID₅₀ obtained with the untreated sera.
^{b}ID₅₀ of plasmas adsorbed on beads coated with the MPER peptide.
^{c}Percentage reduction in ID₅₀ due to adsorption on MPER-coated beads (1 − MPER/blank). Cases where the percent reduction was >50% are in boldface.

Potencies of Eluted anti-MPER Antibodies.

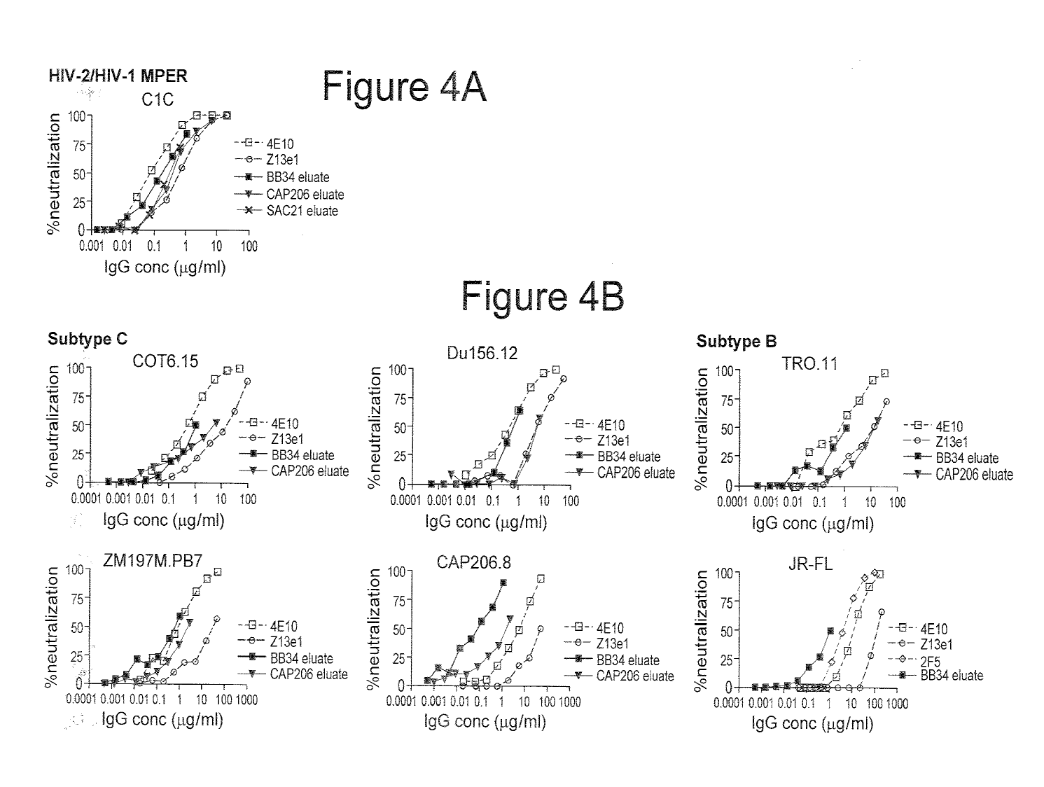

That the adsorbed antibodies had heterologous neutralizing activity was confirmed by assaying antibodies eluted from the MPER-peptidecoated beads. The eluates from all three plasmas neutralized C1C efficiently (FIG. 4A), BB34 was the most potent, with an IC50 of 0.18 μg/ml, while CAP206 and SAC21 were similar at 0.39 and 0.31 μg/ml, respectively. The eluates were also tested against four subtype C and one subtype B primary viruses that were sensitive to all three plasmas, and BB34 was also tested against JR-FL (FIG. 4B). The BB34 eluate was able to neutralize all six viruses with potency comparable to or greater than those of the MPER MAbs. Thus, the virus CAP206.8 was neutralized over 10-fold more efficiently by BB34 eluates than by MAb 4E10. For JR-FL, the BB34MPER eluate was even more effective than MAbs 2F5, 4E10, and Z13e1. The eluate from CAP206 was less potent and more comparable to the activity of MAb Z13e1. Interestingly, it was most potent against the CAP206.8 virus, suggesting a role for these anti-MPER antibodies in autologous neutralization. Despite multiple attempts, the antibody concentration of the SAC21 eluates was too low, and neutralization of viruses other than C1C was not observed. Similarly, the BB34 and CAP206 eluates did not have activity against viruses that the plasma neutralized at a low ID50, such as CAP88.B5 and Du151.2 (data not shown). Eluates from blank beads, used as negative controls, did not show activity against any of the viruses tested (data not shown).

IgG Subclasses in Plasma and Eluates.

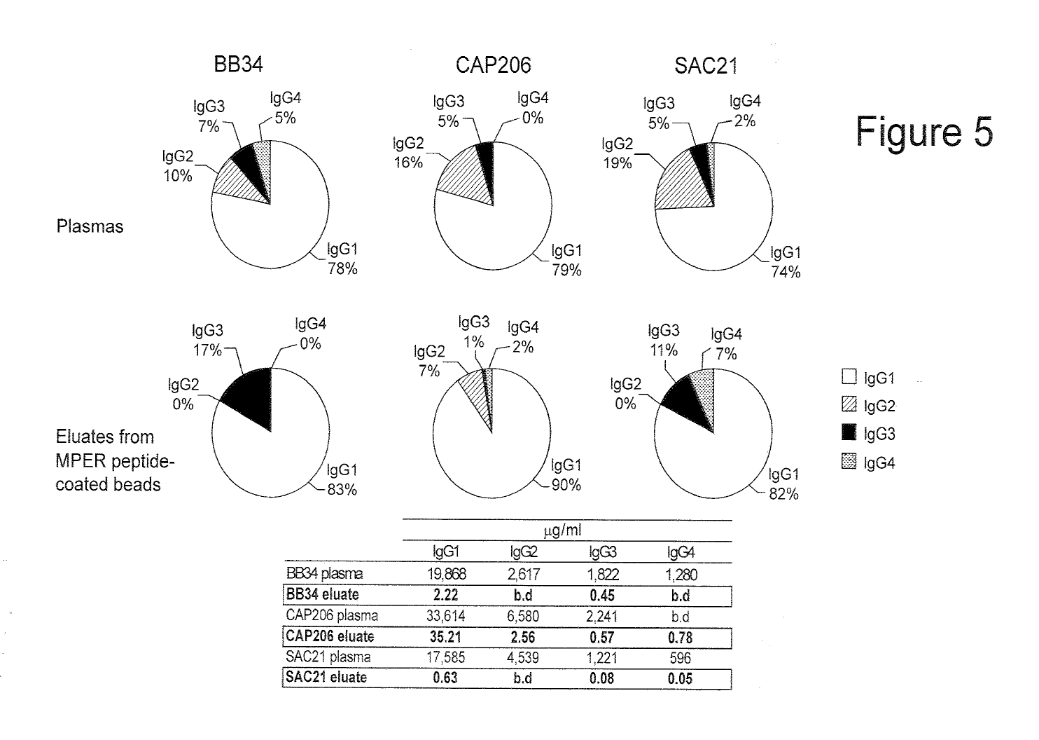

To establish the nature of these anti-MPER antibodies, the IgG subclass profiles of the antibodies eluted from the beads was determined and compared to those of the parent plasmas. All three plasma samples displayed the classical profile of IgG1>IgG2>IgG3>IgG4, although each had a different subclass distribution (FIG. 5). The eluates from the MPER beads were enriched in some subclasses. The BB34 eluate was enriched in IgG1 and IgG3 antibodies, while IgG2 and IgG4 were below detection. The CAP206 eluate was enriched in IgG1 and IgG4, while SAC21 was enriched in IgG1, IgG3, and IgG4 compared to whole plasma.

IgG3 Anti-MPER Antibodies Mediate Neutralization in Plasma BB34.

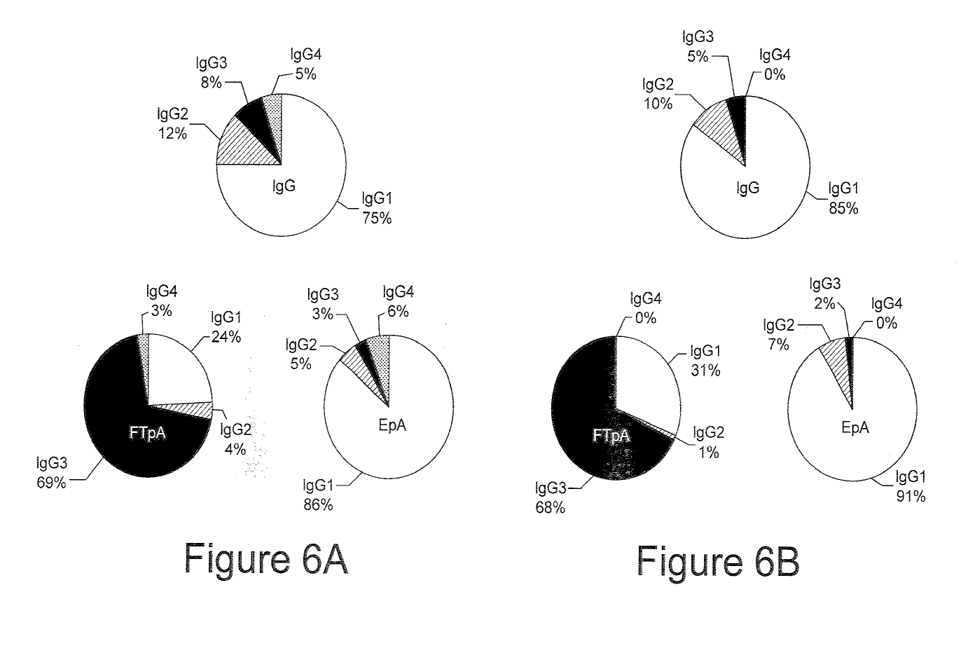

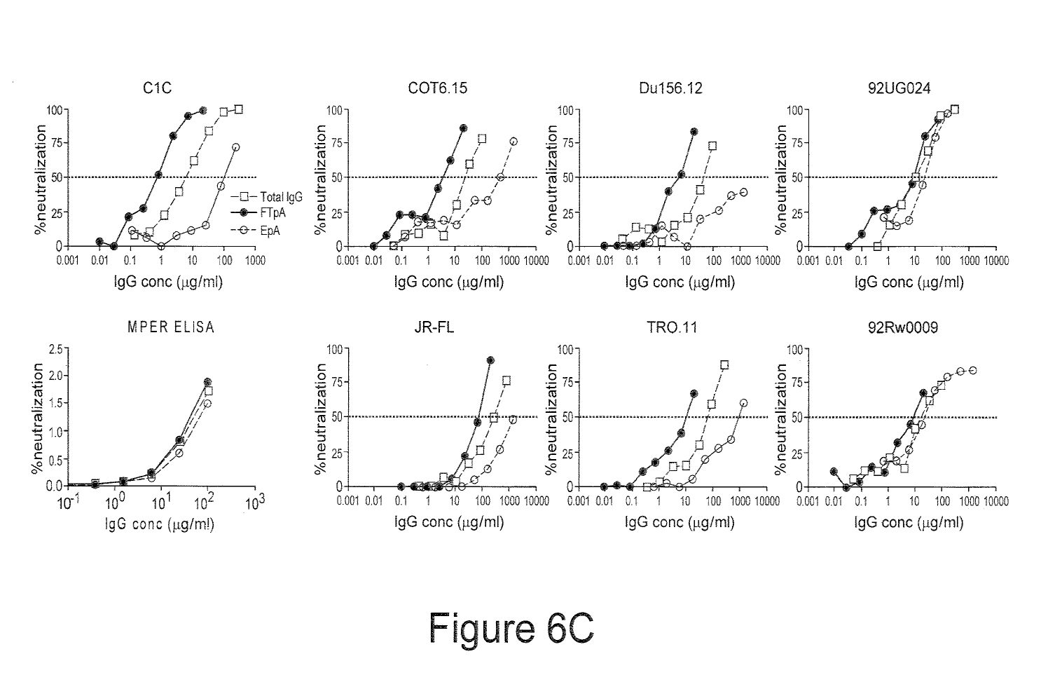

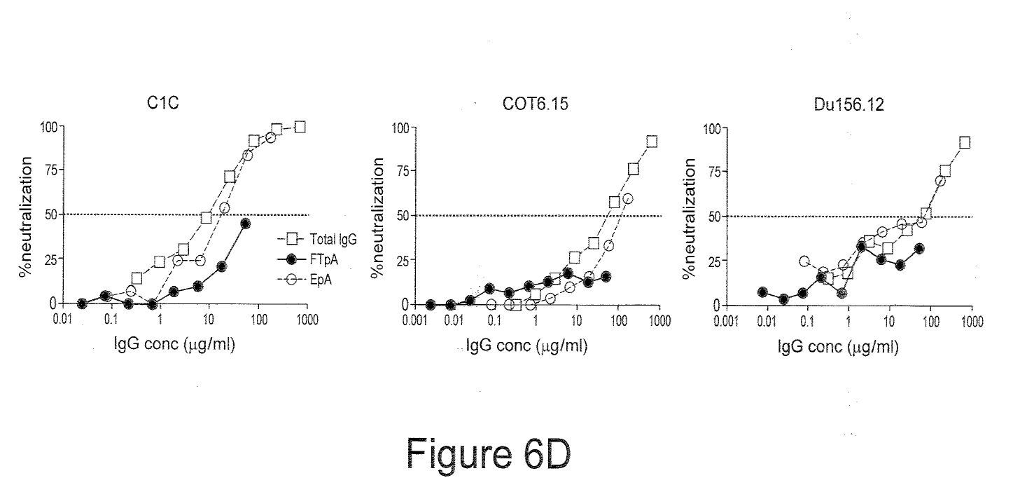

Given that the eluates from BB34 were enriched in IgG3 antibodies, the decision was made to explore the contribution of this IgG subclass to anti-MPER neutralization. Total IgG was extracted from the plasmas using a protein G column. This was followed by fractionation through a protein A column, which specifically excludes IgG3 antibodies. The fractions were tested for their IgG subclass profiles to corroborate that IgG3 antibodies were enriched in the protein A column flowthrough (FTpA) and excluded in the eluate (EpA) (FIG. 6A). Binding to the MPER peptide and the neutralizing activities of the fractions were compared after their total IgG concentrations were standardized. Interestingly, while no differences in binding were observed between the fractions, the FTpA fraction showed a 100-fold increase in neutralization of C1C compared to the EpA fraction (FIG. 6C). This suggested that most of the anti-MPER activity resided within the IgG3 fraction. Similar results were found in the neutralization of the viruses COT6.15, Du156.12, JR-FL, and TRO.11. However, for viruses 92Rw0009 and 92UG024, no differences in neutralization were noted between the FTpA and the EpA fractions. This corresponded to previous observations showing that these viruses were not neutralized via anti-MPER antibodies (Table 3).

To determine if IgG3-mediated neutralization was a general feature of cross-neutralizing anti-MPER antibodies, similar experiments were performed with the CAP206 plasma. The FTpA fraction of CAP206 was significantly enriched for IgG3 antibodies, similar to BB34 (FIG. 6B). However, the FTpA fraction had little to no neutralizing activity, while the EpA fraction clearly recapitulated the activity of the original IgG pool (FIG. 6D). This suggested that in CAP206, anti-MPER neutralizing antibodies were not IgG3.

MPER Epitope Mapping.

To characterize the epitopes recognized by these anti-MPER antibodies, they were tested against HIV-2/HIV-1 chimeras containing portions of the MPER (Binley et al, J. Virol. 82:11651-11668 (2008), Gray et al, J. Virol. 82:2367-2375 (2008), Gray et al, J. Virol. 81:6187-6196 (2007)). All three plasmas showed similar patterns of neutralization, mapping to an epitope in the C terminus of the MPER (Table 4). These anti-MPER antibodies were not identical to 4E10, as they failed to neutralize the C6 chimera, which contains the minimal residues for 4E10 neutralization. They were, however, dependent on a tryptophan at position 670 for recognition, as substantial differences in neutralization were observed between the chimeras C4 and C4GW. This is similar to the neutralization pattern seen with MAb Z13e1.

TABLE 4
Mapping of anti-MPER neutralizing antibodies

| | | Neutralization^{b} | | | Plasma ID₅₀ | | |
|---|---|---|---|---|---|---|---|
| Chimera | MPER sequence^{a} | 2F5 | 4E10 | Z13e1 | BB34 | CAP206 | SAC21 |
| 7312A | NMYEL₆₆₀QKLNSWDVFG₆₇₀NWFDLASWVK₆₈₀YIQYGVYIV | − | − | − | <20 | 21 | <20 |
| C1 | NMYEL₆₆₀LALDKWASLW₆₇₀NWFDITKWLW₆₈₀YIKYGVYIV | ++ | ++ | ++ | 5,560 | 3,903 | 3,871 |
| C1C | NMYEL₆₆₀LALDSWKNLW₆₇₀NWFDITKWLW₆₈₀YIKYGVYIV | − | ++ | ++ | 3,945 | 2,867 | 2,733 |

| | | | | | | | |
|---|---|---|---|---|---|---|---|
| C1C F/L | NMYEL₆₆₀LALDSWKNLW₆₇₀NWLDITKWLW₆₈₀YIKYGVYIV | − | − | + | 1,779 | 2,449 | 1,802 |
| C3 | NMYEL₆₆₀LALDKWASLW₆₇₀NWFDLASWVK₆₈₀YIQYGVYIV | ++ | − | − | <20 | <20 | <20 |
| C7(2F5) | NMYEL₆₆₀QALDKWAVFG₆₇₀NWFDLASWVK₆₈₀YIQYGVYIV | ++ | − | − | <20 | <20 | <20 |
| C6(4E10) | NMYEL₆₆₀QKLNSWDVFG₆₇₀NWFDITSWIK₆₈₀YIQYGVYIV | − | ++ | − | <20 | <20 | <20 |
| C4 | NMYEL₆₆₀QKLNSWDVFG₆₇₀NWFDITKWLW₆₈₀YIKYGVYIV | − | ++ | +/− | <20 | 723 | 189 |
| C4GW | NMYEL₆₆₀QKLNSWDVFW₆₇₀NWFDITKWLW₆₈₀YIKYGVYIV | − | ++ | ++ | 7,482 | 3,067 | 2,987 |
| C8 | NMYEL₆₆₀QKLNSWDSLW₆₇₀NWFDITKWLW₆₈₀YIKYGVYIV | − | ++ | + | 3,351 | 2,538 | 1,199 |

^{a}Grafted amino acids are indicated in italics, with the 7312A residues in lightface. Further mutations on the chimeras are in boldface.
^{b}Neutralization by MAbs 2F5, 4E10, and Z13e1 are qualitatively indicated relative to the titers obtained with the C1 chimera. −, no neutralization; ++, neutralization similar to that of C1; +, neutralization within 3-fold of that of C1; +/−, neutralization within 10-fold of that of C1.

To finely map these novel epitopes, alanine-scanned mutants were constructed from positions 662 to 680 of the MPER in the subtype C virus COT6.15 (Table 5). The alanine at position 662 was changed to a glycine residue. MAb Z13e1 did not effectively neutralize COT6.15, possibly due to a serine substitution in position 671 (Nelson et al, J. Virol. 81:4033-3043 (2007)), and therefore this MAb was not used in the characterization of these mutants. Many of the COT6.15 mutants showed increased sensitivity to neutralization by MAb 4E10 and the three plasmas (Table 5). Similar enhancement has been reported previously using mutants of the JR-2 strain (Nelson et al, J. Virol. 81:4033-3043 (2007), Zwick et al, J. Virol. 75:10892-10905 (2001)), which may be related to distortion of the MPER structure, resulting in increased antigenic exposure. However, major changes were not observed in the infectivities of the mutant viruses. Neutralization by 4E10 was ablated by previously defined residues with changes at W672, F673, T676, and W680, substantially reducing sensitivity to the MAb (Zwick et al, J. Virol. 75:10892-10905 (2001)). The three plasma samples effectively neutralized most alanine mutants (Table 5). The mutation W670A affected neutralization by BB34 and to a lesser extent by SAC21, supporting the above findings with the HIV-2 chimeras. However, this mutation did not affect CAP206 neutralization. This is consistent with the observation that CAP206 had the least disparity in titers between the C4 and C4GW chimeras (Table 4). Nonetheless, the decreased sensitivity of C4 to CAP206 may suggest that the residue is more critical for the correct presentation of this epitope in the context of the HIV-2 envelope. The F673A mutation eliminated recognition by SAC21 with no effect on BB34 and CAP206 neutralization. The mutation D674A abrogated neutralization by all three plasmas. As this residue is highly polymorphic among HIV-1 strains, D674 was further mutated to serine or asparagine, the other two common amino acids found at this position. D674N had little effect on neutralization, with only a twofold drop in the ID50, while the D6745 mutation affected recognition by all three plasmas. In summary, these plasmas recognized overlapping but distinct epitopes within the C-terminal region of the MPER that did not correspond to the previously defined 4E10 or Z13e1 epitope.

TABLE 5
Relative neutralization of pseudotyped COT6.15 envelope MPER mutants^{a}

| | 4E10 | | BB34 | | CAP206 | | SAC21 | |
|---|---|---|---|---|---|---|---|---|
| COT6.15 | IC₅₀ | Ratio^{b} | ID₅₀ | Ratio^{c} | ID₅₀ | Ratio^{c} | ID₅₀ | Ratio^{c} |
| Wild type | 0.9 | 1.0 | 1,392 | 1.0 | 1,256 | 1.0 | 317 | 1.0 |
| A662G | 0.12 | 0.1 | 4,899 | 0.3 | 2,443 | 0.5 | 978 | 0.3 |
| L663A | 0.02 | 0.0 | 8,714 | 0.2 | 7,971 | 0.2 | 5,660 | 0.1 |
| D664A | 0.77 | 0.9 | 1,149 | 1.2 | 844 | 1.5 | 238 | 1.3 |
| S665A | 0.14 | 0.2 | 5,495 | 0.3 | 1,562 | 0.8 | 1,787 | 0.2 |
| W666A | 0.51 | 0.6 | 5,554 | 0.3 | 4,294 | 0.3 | 446 | 0.7 |
| K667A | 0.05 | 0.1 | 3,261 | 0.4 | 1,694 | 0.7 | 1,734 | 0.2 |
| N668A | 1.3 | 1.4 | 831 | 1.7 | 425 | 3.0 | 208 | 1.5 |
| L669A | 0.05 | 0.1 | 3,847 | 0.4 | 3,138 | 0.4 | 1,195 | 0.3 |
| W670A | 0.11 | 0.1 | 132 | 10.5 | 1,054 | 1.2 | 105 | 3.0 |
| S671A | 0.04 | 0.0 | 3,102 | 0.4 | 1,614 | 0.8 | 928 | 0.3 |
| W672A | >25 | >25 | 2,959 | 0.5 | 2,244 | 0.6 | 468 | 0.7 |
| F673A | >25 | >25 | 779 | 1.8 | 498 | 2.5 | <50 | >6.3 |
| D674A | 1.4 | 1.6 | <50 | >25 | <50 | >25 | <50 | >6.3 |
| D674S | 2.49 | 2.8 | <50 | >25 | 90 | 14.0 | <50 | >6.3 |
| D674N | 0.33 | 0.4 | 663 | 2.1 | 643 | 2.0 | 149 | 2.1 |
| I675A | 0.04 | 0.0 | 4,069 | 0.3 | 2,065 | 0.6 | 718 | 0.4 |
| T676A | 21.77 | 24.2 | 2,380 | 0.6 | 895 | 1.4 | 524 | 0.6 |
| K677A | 0.05 | 0.1 | 4,671 | 0.3 | 2,151 | 0.6 | 1154 | 0.3 |
| W678A | 0.05 | 0.1 | 3,842 | 0.4 | 1,885 | 0.7 | 1,007 | 0.3 |
| L679A | 0.09 | 0.1 | 2,085 | 0.7 | 1,448 | 0.9 | 225 | 1.4 |
| W680A | 10.89 | 12.1 | 731 | 1.9 | 904 | 1.4 | 142 | 2.2 |

^{a}Cases with more than a 3-fold drop in the ID₅₀ or IC₅₀ are in boldface.
^{b}(Mutant IC₅₀)/(wild-type IC₅₀) ratio.
^{c}(Wild-type ID₅₀)/(mutant ID₅₀) ratio.

In this study, it has been clearly demonstrated that anti-MPER antibodies in three broadly cross-neutralizing plasmas were largely responsible for the heterologous neutralization displayed by these samples. For most viruses, the bulk of the neutralizing activity could be attributed to this single antibody specificity. Furthermore, the data suggested that these antibodies were as potent as existing MAbs and defined novel epitopes within the MPER. These data reinforce the potential of the HIV-1 gp41MPER as a neutralizing-antibody vaccine target.

A significant association was previously shown between the presence of anti-MPER antibodies and neutralization breadth in plasma samples from a cohort of chronically infected blood donors (Gray et al, J. Virol. 83:8925-8937 (2009)). At least in some cases, anti-MPER antibodies are primarily responsible for this neutralizing activity. The levels of breadth displayed by these three HIV-1 subtype C plasma samples varied, with BB34 being the broadest and CAP206 and SAC21 neutralizing about half the viruses tested. Of those viruses neutralized by BB34 and CAP206, approximately 70% were neutralized via anti-MPER antibodies, and in the majority of cases, these antibodies mediated almost all the activity. The anti-MPER antibodies in SAC21 neutralized fewer viruses, and often they only partially contributed to the overall neutralization, probably due to smaller amounts of specific IgG in the sample. For all three plasmas, there were examples where the adsorption of anti-MPER antibodies did not remove all the neutralizing activity or in some cases had no effect. The latter suggests that other specificities distinct from the adsorbed anti-MPER antibodies were also present in these plasmas. The residual neutralization of C1C by depleted CAP206 and SAC21 plasmas suggested that in some cases they may also be MPER antibodies that failed to bind the linear peptide. This is in line with the observations by others that more than one specificity may be involved in the neutralization breadth displayed by plasmas from some HIV-1-infected individuals (Binley et al, J. Virol. 82:11651-11668 (2008), Doria-Rose et al, J. Virol. 83:188-199 (2009), Li et al, J. Virol. 83:1045-1059 (2009), Sather et al, J. Virol. 83:757-769 (2009)).

Testing of the antibodies eluted from the MPER peptide made it possible to conclusively show that these antibodies mediated cross-neutralization. The potency of the eluted antibodies recapitulated the activity in the original plasma samples, although the IC50 and ID50 values did not always correlate. This may be due to other non-MPER neutralizing antibodies present in these samples, as described above, or perhaps loss of activity during the elution process. Eluates are likely to contain mixtures of MPER-specific antibodies that may differ in binding affinity, as well as neutralization capacity, and thus represent considerably more of a technical challenge than testing purified MAbs. Even if the elution data are more qualitative than quantitative, they nevertheless show that the potencies of these antibodies are in the range of the current MAbs. Interestingly, the CAP206 eluate efficiently neutralized the autologous virus, despite the fact that no significant reduction in the 1050 was observed after depletion of anti-MPER antibodies from the plasma sample (Table 3). It is possible that other autologous neutralizing-antibody specificities overshadowed the activities of the anti-MPER antibodies in this plasma sample.

The neutralizing anti-MPER antibodies in plasma BB34 were found to be mainly IgG3. It is interesting that the original hybridoma-derived broadly neutralizing anti-MPER MAbs 4E10 and 2F5 were of the IgG3 subclass (Kurnert et al, Biotechnol. Bioeng. 67:97-103 (2000)) and the neutralizing fraction of a polyclonal human HIV immune globulin was also reported to be IgG3 (Scharf et al, J. Virol. 75:6558-6565 (2001)). IgG3s have a highly flexible hinge region that has been proposed to facilitate access to the MPER and that is thought to be partly buried in the viral membrane and enclosed by the gp120 protomers. However, for both MAbs, a change to IgG1 did not affect the neutralization capacity, suggesting that IgG3s are not essential for MPER-mediated neutralization (Kurnert et al, Biotechnol. Bioeng. 67:97-103 (2000), Kunert et al, Hum. Retrovir. 20:755-762 (2004)). Indeed, for CAP206, the IgG3-enriched fraction had less activity, and in this case, neutralization was due to either IgG1 or IgG2. While there was an enrichment of IgG3 in SAC21 eluates, the low potency of these antibodies precluded them from being tested further. Both BB34 and SAC21 were from blood donors with an unknown duration of infection, while CAP206 has been followed prospectively for 3 years since seroconversion. Although IgG3 has been reported to appear early in infection, the anti-MPER response will be monitored in CAP206 to see if the IgG subclass profile, antibody specificities, or neutralization titers change over time.

The binding of all three anti-MPER plasma antibodies depended on the residue at position 674 in the MPER, which has been shown to be the most critical for Z13e1 recognition (Pejchal et al, J. Virol. 83:8451-8462 (2009)). The immunogenicity of this residue may be related to its location in the hinge region of the MPER (Pejchal et al, J. Virol. 83:8451-8462 (2009), Song et al, Proc. Natl. Acad. Sci. USA 106:9057-9062 (2009), Sun et al, Immunity 28:52-63 (2008)). However, the high level of polymorphism at this position is considered to be one of the main reasons why the Z13 e1 MAb neutralizes a narrower set of viruses than the 4E10 MAb. In contrast to MAb 2F5, which seldom neutralizes subtype C viruses due to a subtype-associated polymorphism at position 665 (Binley et al, J. Virol. 82:11651-11668 (2008), Gray et al, PLoS Med. 3:e255 (2006)), the residue at position 674 is not associated with a particular subtype. This is consistent with the finding that subtype B and C viruses were equally neutralized by MPER antibodies present in all three plasmas. In addition to this common residue, BB34 and SAC21 also depended on W670, which is not implicated in either 4E10 or Z13e1 recognition. SAC21 showed some overlap with the 4E10 MAb, since it was affected by the F673A mutation. However, the identities of the precise residues required by these antibodies indicated that they are distinct from 4E10 and Z13e1. Furthermore, analysis of the MPER sequences of the viruses neutralized by these plasmas suggested that the residue at position 674 affects their sensitivity, with the majority of viruses harboring a serine showing resistance. However, not all viruses with an aspartic or asparagine residue at position 674 and, even more, with the same MPER sequence were neutralized equally, suggesting that features outside this region may modulate the presentation of this epitope, as suggested by previous studies (Binley et al, J. Virol. 82:11651-11668 (2008), Gray et al, J. Virol. 82:2367-2375 (2008)).

The presence of anti-MPER antibodies in broadly cross-neutralizing subtype B plasmas has been reported recently by others. Li and colleagues found that neutralization of the JR-FL virus by plasma no. 20 was out-competed by a peptide covering the 4E10 epitope, although the extent of the contribution of this specificity to breadth was not determined (Li et al, J. Virol. 83:1045-1059 (2009)). Sather and coworkers found 4E10-like activity in plasma VC10008 (Sather et al, J. Virol. 83:757-769 (2009)); however, this sample did not neutralize some 4E10-sensitive viruses, suggesting differences in their specificities. Neither of these studies investigated the precise epitopes recognized by these potentially novel antibodies, so it is not possible to determine if they differ from the ones identified here. A third study described an individual who developed antibodies that recognized a region overlapping the 2F5 epitope (Shen et al, J. Virol. 83:3617-3625 (2009)). Anti-MPER affinity-purified antibodies from this individual, SC44, displayed broad neutralizing activity. Similar to the study described above, which identified three samples from among 156 chronically infected individuals, the 2F5-like antibody found by Shen and colleagues was 1 of 311 plasmas analyzed (Shen et al, J. Virol, 83:3617-3625 (2009)).

The scarcity of these samples supports the notion that broadly neutralizing anti-MPER antibodies are seldom developed by HIV-1-infected individuals. Haynes et al. proposed that such antibodies are autoreactive and therefore eliminated through B-cell tolerance mechanisms (Haynes et al, Science 308:1906-1908 (2005)₃). While CAP206 did not have detectable levels of autoreactive antibodies, BB34 was positive for anti-double-stranded DNA antibodies and rheumatoid factor (Gray et al, J. Virol 83:8925-8937 (2009)). Another explanation for the paucity of such antibodies may be the short exposure time of this epitope during the formation of the fusion intermediate (Frey et al, Proc. Natl. Acad. Sci, USA 105:3739-3744 (2008)). Consistent with this, MAbs 2F5, 4E10, and Z13e1, as well as plasma BB34, neutralize JR-FL after CD4 and CCR5 attachment, when this occluded epitope is exposed (Binley et al, J. Virol, 77:5678-5684 (2003), Binley et al, J. Viral. 82:11651-11668 (2008)). Furthermore, BB34 neutralization was potentiated by coexpression of FcγRI on JC53b1-13 cells, also a feature of 2F5 and 4E10, possibly by providing a kinetic advantage through prepositioning of these antibodies close to the MPER (Perez et al, J. Virol. 83:7397-7410 (2009)). However, it remains unclear how these antibodies are induced in the context of natural infection despite the exposure constraints of this epitope. Perhaps these antibodies are elicited by more open conformations of the envelope glycoprotein that expose the MPER. Analysis of the autologous viruses that induce such responses may help to answer these questions.

It is noteworthy that the three cross-neutralizing antibodies identified here, while sharing some common residues, had distinct fine specificities. This suggests that the MPER can be recognized in a variety of conformations by the human immune system. It is therefore critical to isolate MAbs that define these novel epitopes within the MPER in order to facilitate a better understanding of the immunogenic structure of this region of gp41 and to identify new targets for HIV vaccine design.

### Example 2

Tetramers were prepared as described in U.S. application Ser. No. 12/320,709, filed Feb. 2, 2009, using the biotinylated MPR.03 peptide (sequence below and in FIG. 2A) with both allophycocyanin (APC) and in PacificBlue labeled streptavidins. They were titered on antibody-coated beads and on antibody expressing cell lines.

Biotinylated MPR.03 peptide
biotin-KKKNEQELLELDKWASLWNWFDITNWLWYIRKKK

Additionally, non-fluorochrome-labeled (“cold”) tetramers were prepared by using unlabeled streptavidin. This material was used for assays to characterize the antibodies produced.

Excess biotinylated peptide (approximately 8:1 molar ratio of peptide to streptavidin for cold tetramers and 33:1 molar ratio of peptide to streptavidin for fluorochrome-labeled tetramers) was incubated at 4° C. overnight and was isolated using gel filtration on Micro BioSpin 30 columns (BioRad Laboratories, Hercules, Calif.) or by concentration and washing using a Centriprep 30,000Da MWCO concentrator (Millipore, Billerica, Mass.). Peptides were checked for final concentration and tested on antibody-coated beads for specificity of binding. Final titers were determined using a combination of antibody-coated beads and antibody-expressing cell lines. Cold tetramers were confirmed to have activity by performing competition experiments with fluorochrome-labeled tetramers.

Using tetramers prepared as above, sorting experiments were performed using equimolar amounts of the tetramers in combination with a panel of monoclonal antibodies that can be used to identify B cells (Levesque et al, PLoS Med 6:e1000107 (2009)) on peripheral blood mononuclear cells from patient CAP206 and isolated as single cells into wells of a 96-well plate those cells that were labeled by both tetramers (FIG. 2C).

High-throughput isolation of immunoglobulin genes from single human B cells and expression as monoclonal antibodies can be carried out described by Liao et al, J. Virol. Methods 158:171-179 (2009).

Defining human B cell repertoires to viral pathogens is critical for design of vaccines that induce broadly protective antibodies to infections such as HIV-1 and influenza. Single B cell sorting and cloning of immunoglobulin (Ig) heavy- and light-chain variable regions (V_{H} and V_{L}) is a powerful technology for defining anti-viral B cell repertoires. However, the Ig-cloning step is time-consuming and prevents high-throughput analysis of the B cell repertoire. Novel linear Ig heavy- and light-chain gene expression cassettes were designed to express Ig V_{H} and V₁, genes isolated from sorted single B cells as IgG1 antibody without a cloning step. The cassettes contain all essential elements for transcriptional and translational regulation, including CMV promoter, Ig leader sequences, constant region of IgG1 heavy- or Ig light-chain, poly(A) tail and substitutable V_{H} or V_{L} genes. The utility of these Ig gene expression cassettes was established using synthetic V_{H} or V_{L} genes from an anti-HIV-1 gp41 mAb 2F5 as a model system, and validated further using V_{H} and V_{L} genes isolated from cloned EBV-transformed antibody-producing cell lines. Finally, this strategy was successfully used for rapid production of recombinant influenza mAbs from sorted single human plasmablasts after influenza vaccination. These Ig gene expression cassettes constitute a highly efficient strategy for rapid expression of Ig genes for high-throughput screening and analysis without cloning.

Immunoglobulin (Ig) is comprised of two identical heavy- and two identical light-chains. Ig heavy- and light-chain genes are produced by rearrangement of germline variable (V) and joining (J) gene segments at the light-chain locus, and by rearrangement of V, diversity (D) and J gene segments at the heavy-chain locus, respectively (Tonegawa, 1983; Diaz and Casali, 2002; Di Noia and Neuberger, 2007). Ig diversity is enhanced by somatic hypermutation of the rearranged genes (Kim et al., 1981; Di Noia and Neuberger, 2007). Antibody diversity allows the immune system to recognize a wide array of antigens (Honjo and Habu, 1985; Market and Papavasiliou, 2003). Antibodies represent the correlates of protective immunity to infectious agents (Barreto et al., 2006). Monoclonal antibodies (mAbs) are important tools for studying pathogenesis, the protein structure of infectious agents and the correlates of protective immunity, and are essential to the development of passive immunotherapy and diagnostics against infectious agents. Defining the molecular aspects of human B cell repertoires to viral pathogens is critical for designing vaccines to induce broadly protective antibody responses to infections such as HIV-1 and influenza. The traditional methods used for generating human mAbs include screening Epstein-Barr virus (EBV)-transformed human B cell clones or antibody phage display libraries. These methods are often time-consuming and can have low yields of pathogen-specific mAbs. Although electroporation (Yu et al., 2008) and use of B cell activation by oCPGs (Traggiai et al., 2004) have improved the efficiency for development of EBV-transformed antibody-secretion B cell lines, techniques for the isolation, sequencing and cloning of rearranged heavy- and light-chain genes directly from human B cells are of interest because they provide a means to produce higher numbers of specific human mAbs. It has been shown that rearranged Ig heavy- and light-chain variable regions (V_{II} and V_{L}) can be amplified from single B cells using RT-PCR (Tiller et al., 2008; Volkheimer et al., 2007; Wrammert et al., 2008), thus making it possible to produce mAbs recombinantly (Wardemann et al., 2003; Koelsch et al., 2007; Tiller et al., 2008; Wrammert et al., 2008). Generally, the expression of rearranged Ig genes as antibodies requires cloning of the amplified Ig V_{H} and V_{L} into eukaryotic cell to expression plasmids containing a transcription regulation control element such as the CMV promoter (Boshart et al., 1985), sequences encoding the Ig leader, heavy- and light-chain Ig constant regions and a poly(A) signal sequence (McLean et al., 2000; Connelly and Manley, 1988; Norderhaug et al., 1997). Thus, what is needed to profile the Ig repertoire following immunization or an infection is the ability to amplify large numbers of Ig genes using a strategy that circumvents the Ig cloning step and yields sufficient quantities of transiently expressed Ig to allow functional characterization of expressed Igs. Linear expression constructs generated by one-step PCR have been used for expression of vaccinia DNA topoisomerase I (Xiao, 2007) and HIV-1 envelope proteins (Kirchherr J L, 2007). To facilitate high throughput testing of amplified Ig V_{H} and V_{L} genes for antibody expression and specificity analysis, a strategy was designed that uses PCR and novel linear Ig heavy- and light-chain gene expression cassettes for rapid expression of Ig V_{H} and V_{L} genes as recombinant antibodies without cloning procedures.

### Materials and Methods

Antibodies, Cell Lines and Ig Heavy- and Light-Chain Genes

Anti-HIV-1 membrane proximal gp41 mAb 2F5 was purchased from Polymun Scientific (Vienna, Austria). DNA sequences encoding the variable region of 2F5 heavy- and light-chain (Ofek et al., 2004) were reconstructed using the amino acid sequences from PDB (PDBID:1TJG:H and 1TJG:L) and the published DNA sequence (Kunert et al., 1998). Sequences of a full-length IgG1 heavy gene (Strausberg et al., 2002) and a full-length kappa chain gene (Strausberg et al., 2002) that were modified to contain sequences encoding for the V_{H} and V_{L} of mAb 2F5 were de novo synthesized (Blue Heron, Bothell, Wash.).

The synthetic full-length 2F5 heavy- and light-chain genes were separately cloned into pcDNA3.1⁺ plasmid/hygro (Invitrogen, Carlbad, Calif.) that contains hygromycin resistant gene to facilitate screening of stably transfected-mammalian cell clones and resulted in plasmids HV13221 and HV13501, respectively. The HV13221 and HV13501 plasmids were used as sources of V_{H} and V_{L} chain sequences for method development and also used to generate stably transfected-293T cell line for producing purified recombinant 2F5 antibody, termed r2F5 HV01 mAB, as positive controls. A human embryonic kidney cell line, 293T, was obtained from the ATCC (Manassas, Va.), cultured in DMEM supplemented with 10% FCS and used for DNA transfections. A stably transfected-293T cell line was generated by co-transfection with plasmids HV13221 and HV13501 using PolyFect (Qiagen, Valencia, Calif.), grown in DMEM supplemented with 10% FCS and maintained in DMEM supplemented with 2% FCS for production of r2F5 HV01 mAb. Recombinant 2F5 HV01 mAb was purified from culture supernatants of the stably transfected-293T cells by anti-human Ig heavy chain specific antibody-agarose beads (Sigma, St. Louis, Mo.). A human B cell line, 08, that secretes antibody recognizing the HIV-1 gp41 immunodominant epitope, was generated by EBV-transformation of B cells in terminal ileum biopsy obtained from an acute/early HIV-1 positive subject (Hwang, unpublished). An EBV-transformed human B cell line, 7B2, that produces an anti-HIV-1 gp41 antibody (Binley et al., 2000) was kindly provided by James Robinson. G8 and 7B2 cell lines were grown in Hybridoma-SFM (Invitrogen, Carlsbad, Calif.). mAbs were purified from culture supernatants using a ProPur Protein G column (NuNC, Rochester, N.Y.).

Flow Cytometry and Cell Sorting

Blood samples were collected as part of an IRB-approved protocol from a volunteer who received Fluzone® 2007-2008 vaccination. Peripheral blood mononuclear cells (PBMC) were isolated from blood that was collected on day 0, 7 and 21. PBMC were suspended in RPMI culture medium containing 20% FCS and 7.5% DMSO and stored in vapor phase liquid nitrogen until use. Antibodies used for flow cytometry were anti-human IgG-PE, CD3 PE-Cy5, CD16 PE-Cy5, CD19 APC-Cy7, CD20 PE-Cy7, CD27 Pacific Blue, CD235a PE-Cy5, IgD PE, IgM FITC (BD Biosciences, San Jose, Calif.), CD14 PE-Cy5 and CD38 APC-Cy5.5 (Invitrogen, Carlsbad, Calif.). All antibodies were titered in advance and used at optimal concentrations for flow cytometry. Plasma cells gated as CD3−, CD14−, CD16−, CD235a−, CD19+, CD20low-neg, CD27hi, and CD38hi were sorted as single cells into 96-well PCR plates containing 20 μl/well of RT reaction buffer that included 5 μl of 5×First strand cDNA buffer, 0.5 μl of RNAseOut (Invitrogen, Carlsbad, Calif.), 1.25 μl of DTT, 0.0625 μl of Igepal and 13.25 μl of dH2O (Invitrogen, Carlsbad, Calif.). The plates were stored at −80° C. until use. Flow cytometric analysis and cell sorting were performed on a BD FACSAria (BD Biosciences, San Jose, Calif.) and the data were analyzed using FlowJo (Tree Star, Ashland, Oreg.).

Isolation of Ig Variable Region Transcripts from EBV-Transformed B Cells and Sorted Single Plasmablasts by RT-PCR

The genes encoding Ig V_{H} and V_{L} chains were amplified by RT and nested PCR using a modification of a previously reported method (Tiller et al., 2008). Briefly, synthesis of Ig V_{H} and V_{L} was performed in 96-well PCR plates containing cloned EBV-transformed B cells or sorted single human plasmablasts. The RT reaction was carried out at 37° C. for 1 hour after addition of 50 units/reaction Superscript III reverse transcriptase (Invitrogen, Carlsbad, Calif.) and 0.5 μM human IgG, IgM, IgD and IgA1, IgA2, Igκ and Igλ constant region primers (Table 6). After cDNA synthesis, V_{H}, V_{κ} and V_{λ} genes were amplified separately by two rounds of PCR in 96-well PCR plates in 50 μL reaction mixtures. The first-round of PCR contained 5 μL of RT reaction products, 5 units of HotStar Taq Plus (Invitrogen; Carlsbad, Calif.), 0.2 mM dNTPs, and 0.5 μM of either IgM, IgG, IgD, IgA1 and IgA2, or Igκ or Igλ constant region primers and is sets of IgH, Igκ or Igλ variable region primers (Tables 7 and 8). The first round of PCR was performed at 95° C.×5 min followed by 35 cycles of 95° C.×30 s, 55° C. (V_{H} and V_{κ}) or 50° C. (V_{λ}) δ 60 s, 72° C.×90 s, and one cycle at 72° C.×7 min. Nested second round PCR was performed with 2.5 μL of first-round PCR product, 5 units of HotStar Taq Plus, 0.2 mM dNTPs, 0.5 μM of either IgM, IgG, IgD, IgA1 and IgA2, or Igκ and Igλ nested constant region primers and sets of IgH, IgK or Ig2, nested variable region primers (Tables 9-11). During the second round of nested PCR, the IgH, Igκ and Igλ variable region primers were amplified in separate reaction mixes for each variable region primer. The second-round of PCR was performed at 95° C.×5 min followed by 35 cycles of 95° C.×30 s, 58° C. (V_{H}), 60° C. (V_{κ}) or 64° C. (V_{λ})×60 s, 72° C.×90 s, and one cycle at 72° C.×7 min. Samples of V_{H}, V_{κ} and V_{λ} chain PCR products were analyzed on 1.2% agarose gels. Bone marrow RNA (Clontech, Mountain View, Calif.) samples were included during all RT-PCR runs as positive controls. All primers used for the 2^{nd} round of PCR included tag sequences at the 5′ end of each primer (Tables 9-11). This permits assembly of the V_{H} and V_{L} genes into functional linear Ig gene expression cassettes as described below. All PCR products were purified using a Qiagen (Valencia, Calif.) PCR purification kit and sequenced in forward and reverse directions using an ABI 3700 instrument and BigDye® sequencing kit (Applied Biosystems, Foster City, Calif.). Sequences were analyzed using the IMGT information system (http://imgt.cines.fr/) to identify variable region gene segments and somatic mutations,

TABLE 6
Primers used for reverse transcriptase reaction.

| RT primer | 5′-3′ sequence |
|---|---|
| IgM-RT | ATG GAG TCG GGA AGG AAG TC |
| IgD-RT | TCA CGG ACG TTG GGT GGT A |
| IgE-RT | TCA CGG AGG TGG CAT TGG A |
| IgA1-RT | CAG GCG ATG ACC ACG TTC C |
| IgA2-RT | CAT GCG ACG ACC ACG TTC C |
| IgG-RT | AGG TGT GCA CGC CGC TGG TC |
| Cκ-new RT | GCA GGC ACA CAA CAG AGG CA |
| Cλ-new-ext | AGG CCA CTG TCA CAG CT |

TABLE 7
Primer pairs used for heavy chain and
kappa chain in first round PCR.

| | 5′-3′ sequence |
|---|---|
| forward | |
| primer | |
| V_{H}1 -Ext | CCA TGG ACT GGA CCT GGA GG |
| V_{H}2-Ext | ATG GAC ATA CTT TGT TCC A |
| V_{H}3-Ext | CCA TGG AGT TTG GGC TGA GC |
| V_{H}4-Ext | ATG AAA CAC CTG TGG TTC TT |
| V_{H}5-Ext | ATG GGG TCA ACC GCC ATC CT |
| V_{H}6-Ext | ATG TCT GTC TCC TTC CTC AT |
| V_{κ}1/2-Ext | GCT CAG CTC CTG GGG CT |
| V_{κ}3-Ext | GGA ARC CCC AGC DCA GC |
| V_{κ}4/5-Ext | CTS TTS CTY TGG ATC TCT G |
| V_{κ}6/7-Ext | CTS CTG CTC TGG GYT CC |
| reverse | |
| primer | |
| IgA-ext | CGA YGA CCA CGT TCC CAT CT |
| IgD-ext | CTG TTA TCC TTT GGG TGT CTG CAC |
| IgG-ext | CGC CTG AGT TCC ACG ACA CC |
| IgM-ext | CCG ACG GGG AAT TCT CAC AG |
| Cκ-ext | GAG GCA GTT CCA GAT TTC AA |

TABLE 8
Primer pairs used for lambda chain in first
round PCR.

| | 5′-3′ sequence |
|---|---|
| forward | |
| primer | |
| V_{λ}1-Ext | CCT GGG CCC AGT CTG TG |
| V_{λ}2-Ext | CTC CTC ASY CTC CTC ACT |
| V_{λ}3-Ext | GGC CTC CTA TGW GCT GAC |
| V_{λ}3I-Ext | GTT CTG TGG TTT CTT CTG AGC TG |
| V_{λ}4ab-Ext | ACA GGG TCT CTC TCC CAG |
| V_{λ}4c-Ext | ACA GGT CTC TGT GCT CTG C |
| V_{λ}5/9-Ext | CCC TCT CSC AGS CTG TG |
| V_{λ}6-Ext | TCT TGG GCC AAT TTT ATG C |
| V_{λ}7/8-Ext | ATT CYC AGR CTG TGG TGA C |
| V_{λ}10-Ext | CAG TGG TCC AGG CAG GG |
| reverse | |
| primer | |
| C_{λ}-new-ext | AGG CCA CTG TCA CAG CT |

TABLE 9
Primer pairs used for heavy in nested PCR.

| | 5′-3′ sequence |
|---|---|
| forward | |
| primer | |
| V_{H}1-Int | CTGGGTTCCAGGTTCCACTGGTGAC |
| tag* | CAG GTG CAG CTG GTR CAG TCT GGG |
| V_{H}2-Int | CTGGGTTCCAGGTTCCACTGGTGAC |
| tag | CAG RGC ACC TTG ARG GAG TCT GGT CC |
| V_{H}3-Int | CTGGGTTCCAGGTTCCACTGGTGAC |
| tag | GAG GTK CAG CTG GTG GAG TCT GGG |
| V_{H}4-Int | CTGGGTTCCAGGTTCCACTGGTGAC |
| tag | CAG GTG CAG CTG CAG GAG TCG G |
| V_{H}5-Int | CTGGGTTCCAGGTTCCACTGGTGAC |
| tag | GAR GTG CAG CTG GTG CAG TCT GGA G |
| V_{H}6-Int | CTGGGTTCCAGGTTCCACTGGTGAC |
| tag | CAG GTA CAG CTG CAG CAG TCA GGT CC |
| reverse | |
| primer | |
| IgA1-int | GC TGT GCC CCC AGA GGT |
| | GCT GGT GCT GCA GAG GCT CAG |
| IgA2-int | GC TGT GCC CCC AGA GGT |
| | GCT GGT GCT GTC GAG GCT CAG |
| IgD-int | GC TGT GCC CCC AGA GGT |
| | GTG TCT GCA CCC TGA TAT GAT GG |
| IgG-ext | GC TGT GCC CCC AGA GGT |
| | GCT CYT GGA |
| IgM-int | GC TGT GCC CCC AGA GGT |
| | GGA ATT CTC ACA GGA GAC GAG G |

*Tag sequences as highlighted in bold were used for amplification of Ig genes for creating Ig gene expression cassettes by subsequent PCR.

TABLE 10
Primer pairs used for kappa chain in
nested PCR.

| | 5′-3′ sequence |
|---|---|
| forward | |
| primer | |
| V_{κ}1-Int tag* | CTGGGTTCCAGGTTCCACTGGTGAC |
| | GAC ATC CAG WTG ACC CAG TCT C |
| V_{κ}2-Int tag | CTGGGTTCCAGGTTCCACTGGTGAC |
| | GAT ATT GTG ATG ACC CAG WCT CCA C |
| V_{κ}3-Int tag | CTGGGTTCCAGGTTCCACTGGTGAC |
| | GAA ATT GTG TTG ACR CAG TCT CCA |
| V_{κ}4-Int tag | CTGGGTTCCAGGTTCCACTGGTGAC |
| | GAC ATC GTG ATG ACC CAG TCT C |
| V_{κ}5-Int tag | CTGGGTTCCAGGTTCCACTGGTGAC |
| | GAA ACG ACA CTC ACG CAG TCT C |
| V_{κ}6-Int tag | CTGGGTTCCAGGTTCCACTGGTGAC |
| | GAA ATT GTG CTG ACW CAG TCT CCA |
| V_{κ}7-Int tag | CTGGGTTCCAGGTTCCACTGGTGAC |
| | GAC ATT GTG CTG ACC CAG TCT |
| reverse | |
| primer | |
| Cκ-int | GGG AAG ATG AAG ACA GAT GGT |

*Tag sequences as highlighted in bold were used for amplification of Ig gene expression cassettes by subsequent PCR.

TABLE 11
Primer pairs used for lambda chain in
nested PCR.

| | 5′-3′ sequence |
|---|---|
| forward | |
| primer | |
| V_{λ}1-Int | CTGGGTTCCAGGTTCCACTGGTGAC |
| tag* | CAG TCT GTG YTG ACK CAG CC |
| V_{λ}2-Int | CTGGGTTCCAGGTTCCACTGGTGAC |
| tag | CAG TCT GCC CTG ACT CAG CC |
| V_{λ}3-Int | CTGGGTTCCAGGTTCCACTGGTGAC |
| tag | TCY TAT GAG CTG ACW CAG CCA C |
| V_{λ}3I-Int | CTGGGTTCCAGGTTCCACTGGTGAC |
| tag | TCT TCT GAG CTG ACT CAG GAC CC |
| V_{λ}4ab-Int | CTGGGTTCCAGGTTCCACTGGTGAC |
| tag | CAG CYT GTG CTG ACT CAA TC |
| V_{λ}4c-Int | CTGGGTTCCAGGTTCCACTGGTGAC |
| tag | CTG CCT GTG CTG ACT CAG C |
| V_{λ}5/9-Int | CTGGGTTCCAGGTTCCACTGGTGAC |
| tag | CAG SCT GTG CTG ACT CAG CC |
| V_{λ}6-Int | CTGGGTTCCAGGTTCCACTGGTGAC |
| tag | AAT TTT ATG CTG ACT CAG CCC CAC T |
| V_{λ}7/8-Int | CTGGGTTCCAGGTTCCACTGGTGAC |
| tag | CAG RCT GTG GTG ACY CAG GAG |
| V_{λ}10-Int | CTGGGTTCCAGGTTCCACTGGTGAC |
| tag | CAG GCA GGG CWG ACT CAG |
| reverse | |
| primer | |
| C_{λ}-int | GGG YGG GAA CAG AGT GAC C |

*Tag sequences as highlighted in bold were used for amplification of Ig genes for creating Ig gene expression cassettes by subsequent PCR.

Design and Construction of the Linear Ig Expression Cassettes

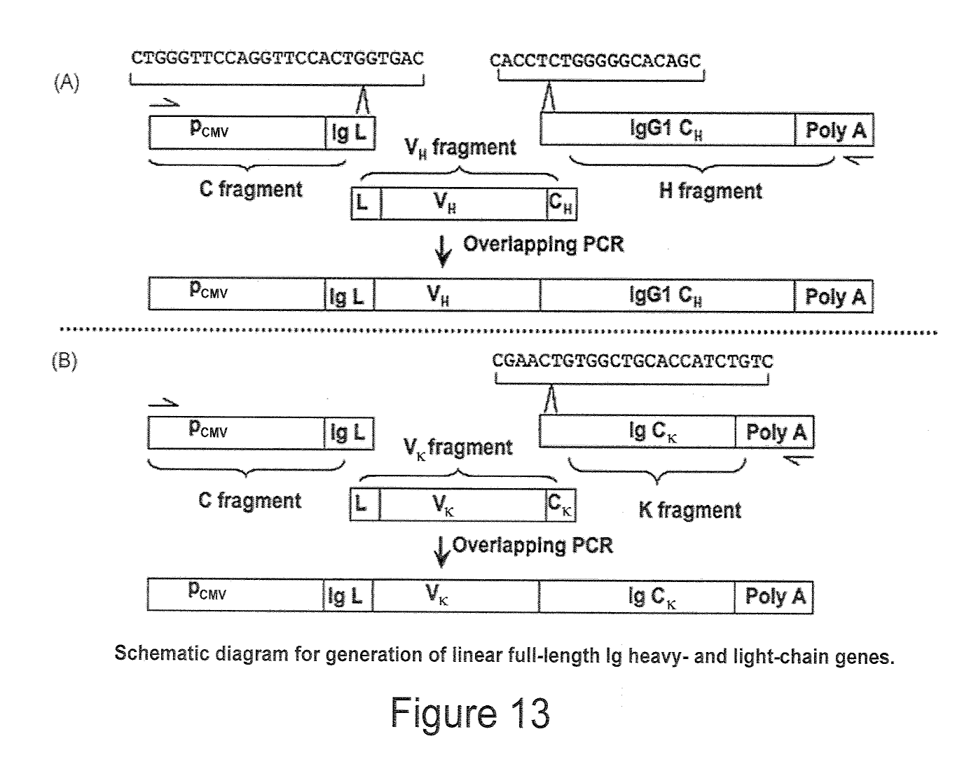

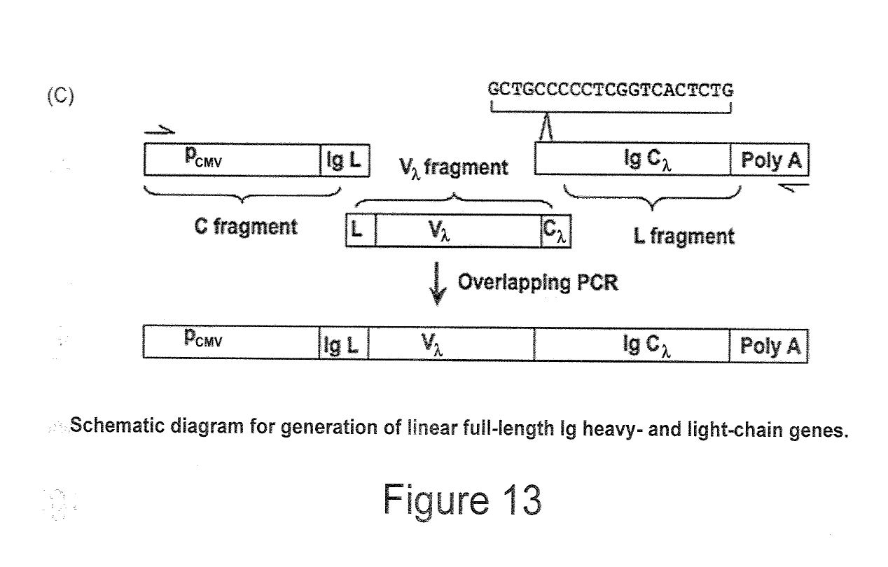

The linear Ig expression cassettes were assembled by overlapping PCR for facilitating high throughput testing of the Ig V_{H} and V_{L} genes for antibody expression and specificity analysis without cloning steps (FIG. 13). Each cassette was PCR-amplified from 3 overlapping DNA fragments including 1.) the C fragment made of the CMV promoter (705 bp) (Boshart et al., 1985) and sequence encoding for an Ig leader (METDTLLLWVLLLWVPGSTGD) (Burstein, 1978), 2.) either the H fragment (1,188 bp) made of the IgG1 constant region (315 aa) (Genbak Accession no. BC041037) (Strausberg et al., 2002) and bovine growth hormone (BGH) poly(A) signal sequences (Gimmi et al., 1989), K fragment (569 bp) made of the Ig kappa constant region (107 aa) (Strausberg et al., 2002) (GenBank Accession no. BC073791) and BHG poly(A) signal sequences, or L fragment (552 bp) made of the Ig lambda constant region (102 aa) (GenBank Accession no. BC073769) (Strausberg et al., 2002) and BGH poly(A) signal sequences (Gimmi et al., 1989) and 3.) either the V_{H}, V_{κ} or V_{λ} genes amplified from single B cells as described above (FIG. 13). The linear Ig gene cassettes contained the 5′ end restriction enzyme (Nhe I) site between the CMV promoter and Ig leader and the 3′ end restriction enzyme (Xba I) site between the Ig constant region stop codon and the poly(A) signal sequence (FIG. 13). The purpose of these restriction enzyme sites was for potential cloning of Ig genes into expression plasmids for development of stable cell lines to produce recombinant antibodies of interest.

The C, H, K and L fragments were de novo synthesized (Blue Heron, Bothell, Wash.) and cloned into pCR2.1 plasmids (Invitrogen, Carlsbad, Calif.) resulting in plasmids HV0024, HV0023, HV0025 and HV0026, respectively. For use in assembling linear Ig gene cassettes, these DNA fragments were generated from these plasmids by PCR using the primers as shown in Table 12. The PCR was carried out in a total volume of 50 μl with 1 unit of AccuPrime pfx polymerase (Invitrogen, Carlsbad, Calif.), 5 μl of 10×AccuPrime PCR buffer, 1 ng plasmid, and 10 pmol of each primer. The PCR cycle conditions were one cycle at 94° C. for 2 min, 25 cycles of a denaturing step at 94° C. for 30 s, an annealing step at 60° C. for 30 s, an extension step at 68° C. for 40 s for the C, K and L fragments or 80 s for the H fragment, and one cycle of an additional extension at 68° C. for 5 min.

TABLE 12
Tag sequence added to primers and primers used for generating Ig variable
region genes and overlapping DNA fragments.

| Forward | | Reverse | | Used to |
|---|---|---|---|---|
| primer | Tag sequence, 5′-3′ | primer | Tag sequence, 5′-3′ | amplify |
| CL-F681 | TCTGGGTTCCAGGTTCCACTGGTGAC | H-R474 | GCTGTGCCCCCAGAGGTG | V_{H} |
| CL-F681 | TCTGGGTTCCAGGTTCCACTGGTGAC | K-R405 | GACAGATGGTGCAGCCACAGTTCG | V_{κ} |
| CL-F681 | TCTGGGTTCCAGGTTCCACTGGTGAC | L-R400 | CAGAGTGACCGAGGGGGCAGC | V_{λ} |
| CMV-F262 | AGTAATCAATTACGGGGTCATTAGTTCATAG | C-R942 | GTCACCAGTGGAACCTGGAACCCAG | C fragment |
| CH-F01 | CACCTCTGGGGGCACAGC | BGH-R1235 | TCCCCAGCATGCCTGCTATTGTC | H fragment |
| K-F391 | CGAACTGTGGCTGCACCATCTGTCTTCATC | BGH-R1235 | TCCCCAGCATGCCTGCTATTGTC | K fragment |
| L-F409 | TGCCCCCTCGGTCACTCTGTTCCCGCCC | BGH-R1235 | TCCCCAGCATGCCTGCTATTGTC | L fragment |

The linear full-length Ig heavy- and light-chain gene expression cassettes were assembled by PCR from the C, V_{H} and H fragments for heavy-chain, the C, V_{κ} and K fragments for kappa chain, and the C, V_{λ} and L fragments for lambda chain (1 ng of each). The PCR reaction was carried out in a total volume of 50 μl with 1 unit of KOD DNA polymerase (Novagen, Gibbstown, N.J.), 5 μl of polymerase 10×PCR buffer, 200 μM of dNTP, 10 pmol of 5′ primer CMV-F262 and 3′ primer BGH-R1235 (Table 12). The PCR cycle program consisted of one cycle at 98° C. for 1 min, 25 cycles of a denaturing step at 98° C. for 15 s, an annealing step at 60° C. for 5 s, an extension step at 72° C. for 35 s and one extension cycle for 10 min at 68° C.

Expression of Recombinant Antibodies

PCR products of the linear Ig expression cassettes were purified using a Qiagen PCR Purification kit (Qiagen, Valencia, Calif.). The purified PCR products of the paired Ig heavy- and light-chain gene expression cassettes were co-transfected into 80-90% confluent 293T cells grown in 12-well (1 μg of each per well) tissue culture plates (Becton Dickson, Franklin Lakes, N.J.) using PolyFect (Qiagen, Valencia, Calif.) and the protocol recommended by the manufacturer. Plasmids HV13221 and HV13501 (1 μg of each per well) expressing Ig heavy or light-chain genes derived from the 2F5 mAb were used under the same conditions as positive controls. Six to eight hours after transfection, the 293T cells were fed with fresh culture medium supplemented with 2% FCS and were incubated for 72 hours at 37° C. in a 5% CO₂ incubator.

ELISA to Determine the Specificity and Quantity of Antibodies

To measure the concentration of recombinant mAbs in transfected culture supernatants, mouse anti-human Ig (Invitrogen, Carlsbad, Calif.) at 200 ng/well was used to coat 96-well high-binding ELISA plates (Costar/Corning; Lowell, Mass.) using carbonate bicarbonate buffer at pH 9.6. Plates were incubated overnight at 4° C. and blocked at room temperature (RT) for 2 hours with PBS containing 4% wt/vol whey protein, 15% goat serum, 0.5% Tween-20, and 0.05% NaN₃. 100 μL of supernatant from transfected cell cultures or control human IgG1 antibodies were incubated at RT for 2 hours. Goat-anti-human IgG specific (heavy- and light-chain)-alkaline phosphatase (AP) (1:3000 dilution) (Sigma, St. Louis, Mo.) diluted in blocking buffer was used as the secondary antibody and incubated at RT for 1 hour. For color development, the AP substrate was 2 mM MgCl₂ and 1 mg/ml 4-nitrophenyl phosphate di(2-amino-2-ethyl-1,3-propanediol) salt in 50 mM Na₂CO₃ buffer (pH 9.6), was added and incubated for 45 minutes. Plates were read in an ELISA reader at 405 nm. Amounts of IgG secreted in the transfected 293T cells were determined by comparison to a standard curve generated using known concentration of the control human IgG1.

Similar ELISA procedures as described above were used for detecting the binding of recombinant mAbs to specific antigens. Antigens for detection of anti-HIV-1 antibodies included HIV-1 Env MPER peptide, SP62 (QQEKNEQELLELDKWASLWN) (Alam et al., 2008), HIV-1 Env immunodominant epitope peptide (PrimmBiotech, Cambridge, Mass.), SP400 (RVLAVERYLRDQQLLGIWGCSGKLICTTAVPWNASWSNKSLNKI) (CPC Scientific, San Jose, Calif.), SP62-scrambled peptide (NKEQDQAEESLQLWEKLNWL) as a negative control (Alam et al., 2008), HIV-1 gp41 and HIV-1 JRFL gp140 protein (Liao, 2006). Fluzone® 2007-2008 (Sanofi Pasteur, Lyon, France), a trivalent inactivated influenza vaccine containing an A/Solomon Islands/3/2006 (H₁N₁)-like virus, an A/Wisconsin/67/2005 (H₃N₂)-like virus and a B/Malaysia/2506/2004-like virus, HA of H1 A/Solomon Islands, H3 A/Wisconsin, H3 A/Johannesburg and H5 A/Vietnam (Protein Sciences; Meriden, Conn.) were used as coating antigens in ELISA for detection of anti-influenza antibodies. Individual antigens at 200 ng/well were used to coat 96-well high-binding ELISA plates.

SDS-polyacrylamide Gel Electrophoresis and Western Blot Blot Analysis of Expressed Recombinant mAb

Transfected culture supernatant samples (16 μl per lane) and controls were fractionated on precasted 4-12% Bis-Tris SDS-PAGE gels (Invitrogen, Carlsbad, Calif.) under non-reducing conditions, transferred onto nitrocellulose filters and probed with goat-anti-human IgG specific (heavy- and light-chain)-AP (1:3000 dilution) (Sigma, St. Louis, Mo.). The immunoblots were developed with Western-blue substrate (Promega; Madison, Wis.).

### Results

Expression of V_{H} and V_{L} Genes Without Cloning

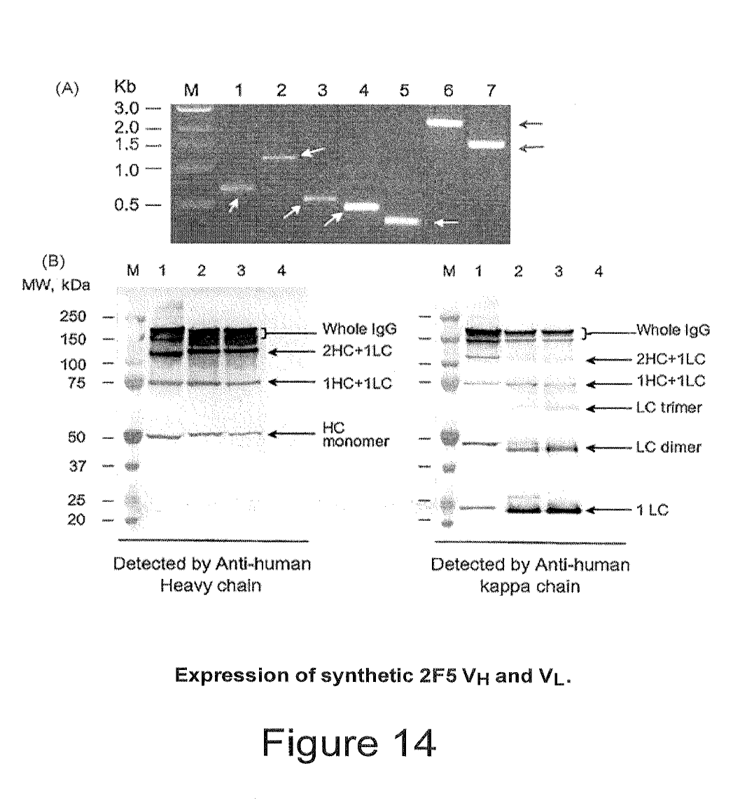

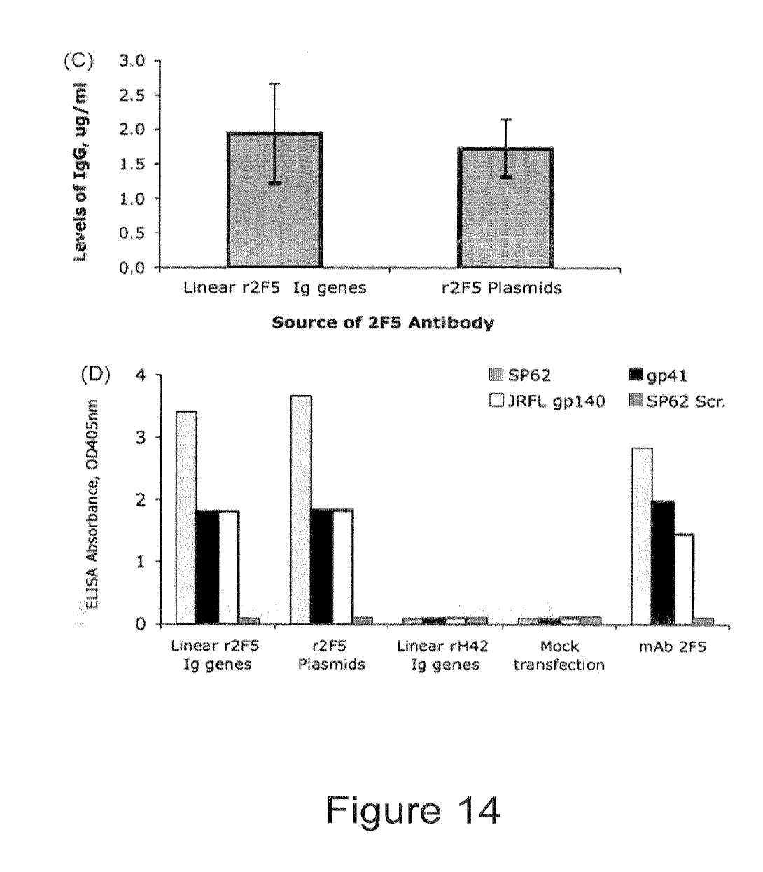

Synthetic recombinant mAb 2F5 V_{H} and V_{L} genes (Ofek et al., 2004) were used as a model system for method development. Synthetic IgG1 heavy-chain and kappa chain genes were first cloned into pcDNA3.1/hygro plasmids and used to produce functional r2F5 HV01 mAb by stable transfection. Purified r2F5 HV01 mAb was compared with mAb 2F5 Polymun for their neutralizing activity in pseudotype HIV-1 neutralization assays (Montefiori, 2005). It was found that the recombinant 2F5 neutralized HIV-1 isolates with a similar potency as the commercial mAb 2F5 (Table 13). Next, the 2F5 V_{H} and V_{L} genes were amplified from 2F5 Ig heavy- and light-chain plasmids using the primer pair of CL-F681 and H-R474 for V_{H} and the pair of CL-F681 and K-R405 for V_{L} as shown in Table 12. Assembly of 2F5 V_{H} and V_{L} genes into linear Ig gene cassettes was performed by overlapping PCR of the 2F5 V_{H} and V_{L} genes and the C, H and K, DNA fragments, and analyzed using agarose gel electrophoresis (FIG. 14A). A pair of V_{H} and V_{L} genes (rH42) isolated from a sorted single B cell were used as negative controls and assembled into the linear full-length Ig gene cassettes using the same procedure as for 2F5 Ig genes. The linear full-length 2F5 heavy- and light-chain gene cassettes were co-transfected into 293T cells. Plasmids HV13221 expressing the 2F5 heavy-chain gene and HV13501 expressing the 2F5 light-chain gene were used in parallel cultures as positive controls during transfection. The culture supernatants of the transfected-293T cells were harvested 3 days after transfection, analyzed by Western blot for the presence of Ig (FIG. 14B), and assayed by ELISA to measure the concentration of IgG (FIG. 14C) and determine the specificity of Igs against the HIV-1 Env MPER peptide SP62 (Alam et al., 2008), HIV-1 gp41 and HIV-1 JRFL gp140 (FIG. 14D). Co-transfection of the 2F5 heavy- and light-chain genes in the form of either plasmids or linear Ig gene cassettes not only produced whole IgG molecules with molecular weights of 150 kDa as well as IgG molecules containing extra heavy- or light-chains with molecular weights of more than 150 kDa as detected by both anti human Ig heavy- and light-chain antibodies (FIG. 14B). As shown in FIG. 14B, co-transfection of the 2F5 heavy- and light-chain genes also produced 1) mixtures of monomers of Ig heavy-chains (50 kDa) detected only by an anti-human heavy-chain antibody; 2) monomers (23 kDa), dimers (46 kDa) and trimers (69 kDa) of light-chains detected by an anti-light-chain antibody and 3) Ig molecules with different combinations (1:1 or 2:1 ratio) of heavy- and light-chains. From six independent transfection experiments, the average amounts of IgG produced by 293T cells transfected with the linear synthetic 2F5 heavy- and light-chain Ig gene cassettes were comparable to that produced in 293T cells transfected with plasmids of the 2F5 heavy- and light-chain genes (1.9 μg/ml IgG+0.7 μg/ml (mean±SEM), n=6 versus 1.7 μg/ml+0.4 μg/ml, n=6, respectively) (FIG. 14C). As expected, similar to commercial mAb 2F5, the recombinant 2F5 IgG antibodies produced in 293T cells by transfection with either the linear 2F5 Ig gene cassettes or plasmids expressing 2F5 Ig genes reacted with HIV-1 MPER peptide SP62, HIV-1 gp41 and HIV-1 gp140 proteins but did not react with the negative control scrambled SP62 peptide (FIG. 14D). Supernatants generated by transfection of 293T cells with linear Ig heavy- and light-chain cassettes from the control antibody (rH42) and supernatants from mock-transfected 293T cells did not react with any of the HIV-1 proteins or peptides (FIG. 14D).

TABLE 13
HIV-1 neutralization activity of mAb 2F5
and recombinant (r) 2F5 antibody.

| | 50% neutralization level (ug/ml) | | |
|---|---|---|---|
| | against HIV-1 Isolates | | |
| Antibody | B.SF162 | B.BG1168 | C.TV-1 |
| mAb 2F5 | 0.1 | 0.32 | 10.87 |
| r2F5 | 0.3 | 1.17 | 34.48 |

Expression of Ig V_{H} and V_{L} Genes Derived from Cloned EBV-transformed B Cell Lines

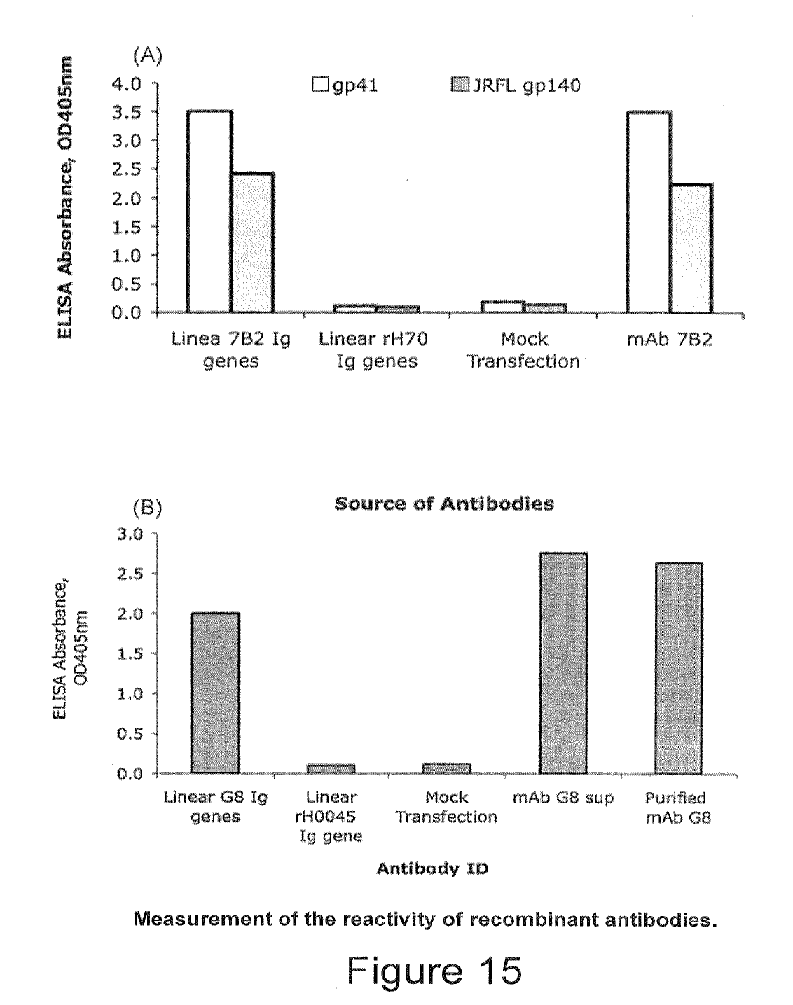

A major problem with available techniques for EBV transformation of B cells for generation of human mAbs is the low rate of B cell clone rescue. To determine whether the utility of the Ig linear cassette method for isolation and functional characterization of Ig genes could be used for rapid Ig gene profiling of EBV transformed B cells, this approach was tested on two cloned EBV-transformed human B cell lines, 7B2 (Binley et al., 2000) and G8 (Hwang, unpublished), that produce mAbs against HIV-1 gp41 and HIV-1 Env immunodominant epitope, respectively. Ig sequence information was not available from the 7B2 and G8 cell lines, therefore, the V_{H} and V_{L} genes of 7B2 and G8 were amplified using the RT-PCR method as described above. It was found that the Ig genes for 7B2 consisted of an IgG1 heavy-chain and a kappa a0 light-chain and the Ig genes for 08 consisted of an IgG1 heavy-chain and a lambda light-chain. Assembly of the 7B2 and G8 V_{H} and V_{L} genes into linear full-length Ig gene cassettes was performed by overlapping PCR using the same method as for 2F5 Ig genes. The resulting linear Ig gene cassettes were transfected into 293T cells for expression of recombinant mAbs, By ELISA, the recombinant 7B2 IgG antibodies produced by transfection using linear Ig gene cassettes performed just like the mAb produced by the 7B2 EBV-transformed B cell line. Both preparations of 7B2 mAb reacted with HIV-1 gp41 and gp140 proteins, while the control antibody (rH70) or supernatant of mock-transfected 293T cells was non-reactive with these same proteins (FIG. 15A). Similar results were obtained using linear Ig gene cassettes generated from the G8 human B cell line (FIG. 15B). These results demonstrated that the linear Ig gene cassette method could be used to produce mAbs from B cells.

Isolation and Expression of Ig V_{H} and V_{L} Genes Derived from Sorted Single Plasma Cells

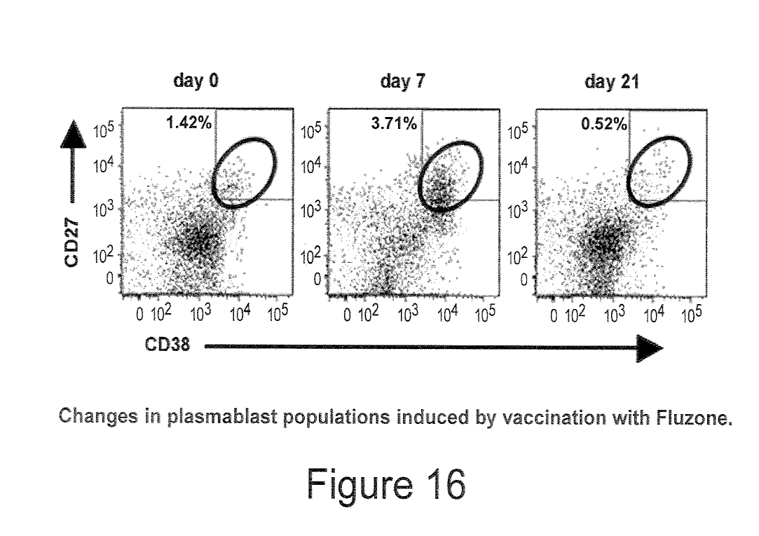

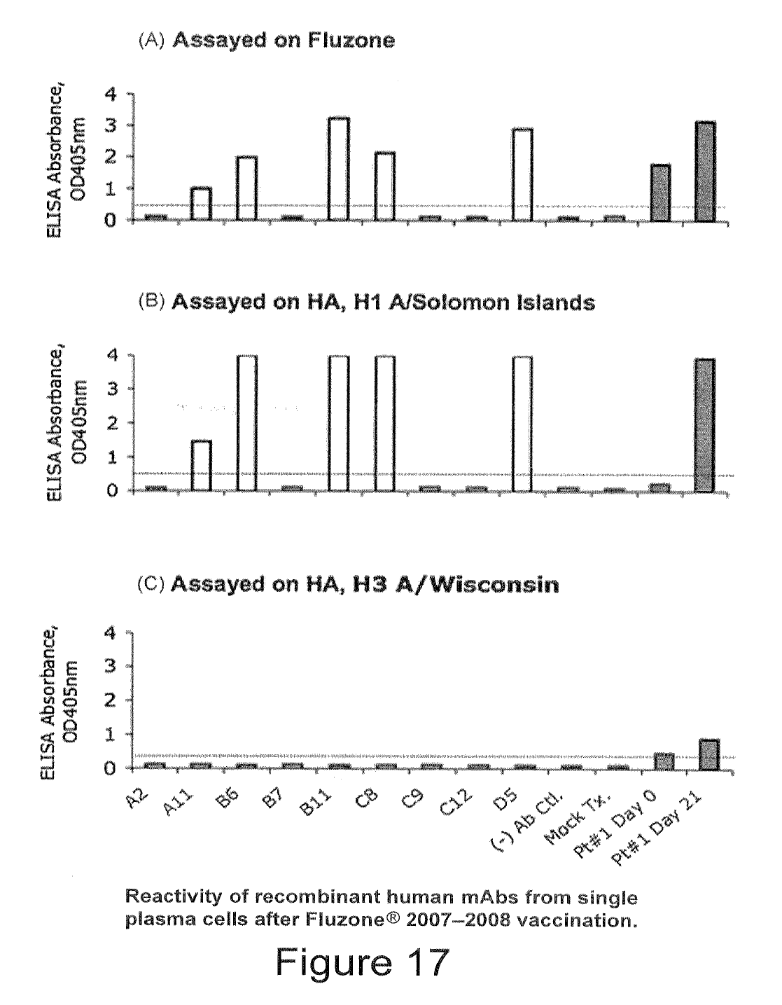

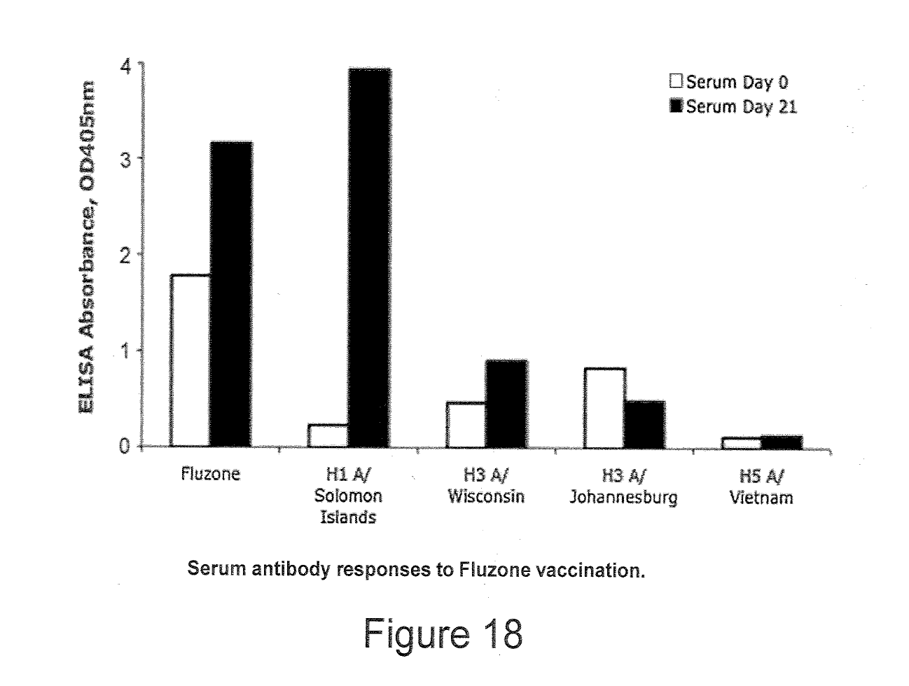

To demonstrate the utility of linear Ig gene cassettes for producing and screening mAbs from the V_{H} and V_{L} genes from sorted single primary human B cells, this strategy was tested using plasmablasts from a subject immunized with killed influenza vaccine Fluzone® 2007-2008. PBMC were isolated from a subject at day 0, 7 and 21 post-vaccination with Fluzone® 2007-2008 and were analyzed by flow cytometry. It was found that at day 7 after the Fluzone® vaccination, peripheral blood cells with a plasmablast phenotype (CD19⁺, CD20low-neg, CD27⁺⁺ and CD38⁺⁺) were increased compared to baseline (day 0); plasmablasts returned to baseline by day 21 after vaccination (FIG. 16). These results were consistent with studies reported by Wrammert and colleagues (Wrammert et al., 2008). Using BSL-3 BD FACSAria-based preparative cell sorting, single plasma cells from day 7 PBMC were sorted into 96-well plates (Wrammert et al., 2008). Nine Ig V_{H} and V_{L}, pairs were isolated from day 7 plasmablasts by RT-PCR amplification of 24 wells of sorted single cells. The Ig V_{H} and V_{L} pairs were assembled into linear Ig gene cassettes and used to produce mAbs in 293T cells by transient transfection. It was found that 5 of the 9 recombinant mAbs were strongly reactive high-affinity anti-HA mAbs that reacted with inactivated influenza viruses in Fluzone® 2007-2008 (FIG. 17A) and with H1 A/Solomon Islands hemagglutinin (HA) (FIG. 17B) but not with H3 A/Wisconsin HA (FIG. 17C) that was also in the vaccine. The Ig concentration of these 5 antibodies ranged from 0.2 μg/ml to 1.3 μg/ml in the transfected culture supernatants. Sequence analysis of the V_{H} and V_{L} genes indicated that these five HA binding antibodies were distinct from each other (Table 14). The antibody B6 was an IgA antibody and the other 4 HA binding antibodies were IgG (Table 14). Importantly, the spectrum of the reactivity of these antibodies was reflective of serum antibody responses in the vaccinee. ELISA assays on serum samples collected at day 0 and 21 days after vaccination showed that there were preexisting, high levels of antibody to Fluzone® 2007-2008 and only low levels of antibody to H1 A/Solomon Islands HA or to H3 A/Wisconsin HA (FIG. 18). As such, Fluzone® 2007-2008 vaccination boosted antibody responses to the Fluzone®-2007-2008 and to A/Solomon Islands HA, and only weakly boosted antibody responses to H3 A/Wisconsin HA (FIG. 18). Not only did the results of the Fluzone® 2007-2008 plasmablast analysis support the utility of the linear Ig gene cassette method for producing human antibodies from human B cells but these results also demonstrated that the reactivity of human mAbs produced by using this method reflects the range of human antibody responses in this subject.

TABLE 14
Variable regions of Ig heavy and light chain genes isolated from
sorted single plasmablasts that reacted with Fluzone and H1 HA.

| Anti- | V_{H} | | | | V_{L} | | |
|---|---|---|---|---|---|---|---|
| body | | | CDR3 | Ig | | | CDR3 |
| ID | V_{H} ID | Family | Length | Isotype | V_{L} ID | Family | Length |
| A11 | H0076 | 4-402 | 23 | G | K0069 | 1-39 | 10 |
| B6 | H0077 | 3-30 | 17 | A | K0070 | 3-20 | 10 |
| B11 | H0079 | 3-43 | 17 | G | L0020 | 1-44 | 11 |
| C8 | H0080 | 4-39 | 19 | G | L0021 | 6-57 | 11 |
| D5 | H0082 | 2-04 | 19 | G | L0024 | 3-21 | 11 |

### Discussion

In this study, a novel system was tested for Ig gene expression without prior cloning of V_{H} and V_{L} genes into expression vectors. In vitro expression of rearranged Ig genes as antibodies, requires cloning of amplified Ig V_{H} and V_{L} into eukaryotic cell expression plasmids containing a transcription regulation control element such as the CMV promoter, an Ig leader sequence, a poly(A) signal sequence and the constant region of the Ig heavy- or light-chain (Persic et al., 1997; Tiller et al., 2008; Wrammert et al., 2008). Several Ig expression vectors have been developed that produce functional Ig (Norderhaug et al., 1997; Persic et al., 1997; McLean et al., 2000; Tiller et al., 2008). However, cloning procedures are often the bottleneck for expression of recombinant antibodies for antibody selection. Here, functional linear Ig gene cassettes assembled from three DNA fragments with overlapping sequences by PCR were described. The feasibility of the Ig production approach was demonstrated in 3 ways. First, the V_{H} and V_{L} genes derived from the anti-HIV-1 gp41 mAb 2F5 were used to produce functional r2F5 HV01 mAb. Second, it was demonstrated that the linear Ig gene cassette method could be used to produce functional HIV-1 antibodies from 2 EBV transformed cell lines, thus providing a powerful method of rescue of human mAbs from EBV-transformed B cell cultures. Finally, it was demonstrated that the linear Ig gene cassette method could be used to produce functional antibodies that bind influenza HA from peripheral blood plasmablasts from subjects vaccinated for influenza.

The linear Ig gene cassettes described herein contain all the essential elements necessary to produce functional antibodies. The cassettes contain a promoter (Boshart et al., 1985), Ig leader (Burstein, 1978), the constant region of IgG1 heavy-chain (Strausberg et al., 2002) or Ig light-chains (kappa and lambda) (Strausberg et al., 2002), poly(A) tail (Gimmi et al., 1989) and V_{H} or V_{L} genes. The V_{H} and V_{L} genes can be easily substituted with any V_{H} and V_{L} genes of humans, mouse or other origin (data not shown). Given the different forms of V_{H} and V_{L} that might be derived from different sources such as human or mouse, guidelines for designing the primers have been given in Table 12 for creating the overlapping sequences. The constant region of the linear Ig heavy-chain gene cassette was derived from IgG1 because IgG1 is the most common Ig isotype among all Ig types. It was demonstrated that the chimeric IgG1 antibodies derived from B cells that expressed IgG (G8 and 7B2) had the same specificity and similar binding affinity as the original antibodies. Importantly, functional linear Ig gene cassettes produced Ig by transient transfection in 293T cells at levels that were comparable to that produced by transfection with plasmid DNA (FIG. 14) or by EBV-transformed B cell lines (FIG. 15B). The amounts (1 ml per well) of antibody samples generated in 12-well plates by transfection with linear Ig gene cassettes would be sufficient for most binding or neutralization assays, especially in multiplexed luminex systems (Croft et al., 2008) or antigen microarrays (Robinson, 2006).

The isolation of V_{H} and V_{L} genes from sorted single cells makes it possible to analyze Ig genes from single B cells and to produce recombinant mAbs (Babcook et al., 1996; Wardemann et al., 2003; Volkheimer et al., 2007; Tiller et al., 2008; Wrammert et al., 2008). The analysis of single B cells and linkage of the Ig reactivity profile with Ig gene sequences can provide valuable insight into the molecular basis of Ig gene rearrangement, allelic exclusion and Ig selection in the antibody repertoire (Kuppers et al., 1993; Brezinschek et al., 1995; Babcook et al., 1996; Wang and Stollar, 2000; Owens et al., 2003). Sorting of single cells into 96-well PCR plates followed by RT-PCR has been demonstrated as a very efficient process for isolation of small numbers of single cells with paired V_{H} and V_{L} genes (Tiller et al., 2008; Wrammert et al., 2008). By using the linear Ig expression cassettes method, it took only 6 working days from the time of flow cytometry analysis and single cell sorting of the PBMC from an influenza vaccinee to obtain five recombinant mAbs that were specific for influenza viruses. For production of mAb-expressing cell lines by stable transfection, once mAbs are obtained with the desired specificity, the Ig gene expression cassettes can be readily cloned into an expression plasmid like pcDNA3.3-TOPO using TA cloning (Invitrogen, Carlsbad, Calif.) or pcDNA3.1 (Invitrogen, Carlsbad, Calif.) using restriction enzyme digestion-ligation, because the Ig gene expression cassettes were designed to contain unique Nhe I-Xbo I sites (5¹-3) that are extremely rare cutters for Ig genes (Persic et al., 1997) of the full-length of Ig heavy- and light-chain constructs (FIG. 13).

Thus, by combining the isolation of Ig V_{H} and V_{L} genes from single cells by RT-PCR (Tiller et al., 2008; Wrammert et al., 2008) and the use of novel linear Ig gene expression cassettes described here, a rapid strategy for expressing Ig genes was developed for screening and analysis within days of B cell isolation. Importantly, this system has the advantage that it can be scaled up for high-throughput human mAb production as we have recently generated more than 600 recombinant antibodies derived from sorted human plasmablasts by using this approach for screening against HIV-1 and other antigens to profile B cells responses to acute HIV-1 infection (manuscript in preparation, H-X Liao and B. F. Haynes). This strategy could also be adapted to generate recombinant high affinity human or non-human antibodies, for use as therapeutic agents, for development of mutant antibodies, for use in mechanistic studies of antibody-antigen interactions, and for rescuing antibodies from EBV-transformed cell lines or mycoplasma-contaminated antibody-producing B cell or hybridoma cell lines.

### Example 3

### Experimental Details

Human Samples:

Stored plasma and PBMC from CAP206 an HIV-1 subtype C chronically infected individual were used for this study. This participant is part of the CAPRISA 002 Acute infection cohort whose antibody neutralization profile has been studied since the point of seroconversion (Gray et al, J. Virol. 81:6187-6196 (2007)). This study was approved by the IRB of the Universities of KwaZulu Natal and Witwatersrand in South Africa.

Reagents:

The MPR.03 peptide containing lysines at both ends for solubility (KKKNEQELLELDKWASLWNWFDITNWLWYIRKKK-biotin) and a scrambled peptide were used to generate tetramers. Other peptides (MPER656, SP62, SP400 and 4E10) and proteins (ConS gp140, JR-FL and gp41) were used in ELISAs and SPR experiments and have been described previously (Shen et al, J. Virol. 83:3617-3625 (2009)). 4E10 and 2F5 mAbs were used as controls. The CAP206.B5 transmitted/founder virus was cloned from an early plasma sample. Other viruses are from the standard Glade B and C panels.

Preparation of Tetramers:

Tetramers were prepared using the biotinylated MPR.03 peptide with both allophycocyanin (APC) and Pacific Blue labelled streptavidins and titered on antibody-coated beads and on antibody expressing cell lines (using the 13H11 and 2F5 mAbs which both bind the MPR.03 peptide). Briefly, excess biotinylated peptide (approximately 33:1 molar ratio of peptide to streptavidin for fluorochrome-labeled tetramers) was incubated at 4° C. overnight and isolated using gel filtration on Micro BioSpin 30 columns, Tetramers were assayed for final concentration determined using standard spectrophotometric techniques. Final titers were determined using a combination of 2F5-coated beads and 13H11-expressing cell lines. Tetramers were used in equimolar amounts in combination with a panel of monoclonal antibodies to identify memory B cells in PBMC

Staining and Sorting B cell Populations:

Thawed PBMC were stained with a combination of the following antibodies: CD3 PE-Cy5, CD14 PE-Cy5, CD16 PE-Cy5, CD235a PE-Cy5, CD19 APC-Cy7, CD27 PE-Cy7, CD38 APC-Cy5.5 and IgG-PE (BD Biosciences, Mountain View, Calif. and Invitrogen, Carlsbad, Calif.). All antibodies were titered and used at optimal concentrations for flow cytometry. Memory B cells were gated as CD3−, CDI4−, CD16−, CD235a−, CD19+, CD27hi, CD38low and IgG+. Tetramer-stained B cells were sorted as single cells into wells of a 96-well plate, selecting those cells that were labelled by both tetramers. Cells were stored in RT reaction buffer at −80° C. until use. Flow cytometric data was acquired on a BD FACS Aria and the data analyzed using FlowJo.

Isolation of Ig Variable Gene Transcripts:

The genes encoding V_{H} and V_{L} were amplified by PCR using a modification of the method described by Tiller and co-workers (Tiller et al., 2008), Briefly, RNA from single sorted cells was reverse transcribed using Superscript III in the presence of primers specific for human IgG, IgM, IgD, IgA1, IgA2, kappa and lambda constant gene regions (Liao et al., 2009). The V_{H}, V_{K} and V_{L} genes were then amplified from this cDNA separately in a 96-well nested PCR as described and analysed on 1.2% agarose gels (Liao et al., 2009). The second round PCR includes tag sequences at the 5′ end of each primer which permits assembling of the V_{H} and V_{L} genes into functional linear Ig gene expression cassettes (see below). PCR products were purified and sequenced. The variable gene segments and potential functionality of the immunoglobulin was determined using the SoDA program (Volpe et al., 2006).

Expression of Recombinant Antibodies from Linear Expression Cassettes:

Three linear Ig expression cassettes each containing the CMV promoter and human Ig leader as one fragment were used for small-scale expression and specificity analysis (Liao et al., 2009). Fragments for the heavy and light chains comprised either the IgG1 constant region, Ig kappa constant region or Ig lambda constant region attached to poly A signal sequences. These two fragments plus either V_{H}, V_{K} or V_{L} genes amplified from single B cells as described above were assembled by overlapping PCR. PCR products containing linear full-length Ig heavy- and light-chain genes were purified and the paired Ig heavy and light-chain products co-transfected into 293T cells grown in 12-well plates using Fugene. Cultures were fed 6-12 hrs later with ˜2 mls fresh medium containing 2% FCS and incubated for 72 hours at 37C in a 5% CO2 incubator. Thereafter, culture supernatants were harvested for antibody characterization.

Design and Synthesis of Inferred Unmutated Common Ancestor and Phylogenetic Intermediate Antibodies.

SoDA program (Volpe et al., 2006) was used to infer the reverted unmutated common ancestor (RUA) VH and VL genes of CAP206-CH12, These inferred RUA V_{H} and V_{L} genes were synthesized (GeneScript, Piscataway, N.J.) and cloned as full-length IgG1 for heavy chain and full-length kappa light chain genes into pcDNA3.1 plasmid (Invitrogen; Carlsbad, Calif.) using standard recombinant techniques.

Production of Purified Recombinant mAbs.

The selected immunoglobulin VH and VK genes from CAP206-CH12 were cloned into human Igγ and Igκ expression vectors in pcDNA3.3 (Liao et al., 2009). Clones with the correct size inserts were sequenced to confirm identity with the original PCR product. For production of purified antibodies of CAP206-CH12 and CAP206-CH12_RU by batch transient transfections, 10-20 T-175 flasks or a Hyperflask of 293T cells grown at 80-90% confluency in DMEM supplemented with 10% FCS was co-transfected with plasmids expressing HIV-1 specific Ig heavy- and light chain genes using Fugene (Qiagen, Valencia, Calif.) Recombinant antibodies were purified using anti-human IgG heavy-chain specific antibody-agarose columns.

Antibody Specificities:

Supernatants from the small scale transfections and purified mAb were tested for reactivity using various peptides and proteins in an ELISA as described (Liao 2009). An anti-cardiolipin ELISA was used as previously described (Harris and Hughes, Sharma et al., 2003). Autoantibodies were measured by the FDA-approved AtheNA Multi-Lyte® ANA II Test Kit from Zeus Scientific, Inc. per the manufacturer's instructions and as described previously (Haynes et al, Science 308:1906-1908 (2005)).

Surface Plasmon Resonance:

MPER656, MPR.03 and a scrambled version of MPR.03 were individually anchored on a BIAcore SA sensor chip as described previously (Alam et al., 2004; Alam et al., 2007). Assays were performed on a BIAcore 3000 instrument at 25° C. and data analyzed using the BIAevaluation 4.1 software (BIAcore) (Alam et al 2007). Peptides were injected until 100-150 response units of binding to strepavidin were observed

Neutralization Assays:

The TZM-bl pseudovirus assay was used to assess the neutralization activity of CAP206-CH12 against viruses that were sensitive to CAP206 plasma antibodies as well as to a large panel of 26 unselected heterologous Tier 2 viruses from multiple subtypes. The mAb concentration at which 50% of virus neutralization is seen (IC₅₀ value) is reported. Purified mAb was used for these experiments to avoid interference from transfection reagents. The broadly neutralizing mAbs 4E10 and 2F5 were included for comparison.

### Results

CAP206 Plasma Reactivity and Labeling of MPER-Reactive Memory B Cells:

An HIV-1-infected individual was previously identified from the CAPRISA 002 acute infection cohort in Durban, South Africa who developed broadly cross-reactive neutralizing antibodies (Gray et al, J. Virol 83:8925-8937 (2009)). The plasma from this individual showed evidence of MPER-specific antibodies within 6 months of infection although these initial antibodies were non-neutralizing (Gray et al, J. Virol. 81:6187-6196 (2007)). However, at 18 months, this individual acquired the ability to simultaneously neutralize a large number of heterologous isolates largely via anti-MPER antibodies. This was shown by depleting neutralizing activity in plasma by adsorption with MPER-peptide, MPR.03 (KKKNEQELLELDKWASLWNWFDITNWLWYIRKKK). Of the 44 viruses tested against plasma collected at 3 years post-infection, 50% were neutralized of which approximately 70% were dependent on antibodies against the MPER (Gray et al, J. Virol 83:8925-8937 (2009)).

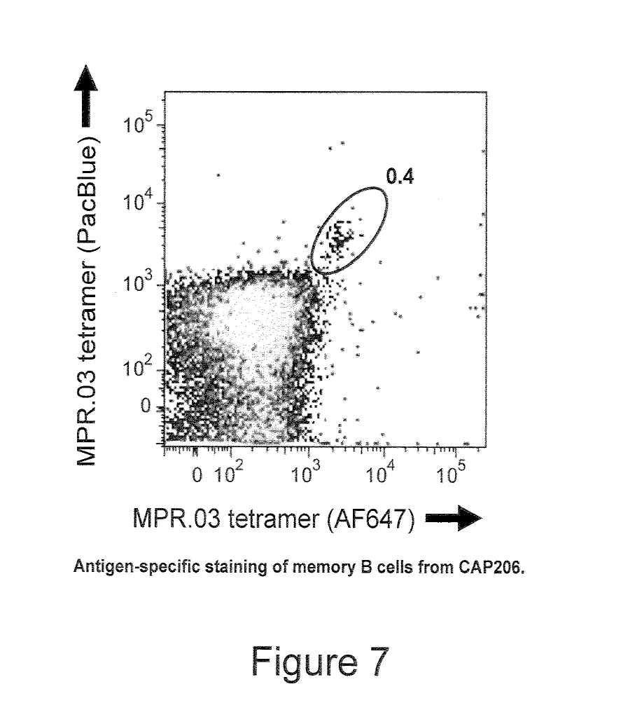

The ability to deplete specific antibodies from the plasma of CAP206 using an MPER peptide suggested that it may be possible to label and sort memory B cells producing these antibodies. A peptide tetramer was, therefore, designed based on the MPR.03 peptide. For this, the MPR.03 monomer peptide was biotinylated and reacted with streptavidin to yield a tetramer with 4 MPER epitopes for B cell surface Ig cross-linking (Verkoczy 2009). To decrease the overall labeling background, MPR.03 tetramers were labeled with either AF647 or PacBlue and used to stain PBMC from CAP206 collected at 28 months post-infection after the development of broadly neutralizing antibodies. Memory B cells (CD19+, CD27+) that were dual stained with both MPR.03-PacBlue and MPR.03-AF647 were sorted into individual wells of a 96 well plate (FIG. 7). The frequency of tetramer-specific B cells was approximately 40/10,000 of memory B cells. Given that memory B cells constituted ˜1-2% of this sample, it was estimated that the peptide-binding B cells represented ˜1 in 10,000 of total PBMC.

Isolation of HIV-1 Env gp41 MPER-Reactive mAb:

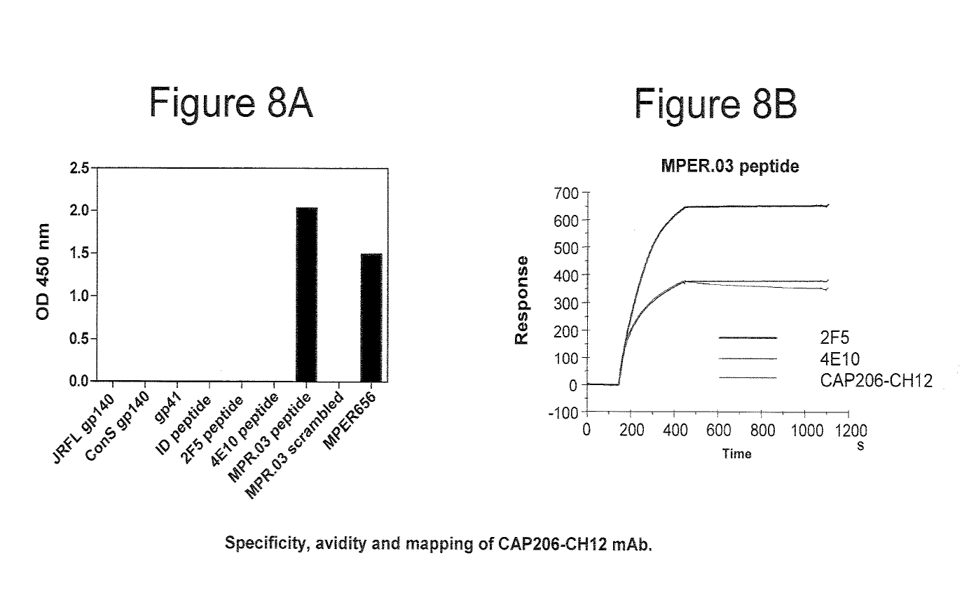

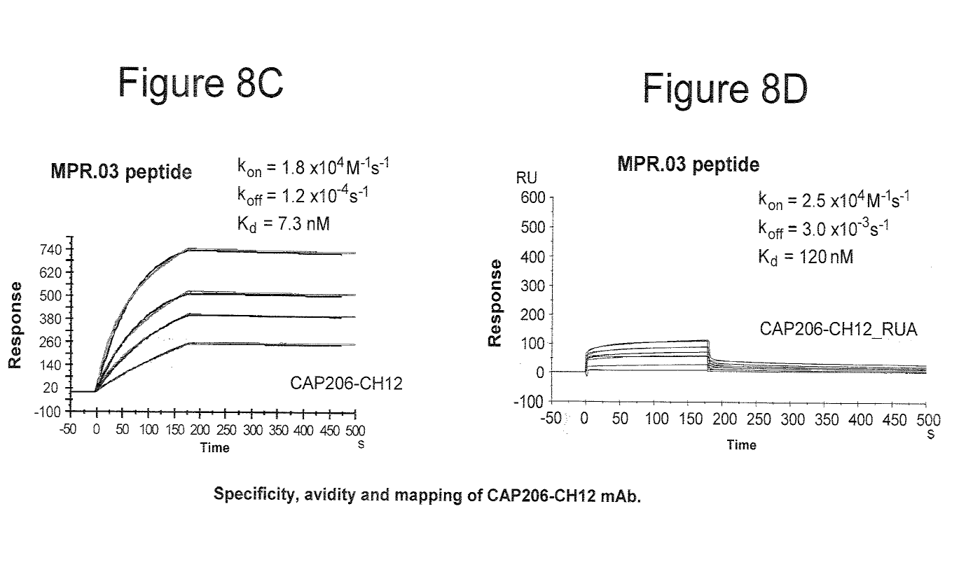

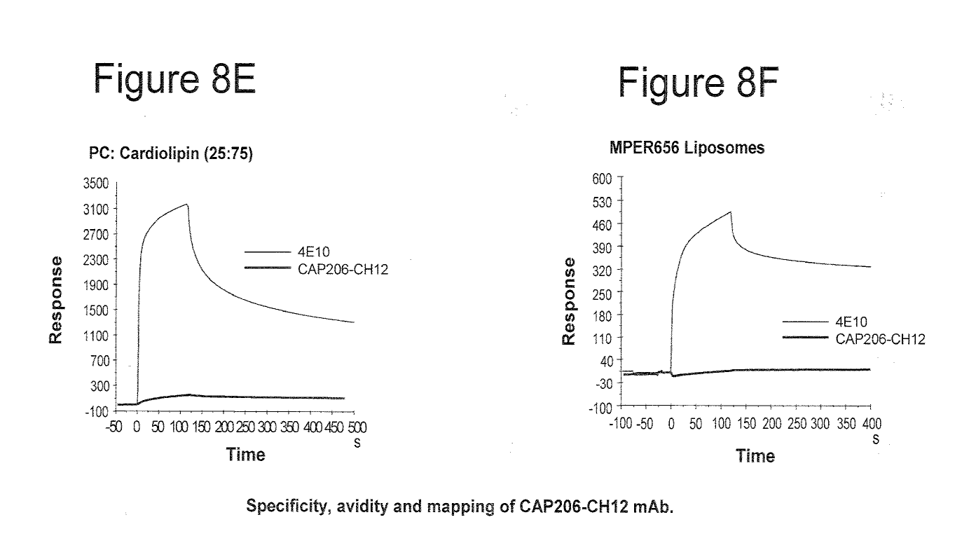

Single cell PCR amplification and transient expression of immunoglobulin (Ig) genes of sorted B cells yielded an IgG1 mAb, CAP206-CH12 that reacted strongly with the MPR.03 and MPER656 (NEQELLELDKWASLWNWFNITNWLW) but not scrambled peptides in ELISA (FIG. 8A). This mAb did not react with the clade B recombinant gp140 JRFL envelope protein nor with the group M consensus Env protein. The gp41MPER sequences in both JRFL and ConS gp140 were similar to MPR.03/656 sequences, suggesting that lack of reactivity was due to occlusion of the MPER in gp140.

Characterization of Binding Site and Affinity of CAP206-CH12:

CAP206-CH12 mAb binds to MPER.03 peptide with a binding Kd of 7.3 nM (FIG. 8) which is comparable to those of 4E10 mAb binding to MPER peptides. Alanine scanning studies showed that CAP206-CH12 binding epitope spans the WF(N/D)IT motif, which overlaps with both 4E10 and Z13e1 epitopes. With the exception of T676A (˜30% reduced), all other substitution of residues within the epitope reduced. CAP206-CH12 binding by >50% relative to the wild type peptide. Although the CAP206-CH12 epitope includes two critical residues of 4E10 epitope, W⁶⁷² and F⁶⁷³ (FIG. 12), (Zwick, 2004) single alanine substitution of either W⁶⁷² or F⁶⁷³ had a more drastic effect on 4E10 binding (<20% binding) than on CAP206-CH12 (30-40% binding) (FIG. 12). A critical residue for Z13e1 binding and neutralization, N671 and residues N-terminus to it (S⁶⁶⁸LW⁶⁷⁰), were not critical for CAP206-CH12 binding. Thus, the core epitope of CAP206-CH12 is slightly narrower and includes more C-terminus residues (W⁶⁷²FNI⁶⁷⁵) of gp41 MPER. However, in contrast to 4E10, CAP206-CH12 did not bind to either cardiolipin or PS containing liposomes and also failed to bind to MPER peptide liposomes complex (FIGS. 8E and 8F). Since CAP206-CH12 bound to the same peptide (MPER₆₅₆) in the absence of lipids, the lack of binding of CAP206-CH12 to MPER peptide liposomes reflects its inability to interact with lipids and extract membrane embedded critical residues.

Previously, 2F5 and 4E10 were shown to bind strongly with exceptionally slow off-rates to the trimeric gp41-inter, a protein that mimics the pre-hairpin intermediate state of gp41 (Frey et al, Proc. Natl. Acad. Sci. USA 105:3739-3744 (2008)). CAP206-CH12 bound to gp41-inter suggesting that CAP206-CH12 can recognize the MPER presented in the pre-hairpin conformation of gp41. However, when compared to 4E10 binding (Kd=1.6 nM; koff=1.5×10-5 s-1), CAP206-CH12 binding to gp41-inter was relatively weaker (Kd=23.3 nM) and displayed about 10-fold faster koff (kd=1.9×10-4 s-1). Taken together, the relatively weaker binding of CAP206-CH12 to gp41-inter and its lack of lipid binding could explain its lower neutralization potency when compared to those of 4E10.

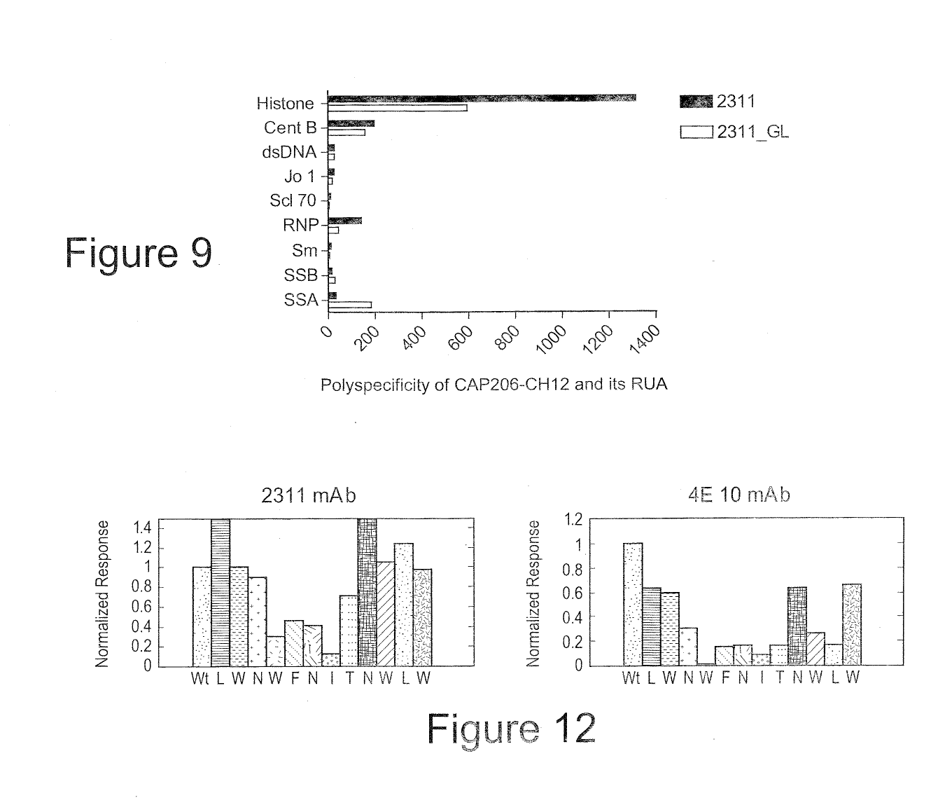

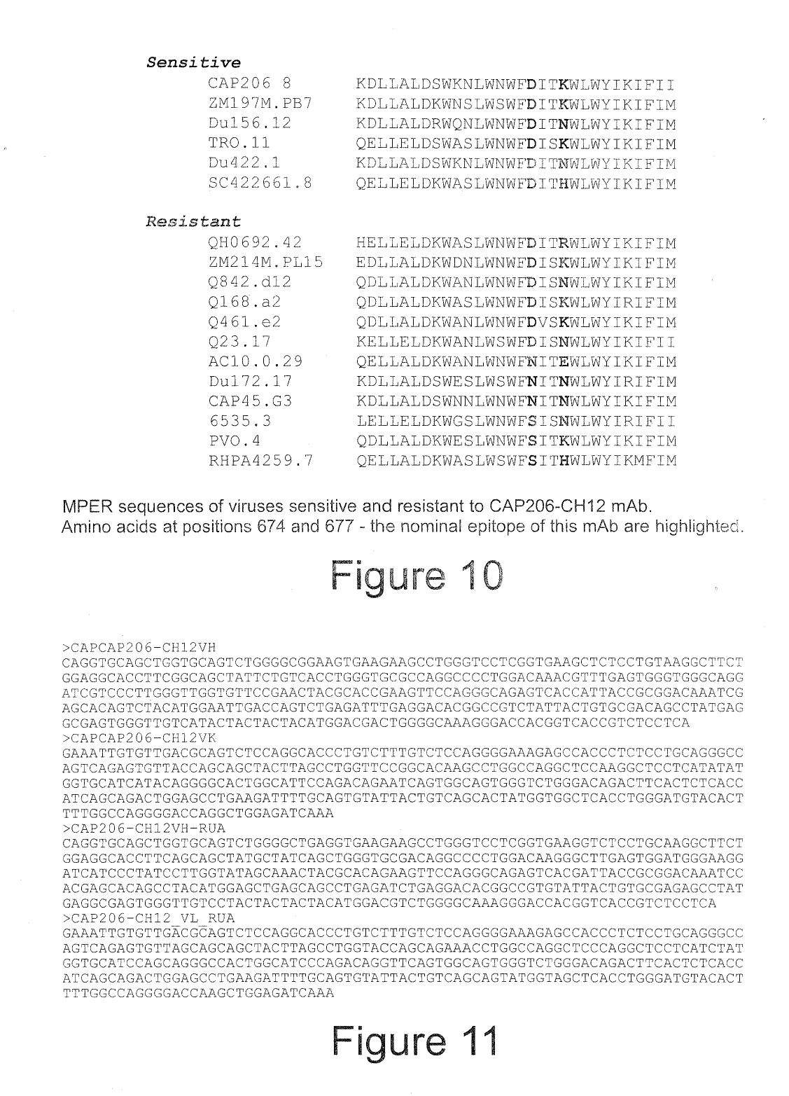

Like mAb 4E10, CAP206-CH12 was markedly polyreactive and reacted with histones, dsDNA and centromere autoantigens (FIG. 9). In Hep-2 cell fluorescence assay CAP206-CH12 was positive, and also reacted in luminex assay with normal gut flora whole cell extract (Table 17 below).

VH and VL Usage of CAP206-CH12:

Remarkably, mAb CAP206-CH12 used the same heavy and light chain families as the 4E10 mAb, namely VH1-69 and VK3-20. It also showed VH homology to another MPER mAb, Z13e1, with the presence of four H-CDR3 tyrosines and overall homology of 11/17 HCDR3 amino acids (Table 15). However, all 3 antibodies were genetically distinct as evidenced by their HCDR sequences. CAP206-CH12 has the shortest H-CDR3 (17 amino acids) and the longest L-CDR3 (11 amino acids) of the three antibodies.

TABLE 15A
VH and VL germ-line gene families and CDR sequences of CAP206
sorted B cells

| Antibody | V_{H} | | | | V_{L} | | | |
|---|---|---|---|---|---|---|---|---|
| ID | Family | CDR1 | CDR2 | CDR3 | Family | CDR1 | CDR2 | CDR3 |
| CAP206- | 1-69*04 | GGTFGSYS | IVPWVGVP | ATAYEASGLSYYYYMDD | 3-20*01 | QSVTSSY | GAS | QHYGGSPGMYT |
| H2311 | | | | | | | | |
| 4E10 | 1-69*10 | GGSFSTYA | VIPLLTIT | AREGTTGWGWLGKPIGAFAH | 3-20*01 | QSVGNNK | GAS | QQYGQSLST |
| Z13e1 | 4-59*03 | GGSMINYY | IIYGGTT | ARVAIGVSGFLNYYYYMDV | 3-11*01 | QSVGRN | DAS | QARLLLPQT |

TABLE 15B
Alignment of CAP206-CH12 with 4E10 and Z13.

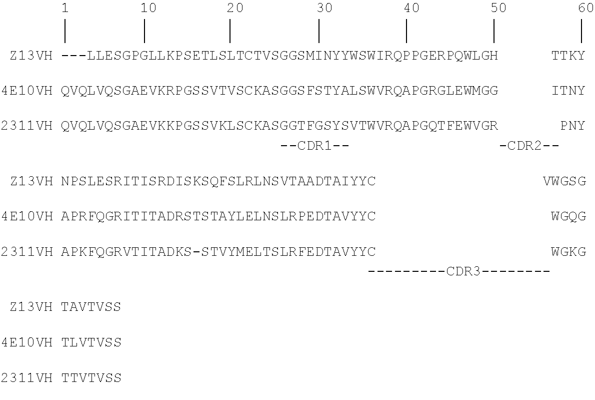

Neutralizing Activity of CAP206-CH12:

The functional activity of mAb CAP206-CH12 was tested in the TZM-bl pseudovirus neutralization assay using viruses against which the CAP206 plasma was active. Of the 6 viruses tested, 4 were shown to be sensitive to mAb CAP206-CH12 (Table 16A). This included the autologous virus as well as 2 subtype C and 1 subtype B virus. CAP206-CH12 when tested at 32 μg/ml did not neutralize 2 other viruses against which the plasma showed low levels of activity. Comparison of the IC₅₀ values suggested that CAP206-CH12 was similar in potency to the mAb Z13e1 and consistent with earlier data using polyclonal antibodies eluted from MPR.03 peptides (Gray et al, J. Virol. 83:8925-8937 (2009)). CAP206-CH12 was considerably less potent than mAb 4E10 (Gray et al, PLoS Med. 3:e255 (2006)). When tested against a large unselected panel of primary Tier 2 viruses of subtypes A, B and C, CAP206-CH12 neutralized only 2 of the 26 viruses (not shown).

TABLE 16A
CAP206-CH12 mAb neutralization of viruses sensitive to CAP206
plasma

| | | ID₅₀/IC₅₀ in TZM-bl cells¹ | | | | |
|---|---|---|---|---|---|---|
| | | CAP206 | CAP206- | | | |
| Virus | Subtype | plasma | H2311 | Z13e1 | 2F5 | 4E10 |
| CAP206.1.B5 | C | 6,143 | 5.9 | nd | >25 | 0.1 |
| ZM197M.PB7 | C | 256 | 13 | 30 | >25 | 1.1 |
| Du156.12 | C | 232 | 14.9 | 4.7 | >25 | 0.2 |
| TRO.11 | B | 212 | 17.5 | 13.3 | >25 | 0.3 |
| QHO692.42 | B | 125 | >32 | 46 | 1.81 | 6.5 |
| Du422.1 | C | 90 | >32 | nd | >25 | 0.3 |

¹Values are either the reciprocal plasma dilution (ID₅₀) or mAb concentration (IC₅₀, mg/ml) at which relative luminescence units (RLUs) were reduced 50% compared to virus control wells (no test sample).

Interestingly when a subset of these viruses was tested using TZM-bl cells in which the FcRγI receptor had been transfected, increased potency and breadth of CAP206-CH12 was observed as has been previously reported for mAb 4E10 (Table 16B) (Perez et al, J. Virol. 83:7397-7410 (2009)),. Thus, there was a 2-12 fold increase in sensitivity and two viruses (Du422.1 and SC422661.8) that were previously resistant were now sensitive to CAP206-CH12.

TABLE 16B
Enhancement of CAP206-CH12 neutralization
in TZM-bI expressing FcRγ1

| | | IC₅₀ in TZM-bI/FcγRI cells | | |
|---|---|---|---|---|
| Pseudovirus | Subtype | CAP206-H2311 | 2F5 | 4E10 |
| ZM197M.PB7 | C | 0.3 | 0.06 | <0.01 |
| SC422661.8 | B | 0.4 | <0.01 | <0.01 |
| Du156.12 | C | 0.6 | >25 | <0.01 |
| CAP206.1.B5 | C | 0.7 | >25 | <0.01 |
| Du422.1 | C | 2.7 | >25 | <0.01 |
| QH0692.42 | B | >32 | <0.01 | 0.11 |

¹Values are mAb concentration (IC₅₀, mg/ml) at which relative luminescence units (RLUs) were reduced 50% compared to virus control wells (no test sample).

Analysis of MPER sequences of CAP206-CH12 sensitive and resistance viruses showed that all had an aspartic acid at position 674 similar to the sequence present in the MPR.03 peptide (FIG. 9). The amino acid at position 677, the other site identified by alanine substitution mapping as important for CAP206-CH12 binding, was more variable with sensitive isolates tolerating K, N or H. QH0692.42 was sensitive to plasma antibodies but not to CAP206-CH12 and had the nominal D674 but had an asparagine at position 677 possibly accounting for its lack of CAP206-CH12 sensitivity. However, other isolates that had D674 and either K or N at 677 were also resistant suggesting that simply having the nominal epitope was not sufficient and other aspects such as exposure of the MPER are likely important in determining CAP206-CH12 sensitivity.

Characterization of Specificity and Reactivity of RUA of CAP206-CH12:

To understand the nature of the reactivity of the RUA, both CAP206-CH12 and CAP206-CH12_RUA were tested against a panel of HIV-1 and non HIV-1 antigens. The putative CAP206-CH12 germline, CAP206-CH12 RUA, bound to MPER.03 peptide but with a weaker binding Kd of 120 nM (FIG. 8), which was about 15-fold weaker than those of CAP206-CH12 mAb binding. CAP206-CH12 RUA also bound much weakly to gp41-inter with a Kd of 0.8 μM and koff (kd=3.5×10-3 s-1) which was about 20-fold faster than those of the mature CAP206-CH12 mAb.

CAP206-CH12 also reacted with HIV-1 g41, MOJO gp140 but also cross-reacted with non-HIV-1 antigens including hepatitis E2 protein and gut flora (Table 19 CAP206-CH12_RUA reacted with HIV-1 gp41 and also cross-reacted with hepatitis E2 protein and gut flora (Table 16).

This study, the power of epitope mapping of plasma antibody reactivity, rationale design of a memory B cell receptor ligand (bait), and single cell sorting with dual labeled ligands are demonstrated. Moreover, striking use of the same VH and VL families of the new MPER neutralizing mAb CAP206-CH12 as used by the prototype MPER mAb 4E10 is demonstrated. In addition, HCDR3 homology of CAP206-CH12 with broad neutralizing MPER mAb, Z13 is demonstrated.

The CAP206-CH12 mAb in the absence of target TZM-bl cells expressing FcRgamma1 receptors, did not have the same breadth as plasma antibodies, indicating that this type of antibody was responsible for a portion of the breadth observed in plasma. Nonetheless, the CAP206-CH12 mAb epitope directly overlapped the epitope of plasma antibodies indicating that it comprises a component of plasma neutralizing activity. While the CAP206-CH12 mAb was polyreactive for gut flora, histones and Hepatitis C E2 antigens, unlike 2F5 and 4E10 it did not bind lipids. Since both 2F5 and 4E10 require lipid reactivity for virion membrane binding in order to mediate neutralization, one hypothesis is that the neutralization potency of CAP206-CH12 may be limited by minimal lipid reactivity.

It was striking that CAP206-CH12 utilized the VH1-69 and VL κ3-20 utilized by the gp41 antibody 4E10. It has been reported that non-neutralizing human antibodies that bind to gp41 cluster II (N-terminal to the MPER) epitopes frequently use a VH1-69 Ig heavy chain (Xiao et al, BBRC (2009)). Other gp41 antibodies such as D5 that bind to the stalk of gp41 also utilize VH1-69 (Miller, PNAS (2005)). Another example of restricted usage of VH1-69 has recently been reported by the isolation of influenza broadly neutralizing antibodies to the stalk of hemagglutinin (Sui, Nat. Struct. Mol. Biol. (2009)). VH1-69 antibodies are hydrophobic and one hypothesis is that these antibodies are preferentially used for regions of virus envelopes that are in close proximity to viral membranes. Alternatively, Kipps and coworkers have reported that the percentage of the blood B cell repertoire that are VH1-69 antibodies are directly related to the VH1-69 copy number (Johnson et al, J. Immunol. 158:235 (1997)). Thus, both host and immunogen factors may give rise to preferential usage of VH1-69 in anti-viral responses.

Another striking finding was the similarity of the HCDR3 of CAP206-CH12 with that of the neutralizing MPER antibody, Z13e1 (Table 15B). While Z13e1 has VH 5-59, the sharing of aa motif LSY-YYYMD by the two antibodies likely represents convergent evolution of shaping of HCDR3s by similar antigenic regions.

TABLE 17
Comparison of the reactivity of mAb CAP206-CH12 with its RUA

| | | | MOJO | | MOJO | Anaerobic | | MOJO |
|---|---|---|---|---|---|---|---|---|
| Antibody ID | gp 41 | MPER.03 | gp140 | SP400 | gp120 | Gut Flora | HEP_E2 | gp140 |
| 2311 | 1978 | 23573 | 2909 | 1110 | — | 45 | 855 | 2909 |
| 2311-RUA | 2994 | — | — | 63 | — | 50 | 729 | — |

The epitope of Z13e1 spans residues S⁶⁶⁸LWNWFDITN⁶⁷⁷ (Nelson et al, J. Viral. 81:4033-3043 (2007)), while binding studies identified the epitope of CAP206-CH12 to WF(N/D)IT, which does not include residues N-terminus to W⁶⁷⁰. Both MPER mAbs have multiple CDR H3 Tyr residues. In the case of Z13e1, three of the Tyr residues positioned at the base of CDR H3 make contacts with the peptide (Pejchal et al, J. Virol. 83:8451-8462 (2009)) and thus CAP206-CH12 could potentially utilize the Tyr residues in a similar manner. It is notable that both 4E10 and Z13e1 have a flexible CDR H3 tip that bends away from the bound antigen (Cardoso et al., 2005; Pejchal et al, J. Virol. 83:8451-8462 (2009)). While 4E10 CDR H3 apex is involved in both lipid binding and neutralization (Alam et al., 2009), the flexibility of Z13e1 CDR H3 tip could allow it to engage the membrane—bound epitope (Pejchal et al, J. Virol. 83:8451-8462 (2009)). CAP206-CH12, which has a slightly shorter CDR H3, include some flexible residues adjacent to the Tyr motif but lacks hydrophobic residue W or F, which are present in both 4E10 and Z13e1 CDR H3 apex (4E10-GWGWLG; Z13e1-SGFLN). Since CAP206-CH12 did not bind to MPER peptide liposomes, in which MPER C-terminus hydrophobic residues are membrane immersed (Dennison et al., 2009), it is likely that CAP206-CH12 targets a different gp41 conformation, one in which the MPER is more solvent exposed. For MPER Nabs that bind to overlapping residues, differences in both orientation and conformation of gp41 recognized by 4E10 and Z13e1 have been described (Pejchal et al, J. Virol. 83:8451-8462 (2009); Cardoso et al., 2005). Based on the mapping and neutralization mutagenesis data, it is likely that CAP206-CH12 binds to a 4E10-favored W⁶⁷²/F⁶⁷³ accessible MPER conformation. However, unlike 4E10 and due to its lack of lipid reactivity, it might be not be able to access it until the core residues become fully exposed. Although it is possible that CAP206-CH12 might induce a rearrangement that exposes the core epitope, following the formation of an initial encounter complex. In spite of having overlapping epitopes, the MPER conformation recognized by CAP206-CH12, therefore, might be distinct from both Z13e1 and 4E10.

Finally, these studies show that epitope mapping of plasma antibodies followed by rational design of fluoresceinated Env subunits and successfully isolate antigen-reactive B cells. Scheid has previously used fluoresceinated whole Env for this purpose for isolation of Env-reactive B cells (Schied, Nature (2009)). The strategy used here combined an antigen specific probe with two color labeling to enhance the specificity of isolated antibodies.

The methods described above are expected to allow for the isolation of broadly neutralizing antibodies from many subjects with neutralizing antibody breadth. Study of the B cells and their reverted unmutated ancestors should prove useful in design of immunogens capable of activating naïve B cell receptors of naïve B cells that are capable of producing anti-HIV-1 antibodies with neutralizing breadth.

All documents and other information sources cited above are hereby incorporated in their entirety by reference.

### REFERENCES

1. Alam et al, J Virol, 2008; 82:115-25.
2. Babcook et al, Proc Natl Acad Sci USA. 1996; 93:7843-8.
3. Barreto et al, J Epidemiol Community Health. 2006; 60:192-5.
4. Binley et al, J. Virol. 2000; 74:627-43.
5. Boshart et al, Cell. 1985; 41:521-30.
6. Brezinschek et al, J Immunol, 1995; 155:190-202.
7. Burstein et al, Biochemistry. 1978; 17:2392-2400.
8. Connelly et al, Genes Dev. 1988; 2:440-52.
9. Croft et al, J Virol Methods. 2008; 153:203-13.
10. Di Noia et al, Annu Rev Biochem. 2007; 76:1-22.
11. Diaz et al, Curr Opin Immunol. 2002; 14:235-40.
12. Gimmi et al, Nucleic Acids Res. 1989; 17:6983-98.
13. Honjo et al, Annu Rev Biochem. 1985; 54:803-30.
14, Kim et al, Cell. 1981; 27:573-81.
15. Kirchherr et al, J Virol Methods. 2007; 143:104-11.
16. Koelsch et al, J Clin Invest. 2007; 117:1558-65.
17, Kunert et al, AIDS Res Hum Retroviruses. 1998; 14:1115-28.
18. Kuppers et al, Embo J. 1993; 12:4955-67.
19. Liao et al, Virology. 2006; 353:268-282.
20. Market et al, PLoS Biol. 2003; 1:E16.
21. McLean et al, Mol. Immunol. 2000; 37:837-15.
22. Montefiori D C, Curr Protoc Immunol. 2005;Chapter 12(Unit 1211)
23. Norderhaug et al, J Immunol Methods. 1997; 204:77-87.
24. Ofek t al, J. Virol. 2004; 78:10724-37.
25. Owens et al, J. Immunol. 2003; 171:2725-33.
26. Persic et al, Gene. 1997; 187:9-18,
27. Robinson W H, Curr Opin Chem. Biol. 2006; 10:67-72.
28. Strausberg et al, Proc Natl Acad Sci USA. 2002; 99:16899-903.
29. Tiller et al, J Immunol Methods. 2008; 329:112-24.
30. Tonegawa S, Nature. 1983; 302:575-81.
31. Traggiai et al, Nat. Med. 2004; 10:871-5.
32. Volkheimer et al, Blood. 2007; 109:1559-67.
33. Wang et al, J Immunol Methods. 2000; 244:217-25.
34. Wardemann et al, Science. 2003; 301:1374-7.
35. Wrammert et al, Nature. 2008; 453:667-71.
36. Xiao et al, Molecular Biotechnology. 2007; 35
37. Yu et al, J Immunol Methods. 2008; 336:142-51.

What is claimed is:

1. An isolated antibody, or antigen binding fragment thereof, comprising:
i) a heavy chain variable region (HCVR), the complementarity determining regions (CDRs) of said HCVR comprising the amino acid sequences GGTFGSYS, IVPWVGVP and TAYEASGLSYYYYMDD, and
ii) a light chain variable region (LCVR), the CDRs of said LCVR comprising the amino acid sequences QSVTSSY, GAS and QHYGGSPGMYT.

2. The antibody according to claim 1 wherein said antibody is a monoclonal antibody.

3. The antibody according to claim 1 wherein said antibody is a human or humanized antibody.

4. A composition comprising the antibody according to claim 1, or said fragment thereof, and a carrier.

5. An isolated nucleic acid encoding the antibody according to claim 1, or said fragment thereof.

6. A vector comprising the nucleic acid according to claim 5, wherein said nucleic acid is present in said vector in operable linkage with a promoter.

7. A host cell comprising the vector according to claim 6.

8. A composition comprising the vector according to claim 6 and a carrier.

9. A method of inhibiting HIV-1 infection in a patient comprising administering to said patient said antibody according to claim 1, or said fragment thereof, in an amount sufficient to inhibit said infection.

10. The method according to claim 9 wherein said antibody is administered to a mucosal surface of said patient.

11. A linear Ig gene expression cassette comprising, in the 5′ to 3′ direction, a promoter operably linked to a Ig leader sequence, a V_{H} or V_{L} gene sequence, a constant region sequence of IgG1 heavy-chain or light-chain and a poly A tail.

12. The gene expression cassette according to claim 11 wherein said V_{H} or V_{L} gene is a human or mouse gene.

13. A method of producing a monoclonal antibody comprising introducing into a host cell the linear expression cassette according to claim 11 under conditions such that said monoclonal antibody is expressed.

14. A host cell comprising the linear gene expression cassette according to claim 11.


SEQUENCE LISTING (201 sequences)

SEQ ID NO: 1 - protein, 26 aa, Human immunodeficiency virus 1
NEQELLELDKWASLWNWFDITNWLWY

SEQ ID NO: 2 - DNA, 467 bp, Artificial Sequence
  Description of Artificial Sequence Synthetic polynucleotide
caggtgcagctggtgcagtctggggcggaagtgaagaagcctgggtcctcggtgaagctctcctgtaaggcttctggaggcaccttcggcagctattctgtcacctgggtgcgccaggcccctggacaaacgtttgagtgggtgggcaggatcgtcccttgggttggtgttccgaactacgcaccgaagttccagggcagagtcaccattaccgcggacaaatcgagcacagtctacatggaattgaccagtctgagatttgaggacacggccgtctattactgtgcgacagcctatgaggcgagtgggttgtcatactactactacatggacgactggggcaaagggaccacggtcaccgtctcctcagcctccaccaagggcccatcggtcttccccctggcaccctcctccaagagcacctctggggcaaaaaaggggccaaagcgggggaaacccccaggagc

SEQ ID NO: 3 - protein, 123 aa, Artificial Sequence
  Description of Artificial Sequence Synthetic polypeptide
QVQLVQSGAEVKKPGSSVKLSCKASGGTFGSYSVTWVRQAPGQTFEWVGRIVPWVGVPNYAPKFQGRVTITADKSSTVYMELTSLRFEDTAVYYCATAYEASGLSYYYYMDDWGKGTTVTVSS

SEQ ID NO: 4 - DNA, 330 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttaccagcagctacttagcctggttccggcacaagcctggccaggctccaaggctcctcatatatggtgcatcatacaggggcactggcattccagacagaatcagtggcagtgggtctgggacagacttcactctcaccatcagcagactggagcctgaagattttgcagtgtattactgtcagcactatggtggctcacctgggatgtacacttttggccaggggaccaggctggagatcaaa

SEQ ID NO: 5 - protein, 110 aa, Artificial Sequence
  Description of Artificial Sequence Synthetic polypeptide
EIVLTQSPGTLSLSPGERATLSCRASQSVTSSYLAWFRHKPGQAPRLLIYGASYRGTGIPDRISGSGSGTDFTLTISRLEPEDFAVYYCQHYGGSPGMYTFGQGTRLEIK

SEQ ID NO: 6 - protein, 34 aa, Artificial Sequence
  Description of Artificial Sequence Synthetic peptide
KKKNEQELLELDKWASLWNWFDITNWLWYIRKKK

SEQ ID NO: 7 - protein, 33 aa, Artificial Sequence
  Description of Artificial Sequence Synthetic polypeptide
TRPGNNTRKSIRIGPGQTFFATGDIIGDIREAH

SEQ ID NO: 8 - protein, 34 aa, Artificial Sequence
  Description of Artificial Sequence Synthetic polypeptide
NMYELQKLNSWDVFGNWFDLASWVKYIQYGVYIV

SEQ ID NO: 9 - protein, 34 aa, Artificial Sequence
  Description of Artificial Sequence Synthetic polypeptide
NMYELLALDKWASLWNWFDITKWLWYIKYGVYIV

SEQ ID NO: 10 - protein, 34 aa, Artificial Sequence
  Description of Artificial Sequence Synthetic polypeptide
NMYELLALDSWKNLWNWFDITKWLWYIKYGVYIV

SEQ ID NO: 11 - protein, 34 aa, Artificial Sequence
  Description of Artificial Sequence Synthetic polypeptide
NMYELLALDSWKNLWNWLDITKWLWYIKYGVYIV

SEQ ID NO: 12 - protein, 34 aa, Artificial Sequence
  Description of Artificial Sequence Synthetic polypeptide
NMYELLALDKWASLWNWFDLASWVKYIQYGVYIV

SEQ ID NO: 13 - protein, 34 aa, Homo sapiens
NMYELQALDKWAVFGNWFDLASWVKYIQYGVYIV

SEQ ID NO: 14 - protein, 34 aa, Homo sapiens
NMYELQKLNSWDVFGNWFDITSWIKYIQYGVYIV

SEQ ID NO: 15 - protein, 34 aa, Artificial Sequence
  Description of Artificial Sequence Synthetic polypeptide
NMYELQKLNSWDVFGNWFDITKWLWYIKYGVYIV

SEQ ID NO: 16 - protein, 34 aa, Artificial Sequence
  Description of Artificial Sequence Synthetic polypeptide
NMYELQKLNSWDVFWNWFDITKWLWYIKYGVYIV

SEQ ID NO: 17 - protein, 34 aa, Artificial Sequence
  Description of Artificial Sequence Synthetic polypeptide
NMYELQKLNSWDSLWNWFDITKWLWYIKYGVYIV

SEQ ID NO: 18 - protein, 34 aa, Artificial Sequence; identical to SEQ ID NO: 6
  Description of Artificial Sequence Synthetic polypeptide

SEQ ID NO: 19 - DNA, 20 bp, Artificial Sequence
  Description of Artificial Sequence Synthetic primer
atggagtcgggaaggaagtc

SEQ ID NO: 20 - DNA, 19 bp, Artificial Sequence
  Description of Artificial Sequence Synthetic primer
tcacggacgttgggtggta

SEQ ID NO: 21 - DNA, 19 bp, Artificial Sequence
  Description of Artificial Sequence Synthetic primer
tcacggaggtggcattgga

SEQ ID NO: 22 - DNA, 19 bp, Artificial Sequence
  Description of Artificial Sequence Synthetic primer
caggcgatgaccacgttcc

SEQ ID NO: 23 - DNA, 19 bp, Artificial Sequence
  Description of Artificial Sequence Synthetic primer
catgcgacgaccacgttcc

SEQ ID NO: 24 - DNA, 20 bp, Artificial Sequence
  Description of Artificial Sequence Synthetic primer
aggtgtgcacgccgctggtc

SEQ ID NO: 25 - DNA, 20 bp, Artificial Sequence
  Description of Artificial Sequence Synthetic primer
gcaggcacacaacagaggca

SEQ ID NO: 26 - DNA, 17 bp, Artificial Sequence
  Description of Artificial Sequence Synthetic primer
aggccactgtcacagct

SEQ ID NO: 27 - DNA, 20 bp, Artificial Sequence
  Description of Artificial Sequence Synthetic primer
ccatggactggacctggagg

SEQ ID NO: 28 - DNA, 19 bp, Artificial Sequence
  Description of Artificial Sequence Synthetic primer
atggacatactttgttcca

SEQ ID NO: 29 - DNA, 20 bp, Artificial Sequence
  Description of Artificial Sequence Synthetic primer
ccatggagtttgggctgagc

SEQ ID NO: 30 - DNA, 20 bp, Artificial Sequence
  Description of Artificial Sequence Synthetic primer
atgaaacacctgtggttctt

SEQ ID NO: 31 - DNA, 20 bp, Artificial Sequence
  Description of Artificial Sequence Synthetic primer
atggggtcaaccgccatcct

SEQ ID NO: 32 - DNA, 20 bp, Artificial Sequence
  Description of Artificial Sequence Synthetic primer
atgtctgtctccttcctcat

SEQ ID NO: 33 - DNA, 17 bp, Artificial Sequence
  Description of Artificial Sequence Synthetic primer
gctcagctcctggggct

SEQ ID NO: 34 - DNA, 17 bp, Artificial Sequence
  Description of Artificial Sequence Synthetic primer
ggaarccccagcdcagc

SEQ ID NO: 35 - DNA, 19 bp, Artificial Sequence
  Description of Artificial Sequence Synthetic primer
ctsttsctytggatctctg

SEQ ID NO: 36 - DNA, 17 bp, Artificial Sequence
  Description of Artificial Sequence Synthetic primer
ctsctgctctgggytcc

SEQ ID NO: 37 - DNA, 20 bp, Artificial Sequence
  Description of Artificial Sequence Synthetic primer
cgaygaccacgttcccatct

SEQ ID NO: 38 - DNA, 24 bp, Artificial Sequence
  Description of Artificial Sequence Synthetic primer
ctgttatcctttgggtgtctgcac

SEQ ID NO: 39 - DNA, 20 bp, Artificial Sequence
  Description of Artificial Sequence Synthetic primer
cgcctgagttccacgacacc

SEQ ID NO: 40 - DNA, 20 bp, Artificial Sequence
  Description of Artificial Sequence Synthetic primer
ccgacggggaattctcacag

SEQ ID NO: 41 - DNA, 20 bp, Artificial Sequence
  Description of Artificial Sequence Synthetic primer
gaggcagttccagatttcaa

SEQ ID NO: 42 - DNA, 17 bp, Artificial Sequence
  Description of Artificial Sequence Synthetic primer
cctgggcccagtctgtg

SEQ ID NO: 43 - DNA, 18 bp, Artificial Sequence
  Description of Artificial Sequence Synthetic primer
ctcctcasyctcctcact

SEQ ID NO: 44 - DNA, 18 bp, Artificial Sequence
  Description of Artificial Sequence Synthetic primer
ggcctcctatgwgctgac

SEQ ID NO: 45 - DNA, 23 bp, Artificial Sequence
  Description of Artificial Sequence Synthetic primer
gttctgtggtttcttctgagctg

SEQ ID NO: 46 - DNA, 18 bp, Artificial Sequence
  Description of Artificial Sequence Synthetic primer
acagggtctctctcccag

SEQ ID NO: 47 - DNA, 19 bp, Artificial Sequence
  Description of Artificial Sequence Synthetic primer
acaggtctctgtgctctgc

SEQ ID NO: 48 - DNA, 17 bp, Artificial Sequence
  Description of Artificial Sequence Synthetic primer
ccctctcscagsctgtg

SEQ ID NO: 49 - DNA, 19 bp, Artificial Sequence
  Description of Artificial Sequence Synthetic primer
tcttgggccaattttatgc

SEQ ID NO: 50 - DNA, 19 bp, Artificial Sequence
  Description of Artificial Sequence Synthetic primer
attcycagrctgtggtgac

SEQ ID NO: 51 - DNA, 17 bp, Artificial Sequence
  Description of Artificial Sequence Synthetic primer
cagtggtccaggcaggg

SEQ ID NO: 52 - DNA, 17 bp, Artificial Sequence
  Description of Artificial Sequence Synthetic primer
aggccactgtcacagct

SEQ ID NO: 53 - DNA, 25 bp, Artificial Sequence
  Description of Artificial Sequence Synthetic primer
ctgggttccaggttccactggtgac

SEQ ID NO: 54 - DNA, 24 bp, Artificial Sequence
  Description of Artificial Sequence Synthetic primer
caggtgcagctggtrcagtctggg

SEQ ID NO: 55 - DNA, 25 bp, Artificial Sequence
  Description of Artificial Sequence Synthetic primer
ctgggttccaggttccactggtgac

SEQ ID NO: 56 - DNA, 26 bp, Artificial Sequence
  Description of Artificial Sequence Synthetic primer
cagrgcaccttgarggagtctggtcc

SEQ ID NO: 57 - DNA, 25 bp, Artificial Sequence
  Description of Artificial Sequence Synthetic primer
ctgggttccaggttccactggtgac

SEQ ID NO: 58 - DNA, 24 bp, Artificial Sequence
  Description of Artificial Sequence Synthetic primer
gaggtkcagctggtggagtctggg

SEQ ID NO: 59 - DNA, 25 bp, Artificial Sequence
  Description of Artificial Sequence Synthetic primer
ctgggttccaggttccactggtgac

SEQ ID NO: 60 - DNA, 22 bp, Artificial Sequence
  Description of Artificial Sequence Synthetic primer
caggtgcagctgcaggagtcgg

SEQ ID NO: 61 - DNA, 25 bp, Artificial Sequence
  Description of Artificial Sequence Synthetic primer
ctgggttccaggttccactggtgac

SEQ ID NO: 62 - DNA, 25 bp, Artificial Sequence
  Description of Artificial Sequence Synthetic primer
gargtgcagctggtgcagtctggag

SEQ ID NO: 63 - DNA, 25 bp, Artificial Sequence
  Description of Artificial Sequence Synthetic primer
ctgggttccaggttccactggtgac

SEQ ID NO: 64 - DNA, 26 bp, Artificial Sequence
  Description of Artificial Sequence Synthetic primer
caggtacagctgcagcagtcaggtcc

SEQ ID NO: 65 - DNA, 17 bp, Artificial Sequence
  Description of Artificial Sequence Synthetic primer
gctgtgcccccagaggt

SEQ ID NO: 66 - DNA, 21 bp, Artificial Sequence
  Description of Artificial Sequence Synthetic primer
gctggtgctgcagaggctcag

SEQ ID NO: 67 - DNA, 17 bp, Artificial Sequence
  Description of Artificial Sequence Synthetic primer
gctgtgcccccagaggt

SEQ ID NO: 68 - DNA, 21 bp, Artificial Sequence
  Description of Artificial Sequence Synthetic primer
gctggtgctgtcgaggctcag

SEQ ID NO: 69 - DNA, 17 bp, Artificial Sequence
  Description of Artificial Sequence Synthetic primer
gctgtgcccccagaggt

SEQ ID NO: 70 - DNA, 23 bp, Artificial Sequence
  Description of Artificial Sequence Synthetic primer
gtgtctgcaccctgatatgatgg

SEQ ID NO: 71 - DNA, 17 bp, Artificial Sequence
  Description of Artificial Sequence Synthetic primer
gctgtgcccccagaggt

SEQ ID NO: 72 - DNA, 17 bp, Artificial Sequence
  Description of Artificial Sequence Synthetic primer
gctgtgcccccagaggt

SEQ ID NO: 73 - DNA, 22 bp, Artificial Sequence
  Description of Artificial Sequence Synthetic primer
ggaattctcacaggagacgagg

SEQ ID NO: 74 - DNA, 25 bp, Artificial Sequence
  Description of Artificial Sequence Synthetic primer
ctgggttccaggttccactggtgac

SEQ ID NO: 75 - DNA, 22 bp, Artificial Sequence
  Description of Artificial Sequence Synthetic primer
gacatccagwtgacccagtctc

SEQ ID NO: 76 - DNA, 25 bp, Artificial Sequence
  Description of Artificial Sequence Synthetic primer
ctgggttccaggttccactggtgac

SEQ ID NO: 77 - DNA, 25 bp, Artificial Sequence
  Description of Artificial Sequence Synthetic primer
gatattgtgatgacccagwctccac

SEQ ID NO: 78 - DNA, 25 bp, Artificial Sequence
  Description of Artificial Sequence Synthetic primer
ctgggttccaggttccactggtgac

SEQ ID NO: 79 - DNA, 24 bp, Artificial Sequence
  Description of Artificial Sequence Synthetic primer
gaaattgtgttgacrcagtctcca

SEQ ID NO: 80 - DNA, 25 bp, Artificial Sequence
  Description of Artificial Sequence Synthetic primer
ctgggttccaggttccactggtgac

SEQ ID NO: 81 - DNA, 22 bp, Artificial Sequence
  Description of Artificial Sequence Synthetic primer
gacatcgtgatgacccagtctc

SEQ ID NO: 82 - DNA, 25 bp, Artificial Sequence
  Description of Artificial Sequence Synthetic primer
ctgggttccaggttccactggtgac

SEQ ID NO: 83 - DNA, 22 bp, Artificial Sequence
  Description of Artificial Sequence Synthetic primer
gaaacgacactcacgcagtctc

SEQ ID NO: 84 - DNA, 25 bp, Artificial Sequence
  Description of Artificial Sequence Synthetic primer
ctgggttccaggttccactggtgac

SEQ ID NO: 85 - DNA, 24 bp, Artificial Sequence
  Description of Artificial Sequence Synthetic primer
gaaattgtgctgacwcagtctcca

SEQ ID NO: 86 - DNA, 25 bp, Artificial Sequence
  Description of Artificial Sequence Synthetic primer
ctgggttccaggttccactggtgac

SEQ ID NO: 87 - DNA, 21 bp, Artificial Sequence
  Description of Artificial Sequence Synthetic primer
gacattgtgctgacccagtct

SEQ ID NO: 88 - DNA, 21 bp, Artificial Sequence
  Description of Artificial Sequence Synthetic primer
gggaagatgaagacagatggt

SEQ ID NO: 89 - DNA, 25 bp, Artificial Sequence
  Description of Artificial Sequence Synthetic primer
ctgggttccaggttccactggtgac

SEQ ID NO: 90 - DNA, 20 bp, Artificial Sequence
  Description of Artificial Sequence Synthetic primer
cagtctgtgytgackcagcc

SEQ ID NO: 91 - DNA, 25 bp, Artificial Sequence
  Description of Artificial Sequence Synthetic primer
ctgggttccaggttccactggtgac

SEQ ID NO: 92 - DNA, 20 bp, Artificial Sequence
  Description of Artificial Sequence Synthetic primer
cagtctgccctgactcagcc

SEQ ID NO: 93 - DNA, 25 bp, Artificial Sequence
  Description of Artificial Sequence Synthetic primer
ctgggttccaggttccactggtgac

SEQ ID NO: 94 - DNA, 22 bp, Artificial Sequence
  Description of Artificial Sequence Synthetic primer
tcytatgagctgacwcagccac

SEQ ID NO: 95 - DNA, 25 bp, Artificial Sequence
  Description of Artificial Sequence Synthetic primer
ctgggttccaggttccactggtgac

SEQ ID NO: 96 - DNA, 23 bp, Artificial Sequence
  Description of Artificial Sequence Synthetic primer
tcttctgagctgactcaggaccc

SEQ ID NO: 97 - DNA, 25 bp, Artificial Sequence
  Description of Artificial Sequence Synthetic primer
ctgggttccaggttccactggtgac

SEQ ID NO: 98 - DNA, 20 bp, Artificial Sequence
  Description of Artificial Sequence Synthetic primer
cagcytgtgctgactcaatc

SEQ ID NO: 99 - DNA, 25 bp, Artificial Sequence
  Description of Artificial Sequence Synthetic primer
ctgggttccaggttccactggtgac

SEQ ID NO: 100 - DNA, 19 bp, Artificial Sequence
  Description of Artificial Sequence Synthetic primer
ctgcctgtgctgactcagc

SEQ ID NO: 101 - DNA, 25 bp, Artificial Sequence
  Description of Artificial Sequence Synthetic primer
ctgggttccaggttccactggtgac

SEQ ID NO: 102 - DNA, 20 bp, Artificial Sequence
  Description of Artificial Sequence Synthetic primer
cagsctgtgctgactcagcc

SEQ ID NO: 103 - DNA, 25 bp, Artificial Sequence
  Description of Artificial Sequence Synthetic primer
ctgggttccaggttccactggtgac

SEQ ID NO: 104 - DNA, 25 bp, Artificial Sequence
  Description of Artificial Sequence Synthetic primer
aattttatgctgactcagccccact

SEQ ID NO: 105 - DNA, 25 bp, Artificial Sequence
  Description of Artificial Sequence Synthetic primer
ctgggttccaggttccactggtgac

SEQ ID NO: 106 - DNA, 21 bp, Artificial Sequence
  Description of Artificial Sequence Synthetic primer
cagrctgtggtgacycaggag

SEQ ID NO: 107 - DNA, 25 bp, Artificial Sequence
  Description of Artificial Sequence Synthetic primer
ctgggttccaggttccactggtgac

SEQ ID NO: 108 - DNA, 18 bp, Artificial Sequence
  Description of Artificial Sequence Synthetic primer
caggcagggcwgactcag

SEQ ID NO: 109 - DNA, 19 bp, Artificial Sequence
  Description of Artificial Sequence Synthetic primer
gggygggaacagagtgacc

SEQ ID NO: 110 - protein, 21 aa, Homo sapiens
METDTLLLWVLLLWVPGSTGD

SEQ ID NO: 111 - DNA, 26 bp, Artificial Sequence
  Description of Artificial Sequence Synthetic primer
tctgggttccaggttccactggtgac

SEQ ID NO: 112 - DNA, 26 bp, Artificial Sequence
  Description of Artificial Sequence Synthetic primer
tctgggttccaggttccactggtgac

SEQ ID NO: 113 - DNA, 26 bp, Artificial Sequence
  Description of Artificial Sequence Synthetic primer
tctgggttccaggttccactggtgac

SEQ ID NO: 114 - DNA, 31 bp, Artificial Sequence
  Description of Artificial Sequence Synthetic primer
agtaatcaattacggggtcattagttcatag

SEQ ID NO: 115 - DNA, 18 bp, Artificial Sequence
  Description of Artificial Sequence Synthetic primer
cacctctgggggcacagc

SEQ ID NO: 116 - DNA, 30 bp, Artificial Sequence
  Description of Artificial Sequence Synthetic primer
cgaactgtggctgcaccatctgtcttcatc

SEQ ID NO: 117 - DNA, 28 bp, Artificial Sequence
  Description of Artificial Sequence Synthetic primer
tgccccctcggtcactctgttcccgccc

SEQ ID NO: 118 - DNA, 18 bp, Artificial Sequence
  Description of Artificial Sequence Synthetic primer
gctgtgcccccagaggtg

SEQ ID NO: 119 - DNA, 24 bp, Artificial Sequence
  Description of Artificial Sequence Synthetic primer
gacagatggtgcagccacagttcg

SEQ ID NO: 120 - DNA, 21 bp, Artificial Sequence
  Description of Artificial Sequence Synthetic primer
cagagtgaccgagggggcagc

SEQ ID NO: 121 - DNA, 25 bp, Artificial Sequence
  Description of Artificial Sequence Synthetic primer
gtcaccagtggaacctggaacccag

SEQ ID NO: 122 - DNA, 23 bp, Artificial Sequence
  Description of Artificial Sequence Synthetic primer
tccccagcatgcctgctattgtc

SEQ ID NO: 123 - DNA, 23 bp, Artificial Sequence
  Description of Artificial Sequence Synthetic primer
tccccagcatgcctgctattgtc

SEQ ID NO: 124 - DNA, 23 bp, Artificial Sequence
  Description of Artificial Sequence Synthetic primer
tccccagcatgcctgctattgtc

SEQ ID NO: 125 - protein, 20 aa, Human immunodeficiency virus 1
QQEKNEQELLELDKWASLWN

SEQ ID NO: 126 - protein, 44 aa, Human immunodeficiency virus 1
RVLAVERYLRDQQLLGIWGCSGKLICTTAVPWNASWSNKSLNKI

SEQ ID NO: 127 - protein, 20 aa, Artificial Sequence
  Description of Artificial Sequence Synthetic peptide
NKEQDQAEESLQLWEKLNWL

SEQ ID NO: 128 - protein, 25 aa, Homo sapiens
NEQELLELDKWASLWNWFNITNWLW

SEQ ID NO: 129 - protein, 8 aa, Artificial Sequence
  Description of Artificial Sequence Synthetic peptide
GGTFGSYS

SEQ ID NO: 130 - protein, 8 aa, Homo sapiens
GGSFSTYA

SEQ ID NO: 131 - protein, 8 aa, Artificial Sequence
  Description of Artificial Sequence Synthetic peptide
GGSMINYY

SEQ ID NO: 132 - protein, 8 aa, Artificial Sequence
  Description of Artificial Sequence Synthetic peptide
IVPWVGVP

SEQ ID NO: 133 - protein, 8 aa, Homo sapiens
VIPLLTIT

SEQ ID NO: 134 - protein, 7 aa, Artificial Sequence
  Description of Artificial Sequence Synthetic peptide
IIYGGTT

SEQ ID NO: 135 - protein, 17 aa, Artificial Sequence
  Description of Artificial Sequence Synthetic peptide
ATAYEASGLSYYYYMDD

SEQ ID NO: 136 - protein, 20 aa, Homo sapiens
AREGTTGWGWLGKPIGAFAH

SEQ ID NO: 137 - protein, 19 aa, Artificial Sequence
  Description of Artificial Sequence Synthetic peptide
ARVAIGVSGFLNYYYYMDV

SEQ ID NO: 138 - protein, 7 aa, Artificial Sequence
  Description of Artificial Sequence Synthetic peptide
QSVTSSY

SEQ ID NO: 139 - protein, 7 aa, Homo sapiens
QSVGNNK

SEQ ID NO: 140 - protein, 6 aa, Artificial Sequence
  Description of Artificial Sequence Synthetic peptide
QSVGRN

SEQ ID NO: 141 - protein, 11 aa, Artificial Sequence
  Description of Artificial Sequence Synthetic peptide
QHYGGSPGMYT

SEQ ID NO: 142 - protein, 9 aa, Homo sapiens
QQYGQSLST

SEQ ID NO: 143 - protein, 9 aa, Artificial Sequence
  Description of Artificial Sequence Synthetic peptide
QARLLLPQT

SEQ ID NO: 144 - protein, 122 aa, Artificial Sequence
  Description of Artificial Sequence Synthetic polypeptide
LLESGPGLLKPSETLSLTCTVSGGSMINYYWSWIRQPPGERPQWLGHIIYGGTTKYNPSLESRITISRDISKSQFSLRLNSVTAADTAIYYCARVAIGVSGFLNYYYYMDVWGSGTAVTVSS

SEQ ID NO: 145 - protein, 127 aa, Homo sapiens
QVQLVQSGAEVKRPGSSVTVSCKASGGSFSTYALSWVRQAPGRGLEWMGGVIPLLTITNYAPRFQGRITITADRSTSTAYLELNSLRPEDTAVYYCAREGTTGWGWLGKPIGAFAHWGQGTLVTVSS

SEQ ID NO: 146 - protein, 8 aa, Artificial Sequence
  Description of Artificial Sequence Synthetic peptide
LSYYYYMD

SEQ ID NO: 147 - protein, 10 aa, Artificial Sequence
  Description of Artificial Sequence Synthetic peptide
SLWNWFDITN

SEQ ID NO: 148 - protein, 5 aa, Artificial Sequence
  Description of Artificial Sequence Synthetic peptide
WFXIT

SEQ ID NO: 149 - protein, 6 aa, Homo sapiens
GWGWLG

SEQ ID NO: 150 - protein, 5 aa, Artificial Sequence
  Description of Artificial Sequence Synthetic peptide
SGFLN

SEQ ID NO: 151 - protein, 16 aa, Artificial Sequence
  Description of Artificial Sequence Synthetic peptide
TAYEASGLSYYYYMDD

SEQ ID NO: 152 - protein, 34 aa, Artificial Sequence; identical to SEQ ID NO: 10
  Description of Artificial Sequence Synthetic polypeptide

SEQ ID NO: 153 - protein, 34 aa, Artificial Sequence; identical to SEQ ID NO: 16
  Description of Artificial Sequence Synthetic polypeptide

SEQ ID NO: 154 - protein, 34 aa, Artificial Sequence; identical to SEQ ID NO: 15
  Description of Artificial Sequence Synthetic polypeptide

SEQ ID NO: 155 - protein, 34 aa, Artificial Sequence; identical to SEQ ID NO: 14
  Description of Artificial Sequence Synthetic polypeptide

SEQ ID NO: 156 - protein, 34 aa, Artificial Sequence; identical to SEQ ID NO: 8
  Description of Artificial Sequence Synthetic polypeptide

SEQ ID NO: 157 - protein, 35 aa, Artificial Sequence
  Description of Artificial Sequence Synthetic polypeptide
QQEKNEKDLLALDSWKNLWNWFDITKWLWYIKIFI

SEQ ID NO: 158 - protein, 35 aa, Artificial Sequence
  Description of Artificial Sequence Synthetic polypeptide
QQEKNEKDLLALDSWKNLWNWFDITKWLWYIRIFI

SEQ ID NO: 159 - protein, 35 aa, Artificial Sequence
  Description of Artificial Sequence Synthetic polypeptide
QQEKNEKDLPALDSWKNLWNWFDITKWLWYIRIFI

SEQ ID NO: 160 - protein, 35 aa, Artificial Sequence
  Description of Artificial Sequence Synthetic polypeptide
QQEKNEKDLLALDSWKNLWNWFDITNWLWYIQIFI

SEQ ID NO: 161 - protein, 35 aa, Artificial Sequence
  Description of Artificial Sequence Synthetic polypeptide
QQEKNEKDLLALDSWKNLWTWFDITNWLWYIKIFI

SEQ ID NO: 162 - protein, 35 aa, Artificial Sequence
  Description of Artificial Sequence Synthetic polypeptide
QQEKNEKDLLALDSWKNLWNWFDITNWLWYIKIFI

SEQ ID NO: 163 - protein, 35 aa, Artificial Sequence
  Description of Artificial Sequence Synthetic polypeptide
QQEKNEKDLLALDSWKNLWNWFDITNWLRYIQIFI

SEQ ID NO: 164 - protein, 35 aa, Artificial Sequence
  Description of Artificial Sequence Synthetic polypeptide
QQEKNEKDLLALDSWKNLWTWFDITNWLWYIRIFI

SEQ ID NO: 165 - protein, 35 aa, Artificial Sequence
  Description of Artificial Sequence Synthetic polypeptide
QQEKNEKDLLALDSWKNLWNWFNITNWLWYIKIFI

SEQ ID NO: 166 - protein, 35 aa, Artificial Sequence
  Description of Artificial Sequence Synthetic polypeptide
QQEKNEKDLLALDSWKNLWNWFDISNWLRYIQIFI

SEQ ID NO: 167 - protein, 35 aa, Artificial Sequence
  Description of Artificial Sequence Synthetic polypeptide
QQEKNEKDLLALDSWKNLWTWFNISNWLWYIRIFI

SEQ ID NO: 168 - protein, 35 aa, Artificial Sequence
  Description of Artificial Sequence Synthetic polypeptide
QQEKNERDLLALDSWKNLWNWFDISNWLRYIQIFI

SEQ ID NO: 169 - protein, 35 aa, Artificial Sequence
  Description of Artificial Sequence Synthetic polypeptide
QQEKNEKDLLALDSWKNLWNWFSITNWLWYIKIFI

SEQ ID NO: 170 - protein, 35 aa, Artificial Sequence
  Description of Artificial Sequence Synthetic polypeptide
QQEKNERELLALDSWKNLWNWFDISNWLRYIQIFI

SEQ ID NO: 171 - protein, 35 aa, Artificial Sequence
  Description of Artificial Sequence Synthetic polypeptide
QQEKNEKDLLALDSWKNLWTWFDISNWLWYIRIFI

SEQ ID NO: 172 - protein, 35 aa, Artificial Sequence
  Description of Artificial Sequence Synthetic polypeptide
QQEENEKDLLALDSWKNLWTWFSISNWLWYIRIFI

SEQ ID NO: 173 - protein, 16 aa, Artificial Sequence
  Description of Artificial Sequence Synthetic peptide
SLWNWFNITNWLWYIK

SEQ ID NO: 174 - protein, 32 aa, Artificial Sequence
  Description of Artificial Sequence Synthetic polypeptide
QQEKNEQELLELDKWASLWNWFDITNWLWYIR

SEQ ID NO: 175 - protein, 7 aa, Homo sapiens
QQYGQST

SEQ ID NO: 176 - protein, 30 aa, Artificial Sequence
  Description of Artificial Sequence Synthetic polypeptide
KDLLALDSWKNLWNWFDITKWLWYIKIFII

SEQ ID NO: 177 - protein, 30 aa, Artificial Sequence
  Description of Artificial Sequence Synthetic polypeptide
KDLLALDKWNSLWSWFDITKWLWYIKIFIM

SEQ ID NO: 178 - protein, 30 aa, Artificial Sequence
  Description of Artificial Sequence Synthetic polypeptide
KDLLALDRWQNLWNWFDITNWLWYIKIFIM

SEQ ID NO: 179 - protein, 30 aa, Artificial Sequence
  Description of Artificial Sequence Synthetic polypeptide
QELLELDSWASLWNWFDISKWLWYIKIFIM

SEQ ID NO: 180 - protein, 30 aa, Artificial Sequence
  Description of Artificial Sequence Synthetic polypeptide
KDLLALDSWKNLWNWFDITNWLWYIKIFIM

SEQ ID NO: 181 - protein, 30 aa, Artificial Sequence
  Description of Artificial Sequence Synthetic polypeptide
QELLELDKWASLWNWFDITHWLWYIKIFIM

SEQ ID NO: 182 - protein, 30 aa, Artificial Sequence
  Description of Artificial Sequence Synthetic polypeptide
HELLELDKWASLWNWFDITRWLWYIKIFIM

SEQ ID NO: 183 - protein, 30 aa, Artificial Sequence
  Description of Artificial Sequence Synthetic polypeptide
EDLLALDKWDNLWNWFDISKWLWYIKIFIM

SEQ ID NO: 184 - protein, 30 aa, Artificial Sequence
  Description of Artificial Sequence Synthetic polypeptide
QDLLALDKWANLWNWFDISNWLWYIKIFIM

SEQ ID NO: 185 - protein, 30 aa, Artificial Sequence
  Description of Artificial Sequence Synthetic polypeptide
QDLLALDKWASLWNWFDISKWLWYIRIFIM

SEQ ID NO: 186 - protein, 30 aa, Artificial Sequence
  Description of Artificial Sequence Synthetic polypeptide
QDLLALDKWANLWNWFDVSKWLWYIKIFIM

SEQ ID NO: 187 - protein, 30 aa, Artificial Sequence
  Description of Artificial Sequence Synthetic polypeptide
KELLELDKWANLWSWFDISNWLWYIKIFII

SEQ ID NO: 188 - protein, 30 aa, Artificial Sequence
  Description of Artificial Sequence Synthetic polypeptide
QELLALDKWANLWNWFNITEWLWYIKIFIM

SEQ ID NO: 189 - protein, 30 aa, Artificial Sequence
  Description of Artificial Sequence Synthetic polypeptide
KDLLALDSWESLWSWFNITNWLWYIRIFIM

SEQ ID NO: 190 - protein, 30 aa, Artificial Sequence
  Description of Artificial Sequence Synthetic polypeptide
KDLLALDSWNNLWNWFNITNWLWYIKIFIM

SEQ ID NO: 191 - protein, 30 aa, Artificial Sequence
  Description of Artificial Sequence Synthetic polypeptide
LELLELDKWGSLWNWFSISNWLWYIRIFII

SEQ ID NO: 192 - protein, 30 aa, Artificial Sequence
  Description of Artificial Sequence Synthetic polypeptide
QDLLALDKWESLWNWFSITKWLWYIKIFIM

SEQ ID NO: 193 - protein, 30 aa, Artificial Sequence
  Description of Artificial Sequence Synthetic polypeptide
QELLALDKWASLWSWFSITHWLWYIKMFIM

SEQ ID NO: 194 - DNA, 369 bp, Artificial Sequence
  Description of Artificial Sequence Synthetic polynucleotide
caggtgcagctggtgcagtctggggcggaagtgaagaagcctgggtcctcggtgaagctctcctgtaaggcttctggaggcaccttcggcagctattctgtcacctgggtgcgccaggcccctggacaaacgtttgagtgggtgggcaggatcgtcccttgggttggtgttccgaactacgcaccgaagttccagggcagagtcaccattaccgcggacaaatcgagcacagtctacatggaattgaccagtctgagatttgaggacacggccgtctattactgtgcgacagcctatgaggcgagtgggttgtcatactactactacatggacgactggggcaaagggaccacggtcaccgtctcctca

SEQ ID NO: 195 - DNA, 330 bp, Artificial Sequence; identical to SEQ ID NO: 4
  Description of Artificial Sequence Synthetic polynucleotide

SEQ ID NO: 196 - DNA, 372 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagcctatgaggcgagtgggttgtcctactactactacatggacgtctggggcaaagggaccacggtcaccgtctcctca

SEQ ID NO: 197 - DNA, 330 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctgggatgtacacttttggccaggggaccaagctggagatcaaa

SEQ ID NO: 198 - DNA, 25 bp, Artificial Sequence
  Description of Artificial Sequence Synthetic primer
ctgggttccaggttccactggtgac

SEQ ID NO: 199 - DNA, 18 bp, Artificial Sequence
  Description of Artificial Sequence Synthetic primer
cacctctgggggcacagc

SEQ ID NO: 200 - DNA, 24 bp, Artificial Sequence
  Description of Artificial Sequence Synthetic primer
cgaactgtggctgcaccatctgtc

SEQ ID NO: 201 - DNA, 21 bp, Artificial Sequence
  Description of Artificial Sequence Synthetic primer
gctgccccctcggtcactctg